(19) United States
(12) Patent Application Publication
(10) Pub. No.: US 2024/0209334 A1
(43) Pub. Date: Jun. 27, 2024

(54) SMALL-MOLECULE-ACTIVATED GLYCAN MODIFYING ENZYMES AND USES THEREOF

(71) Applicant: President and Fellows of Harvard College, Cambridge, MA (US)

(72) Inventors: Christina M. Woo, Cambridge, MA (US); Yun Ge, Cambridge, MA (US)

(73) Assignee: President and Fellows of Harvard College, Cambridge, MA (US)

(21) Appl. No.: 18/394,724

(22) Filed: Dec. 22, 2023

Related U.S. Application Data
(60) Provisional application No. 63/478,493, filed on Jan. 4, 2023.
(60) Provisional application No. 63/477,133, filed on Dec. 23, 2022.

(51) Int. Cl. C12N 9/24; A61K 38/00; C12P 21/00; G01N 33/50; G01N 33/68

(52) U.S. Cl. CPC C12N 9/2402; C12P 21/005; G01N 33/502; G01N 33/68; A61K 38/00; C07K 2319/09; C07K 2319/095; C12Y 302/01169; G01N 2333/924

(57) ABSTRACT

The present disclosure provides glycosyl hydrolases (e.g., OGA) comprising a ligand-dependent intein. The present disclosure also provides pharmaceutical compositions comprising the glycosyl hydrolases disclosed herein, as well as polynucleotides, vectors, cells, and kits. Methods of using the disclosed intein-containing glycosyl hydrolases are also provided herein, including methods of deglycosylating a target protein. Methods of treating a glycosylation-associated disease in a subject, as well as methods of sensitizing a cell to a desirable therapeutic outcome, are also provided herein.

### RELATED APPLICATIONS

This application claims priority under 35 U.S.C. § 119(c) to U.S. Provisional Application, U.S. Ser. No. 63/477,133, filed Dec. 23, 2022, and U.S. Provisional Application, U.S. Ser. No. 63/478,493, filed Jan. 4, 2023, each of which is incorporated herein by reference.

### GOVERNMENT SUPPORT

This invention was made with government support under CA242098 awarded by National Institutes of Health (NIH). The government has certain rights in this invention.

### REFERENCE TO AN ELECTRONIC SEQUENCE LISTING

The contents of the electronic sequence listing (H082470418US02-SEQ-AZW.xml; Size: 124,0298 bytes; and Date of Creation: Dec. 14, 2023) is herein incorporated by reference in its entirety.

### BACKGROUND OF THE INVENTION

O-GlcNAc is a ubiquitous monosaccharide post-translational modification found on nucleocytoplasmic proteins across many species. The O-GlcNAc modification is orchestrated by a single pair of enzymes: O-GlcNAc transferase (OGT) for installation, and O-GlcNAcase (OGA) for removal. These enzymes dynamically regulate O-GlcNAc and hence many fundamental cellular processes in a spatiotemporal manner, responding to fluctuating nutrient levels, stresses, and signaling stimuli (1, 2). Maintenance of O-GlcNAc homeostasis is crucial for regular cellular activities and is achieved through multiple mechanisms, including translational (3, 4) and transcriptional regulation (5, 6). Not surprisingly, abnormal O-GlcNAcylation is implicated in many diseases (7-9). For example, many of these O-GlcNAcylated proteins are known to be associated with oncogenesis. A persistent hyper-O-GlcNAcylation state is commonly observed in various cancers (10), such as breast, prostate, and lung cancer, implying a potential role in tumor progression and metastasis.

A method to control O-GlcNAcylation with spatial and temporal resolution would enable connection of these dynamic features of O-GlcNAc to biological functions. Global changes to O-GlcNAc through chemical inhibitors or genetic manipulation of OGT (3) and OGA (11), target protein selective methods (12, 13), and site-specific point mutagenesis approaches (14) have limited spatial and temporal resolution. Growing efforts towards spatiotemporal control of enzymatic function (15) have recently provided a new chemical biology approach to enhance O-GlcNAc in the form of a photo-activatable OGT (16). Generation of this photo-activatable OGT was achieved through genetic code expansion to afford an approach to spatiotemporally increase protein O-GlcNAcylation (16) (FIG. 1A). In contrast, methods to reduce O-GlcNAc with spatiotemporal control have primarily focused on chemically-triggered strategies to reduce OGT, including a 4-HT-triggered conditional OGT knockout MEF cell line (17) and a rapamycin-induced OGT degradation strategy using the dTAG technique (18), which provides some temporal resolution but requires a long incubation time to take effect (FIG. 1A). Because O-Glc-NAc regulation by OGT and OGA may not be directly equivalent (19), a method to crase O-GlcNAc through spatiotemporal control of OGA would provide a complementary strategy for methods that manipulate OGT.

### SUMMARY OF THE INVENTION

The present disclosure describes the design of an approach facilitating controllable activation of OGA to manipulate O-GlcNAc in a dose-dependent and time-resolved manner (FIG. 1B). Inteins have evolved to undergo splicing upon treatment with 4-hydroxytamoxifen (4-HT) (20, 21), have widespread applications in expressed protein ligation (22, 23), and have been used for the small molecule-triggered activation of Cas9 for increased gene editing specificity (24). A similar approach was implemented to produce an engineered OGA with the evolved intein inserted at an optimized site. Upon treatment with 4-HT, the intein self-splices from the OGA-intein fusion, thus enabling refolding of OGA and thereby restoring its catalytic activity for removal of O-GlcNAc. This strategy enabled OGA activation and corresponding reduction of O-GlcNAc with dose and time controls. In addition, by targeting OGA variants to different subcellular localizations (e.g., through the use of nuclear localization sequences (NLS) and nuclear export sequences (NES)), O-GlcNAc removal in specific subcellular compartments was successfully performed, demonstrating additional control over the spatial dimension. 4-HT is the active metabolite of tamoxifen and is a selective estrogen receptor (ER) modulator that is widely used in the therapeutic and chemo-preventive treatment of breast cancer (25) but can induce drug resistance after extended treatment (26). The dual functionalities of 4-HT on antagonizing ER and activating OGA-intein for inhibition of MCF-7 cell growth at a lower dose were also leveraged. Removal of O-GlcNAc may also be used to sensitize cells to therapy, for example cancer therapy.

Thus, in one aspect, the present disclosure provides glycosyl hydrolases comprising an intein (e.g., an intein is inserted at a position within the glycosyl hydrolase). In some embodiments, the activity of the glycosyl hydrolase is disrupted by the intein and restored upon excision of the intein. In some embodiments, the glycosyl hydrolase is an O-GlcNAcase (OGA), e.g., a split OGA or a mini OGA. In certain embodiments, the OGA comprises the structure NH₂-[catalytic domain]-[first portion of stalk domain]-[linker]-[second portion of stalk domain]-COOH. The linker may comprise one or more repeats of the sequence GS, for example, the sequence GSGSGSGSGSGSGSG (SEQ ID NO: 1). In some embodiments, the intein can be inserted in the catalytic domain. In other embodiments, the intein can be inserted in the stalk domain, for example in the first portion or the second portion of the stalk domain. In certain embodiments, the glycosyl hydrolase comprises the amino acid sequence of SEQ ID NO: 107, or an amino acid sequence at least 80%, at least 85%, at least 90%, at least 95%, or at least 99% identical to the amino acid sequence of SEQ ID NO: 107. In certain embodiments, the glycosyl hydrolase comprising the intein comprises the amino acid sequence of SEQ ID NO: 108, or an amino acid sequence at least 80%, at least 85%, at least 90%, at least 95%, or at least 99% identical to the amino acid sequence of SEQ ID NO: 108. In certain embodiments, the glycosyl hydrolase comprising the intein with D174N comprises the amino acid sequence of SEQ ID NO: 109, or an amino acid sequence at least 80%, at least 85%, at least 90%, at least 95%, or at least 99% identical to the amino acid sequence of SEQ ID NO: 109. In certain embodiments, the glycosyl hydrolase comprising the intein and an NLS comprises the amino acid sequence of SEQ ID NO: 110, or an amino acid sequence at least 80%, at least 85%, at least 90%, at least 95%, or at least 99% identical to the amino acid sequence of SEQ ID NO: 110. In certain embodiments, the glycosyl hydrolase comprising the intein and an NES comprises the amino acid sequence of SEQ ID NO: 111, or an amino acid sequence at least 80%, at least 85%, at least 90%, at least 95%, or at least 99% identical to the amino acid sequence of SEQ ID NO: 111. This sequences are exemplary, and are not meant to be limiting as to the linker, the glycosyl hydrolase, the NLS, or the NES.

Any intein described herein or known in the art may be used in the glycosyl hydrolases provided herein. In some embodiments, the intein is a ligand-dependent intein that, for example, is excised from the glycosyl hydrolase upon being contacted with a ligand. In some embodiments, the ligand is a small molecule, a peptide, a protein, an amino acid, a polynucleotide, or a nucleic acid. In certain embodiments, the ligand is a small molecule (e.g., a cell permeable and nontoxic small molecule). In certain embodiments, the ligand is 4-hydroxytamoxifen (4HT). The intein may be inserted at any position within the glycosyl hydrolase. In some embodiments, the intein is inserted at or replaces a cysteine within the glycosyl hydrolase. In some embodiments, the intein is inserted at or replaces C62, C166, C181, C220, C316, C596, C631, or C663 in SEQ ID NO: 107. In certain embodiments, the intein is inserted at or replaces C181 in SEQ ID NO: 107. In some embodiments, the intein can be inserted in the catalytic domain. In other embodiments, the intein can be inserted in the stalk domain, for example in the first portion or the second portion of the stalk domain. In some embodiments, the intein comprises the amino acid sequence of any of SEQ ID NOs: 2-9, or an amino acid sequence at least 80%, at least 85%, at least 90%, at least 95%, or at least 99% identical to the amino acid sequence of any of SEQ ID NOs: 2-9.

In some aspects, the glycosyl hydrolases provided herein may be further modified in order to target them to particular subcellular locations (e.g., nucleus). In some aspects, the glycosyl hydrolases provided herein may be further modified in order to target them to particular proteins of interest. In some embodiments, the glycosyl hydrolases are fused to a nuclear localization sequence (NLS). The NLS may facilitate targeting of the glycosyl hydrolase to the nucleus of a cell and/or selective spatial deglycosylation in the nucleus. In some embodiments, the glycosyl hydrolase is fused to a nuclear export sequence (NES). The NES may facilitate targeting of the glycosyl hydrolase to the cytoplasm of a cell and/or selective spatial deglycosylation in the cytoplasm of a cell. In some embodiments, the glycosyl hydrolases provided herein are fused to a targeting molecule. In certain embodiments, the targeting molecule facilitates targeting of the glycosyl hydrolase to a particular protein target, such as for example a cell surface protein. In certain embodiments, the targeting molecule facilities targeting of the glycosyl hydrolase to protein target or tag, such as for example Green Fluorescent Protein, EPEA, or UBC6e. In certain embodiments, the targeting molecule facilitates targeting of the glycosyl hydrolase to a particular cell type. In some embodiments, the targeting molecule is an antibody, or a fragment thereof (e.g., a nanobody).

In another aspect, the present disclosure provides pharmaceutical compositions comprising any of the glycosyl hydrolases disclosed herein and a pharmaceutically acceptable excipient.

In another aspect, the present disclosure provides polynucleotides encoding any of the glycosyl hydrolases disclosed herein.

In another aspect, the present disclosure provides vectors comprising any of the polynucleotides encoding any of the glycosyl hydrolases disclosed herein.

In another aspect, the present disclosure provides cells comprising any of the glycosyl hydrolases, polynucleotides, or vectors disclosed herein.

In another aspect, the present disclosure provides kits comprising any of the glycosyl hydrolases, polynucleotides, or vectors disclosed herein.

In another aspect, the present disclosure provides methods of deglycosylating a target protein. In some embodiments, the methods comprise: (i) contacting a target protein containing a sugar moiety with any of the glycosyl hydrolases provided herein, and (ii) contacting the glycosyl hydrolase with a ligand, thereby excising the intein from the glycosyl hydrolase and restoring its activity. In some embodiments, the sugar moiety is removed from the target protein upon restoration of the activity of the glycosyl hydrolase. In certain embodiments, the sugar moiety is an O-linked N-acetyl glucosamine. In certain embodiments, the O-linked N-acetyl glucosamine is removed from a serine or threonine residue of the target protein. In some embodiments, the method is performed in a cell. In certain embodiments, the cell is in a subject (e.g., a human).

In another aspect, the present disclosure provides methods of studying the effects of glycosylation on protein function in one or more cells using any of the glycosyl hydrolases provided herein.

In another aspect, the present disclosure provides methods of treating a glycosylation-associated disease in a subject (e.g., a neurodegenerative disease (Parkinson's disease, Huntington's disease, Alzheimer's disease, dementia, multiple system atrophy), cancer, or diabetes). In some embodiments, the methods comprise: (i) administering to the subject a therapeutically effective amount of any of the glycosyl hydrolases provided herein, and (ii) contacting the glycosyl hydrolase with a ligand, thereby excising the intein from the glycosyl hydrolase and restoring its activity.

In some aspects, the methods provided herein are used for reducing drug resistance in a cell by modulating the glycosylation state of one or more proteins in the cell using any of the glycosyl hydrolases provided herein. In some aspects, the methods provided herein are used for sensitizing a cell to a desirable therapeutic outcome by modulating the glycosylation state of one or more proteins in the cell using any of the glycosyl hydrolases provided herein. In some embodiments, the cell is a cancer cell.

Other advantages, features, and uses of the invention will be apparent from the detailed description of certain exemplary, non-limiting embodiments, the drawings, the non-limiting working examples, and the claims.

### BRIEF DESCRIPTION OF THE DRAWINGS

FIGS. 1A-1B depict the design of 4-hydroxytamoxifen (4-HT)-induced engineered OGA activation for spatial and temporal downregulation of O-GlcNAc level in living cells. FIG. 1A shows previous approaches for spatiotemporal control of O-GlcNAc, primarily through regulation of OGT. FIG. 1B shows that 4-HT can bind the engineered intein and induce its release from OGA to activate OGA's activity for de-O-GlcNAcylation with dose and temporal controls. Localization of OGA to different subcellular loci will acquire spatial resolution.

FIGS. 2A-2C show design and optimization of intein insertion sites to miniOGA and evaluation of 4-HT-dependent activation of the OGA-intein fusion. FIG. 2A depicts a schematic of miniOGA structure and insertion sites screened in this study. Sequence shown corresponds to SEQ ID NO: 1. FIGS. 2B-2C show that catalytic activity of the indicated OGA-intein variants was evaluated on the glycoprotein Nup62 in the absence or presence of 4-HT. Nup62 bearing the EPEA tag was co-expressed with the indicated construct, enriched by anti-EPEA beads, and analyzed by immunoblotting to reveal the protein level and O-GlcNAc modification level. WCL, whole-cell lysate.

FIGS. 3A-3C show dose- and time-dependent activation of OGA-intein (C181) by 4-HT in HEK293T cells. FIG. 3A shows the analysis of OGA-intein(C181) activation and deglycosylation when applying a series of 4-HT concentrations on co-expressed Nup62. FIG. 3B shows the measurement of hexoaminidase activity on cells after 4-HT treatment and lysis by an in vitro OGA activity assay. Data are shown as the mean ±s.d. of n=3 independent experiments. FIG. 3C shows immunoblotting analysis of time-dependent activation of OGA-intein(C181) at different 4-HT treatment time points on co-expressed Nup62 in HEK293T cells. Nup62 was enriched by the indicated tag for evaluation of O-GlcNAc levels. WCL, whole-cell lysate.

FIGS. 4A-4F show subcellular specific de-O-GlcNAcylation mediated by activation of OGA-intein(C181) at a desired localization in HEK293T cells. FIG. 4A shows subcellular distributions of the indicated OGA-intein(C181) variant before and after adding 4-HT for 24 hours. OGA-intein variants with a Myc tag and nuclei are shown. Co-localization between OGA-intein variants and the nucleus is assessed by Pearson's correlation coefficient. Unpaired two-tailed Student's t-tests were used for statistical analysis, **** P<0.0001. Images are representative of at least three randomly selected frames. FIG. 4B shows that OGA-intein(C181)-NES (C181-NES), and not OGA-intein(C181)-NLS (C181-NLS), removes O-GlcNAc from cytosolic Nup62. FIG. 4C shows that OGA-intein(C181)-NLS, and not OGA-intein(C181)-NES, removes O-GlcNAc from nuclear Nup62-NLS. D174N, the catalytic inactive mutant of min-iOGA. WCL, whole-cell lysate. FIG. 4D is a volcano plot of identified O-GlcNAcylated proteins in HEK293T cells expressing C181-NLS with and without 4-HT treatment after 24 hours. P=0.05 and +0.5-log 2 (fold change) are denoted by gray dashed lines as the significant threshold. Each point represents an individual protein from three independent biological replicates in each condition. Nuclear and cytosolic proteins are indicated by white and black points, respectively. FIG. 4E provides the top 5 cellular compartment terms overrepresented in significantly decreased O-GlcNAcylated proteins identified in FIG. 4D using Gene Ontology analysis.

FIGS. 5A-5B depict suppression of MCF-7 cell viability with OGA-intein activation by 4-HT in a dual-functional way. FIG. 5A provides a depiction of the working model of 4-HT-mediated MCF-7 cell death. FIG. 5B shows the relative cell viabilities measured by CCK-8 assay with the treatment of increasing doses of 4-HT on indicated MCF-7 cell lines after 48 hours. Data are shown as the mean ±s.d. of at least n=3 independent experiments. Unpaired two-tailed Student's t-tests were used for statistical analysis of MCF7-C181 and MCF7-C181_D174N, **** P<0.0001.

FIG. 6 depicts a schematic showing that O-GlcNAcase is engineered for 4-hydroxytamoxifen-triggered activation in a dose- and time-dependent manner, serving as a novel tool for spatiotemporal downregulation of O-GlcNAcylation in live cells.

FIG. 7 shows immunoblotting analysis of a dose-course experiment of 4-HT-triggered OGA-intein(C181) activation in HEK293T cells. HEK293T cells co-expressing Nup62 and the indicated OGA variants were treated with 4-HT at varied concentrations for 24 hours. Nup62 was enriched and blotted with the RL2 antibody to reveal O-GlcNAc modification level. WCL, whole-cell lysate.

FIG. 8 shows immunoblotting analysis results of the whole cell lysates from the experiment shown on FIG. 3B. Immunoblotting analysis of the increasing accumulation of spliced product and global reduction of O-GlcNAc level resulting from the elongation of incubation time of 4-HT using the indicated antibodies. Along with the increased incubation time, OGT's protein level shows an obvious increase while the decrease of OGA's protein level is subtle. The asterisk * indicated the endogenous OGA and ** indicated the OGA-intein(C181) before splicing.

FIGS. 9A-9B show immunoblotting and RT-PCR analyses of cell samples with a time-dependent activation of OGA-intein(C181) by 4-HT from the experiments shown in FIG. 3C. FIG. 9A shows immunoblotting analysis of global O-GlcNAc level on whole cell lysates from FIG. 3C. FIG. 9B shows the relative mRNA level of endogenous OGT and OGA in cells expressing the indicated constructs with the treatment of 4-HT at different time points by quantitative RT-PCR. Data are shown as the mean ±s.d. of n=3 independent experiments. C181 represents OGA-intein(C181).

FIGS. 10A-10D shows confocal imaging of subcellular localizations of the indicated OGA-intein(C181) variants used in this study in HEK293T cells. FIG. 10A shows that the full-length OGA and miniOGA were distributed in both cytoplasm and nucleus when overexpressed in cells. FIGS. 10B-10D show single-channel and merged images of samples from the experiments shown in FIG. 4A. OGA-intein variants bearing a Myc tag were labeled by Myc-Tag (9B11) mouse primary antibody and Alexa Fluor™ 568 secondary antibody sequentially. Nucleus was stained with NucBlue™ Fixed Cell Stain ReadyProbes™ reagent. Scale bar: 10 μm. Right: merged channel. Proteins expressed with or without 4-HT in each sample were annotated on the left side.

FIGS. 11A-11C show confocal imaging of subcellular localizations of indicated Nup62 variants co-expressed with OGA-intein(C181) variants used in this study in HEK293T cells. FIG. 11A shows that OGA-intein(C181)-NLS colocalized with nuclear Nup62-NLS-His after adding 4-HT for activation. FIG. 11B shows the cytoplasmic OGA-intein(C181)-NES colocalized with Nup62-His before and after the addition of 4-HT for activation. The intensity profiles of OGA-intein variants, Nup62 and DAPI along the white line in the selected cell are plotted in the right panels. FIG. 11C shows the co-localization between OGA-intein variants and Nucleus is assessed by Pearson's correlation coefficient. The number of cells analyzed in each group was shown on the top. OGA-intein variants bearing a Myc tag were labeled by Myc-Tag (9B11) mouse primary antibody and Alexa Fluor™ 568 secondary antibody sequentially. Nup62 variants with a His tag were labeled by His-Tag (12698) rabbit primary antibody and Alexa Fluor™ 488 Nucleus was stained with NucBlue™ Fixed Cell Stain ReadyProbes™ reagent. Scale bar: 10 μm. Right: merged channel. Proteins expressed with or without 4-HT in each sample were annotated on the left side. Unpaired two-tailed Student's t-tests were used for statistical analysis, **** P<0.0001. Images are representative of at least three randomly selected frames.

FIG. 12A-12C show mass spectrometry analyses of 4-HT-induced activation of OGA-intein(C181)-NLS for specific deglycosylation in the nucleus. FIG. 12A is a volcano plot illustrating the comparison of enriched O-GlcNAcylated proteins in HEK293T cells with and without the treatment of 25 μM OGT inhibitor OSMI-4b for 10 hours. FIG. 12B is a volcano plot illustrating the comparison of identified O-GlcNAcylated proteins in HEK293T cells expressing C181-NLS with and without the treatment of 1 μM 4-HT for 2 hours. P=0.05 and +0.5-log 2 (fold change) are denoted by gray dashed lines as the significance threshold. Each point represents an individual identified protein from three independent biological replicates. Cytoplasmic proteins (white) and nuclear proteins (black) were highlighted according to the annotation from the UniProt Database. FIG. 12C shows the comparison of accumulated percentage of nuclear O-GlcNAcylated proteins with reduced fold change between results shown on FIG. 4D (white) and FIG. 12A (gray). More nuclear proteins were enriched in the proteome fraction with reduced O-GlcNAcylation after the indicated treatment.

FIG. 13 shows a significant correlation between high mRNA expression of OGA and increased survival of patients with ER positive breast cancer. The analysis used ER positive breast cancer, RFS survival type, and auto select best cutoff. The plot was generated by an online Kaplan-Meier Plotter tool (48) (kmplot.com/analysis/).

FIGS. 14A-14B show generation of MCF-7 cell lines stably expressing OGA-intein(C181) or its inactive variant. FIG. 14A shows that MCF-7 cells infected with lentivirus carrying a GFP selection marker were sorted by a flow cytometer. FIG. 14B show that both MCF-7 stable cell lines are able to express OGA-intein(C181) variants at comparable levels, which can be induced to splice upon adding 4-HT. MCF-7 cells expressing OGA-intein(C181), instead of cells expressing the inactive form (C181_D174N), decreased global O-GlcNAc level after adding 4-HT.

FIGS. 15A-15B show cell death analyses of MCF-7 cell lines stably expressing OGA-intein(C181) (MCF7-C181) or its inactive variant (MCF7-C181_D174N) under the treatment of 10 μM 4-HT for 24 hours. FIG. 15A shows MCF-7 cell lines cultured with 1% FBS, in the presence or absence of 10 μM 4-HT. After 24 hours, cells were collected and stained with Annexin V-mCherry/DAPI for apoptosis analysis. FIG. 15B provides a histogram showing percentages of early apoptotic cells (Q3) and late apoptotic/necrotic cells (Q2).

FIG. 16 shows 4-HT-induced time-dependent activation of OGA-intein(C181) stably expressed in MCF-7 cells. MCF-7 cells stably expressing OGA-intein(C181) wereincubated with 1 μM 4-HT for the indicated time. The increasing active splicing product and the decreasing global O-GlcNAc level were analyzed by immunoblots against Mycand O-GlcNAc (RL2), respectively. The result is representative of two biological replicates.

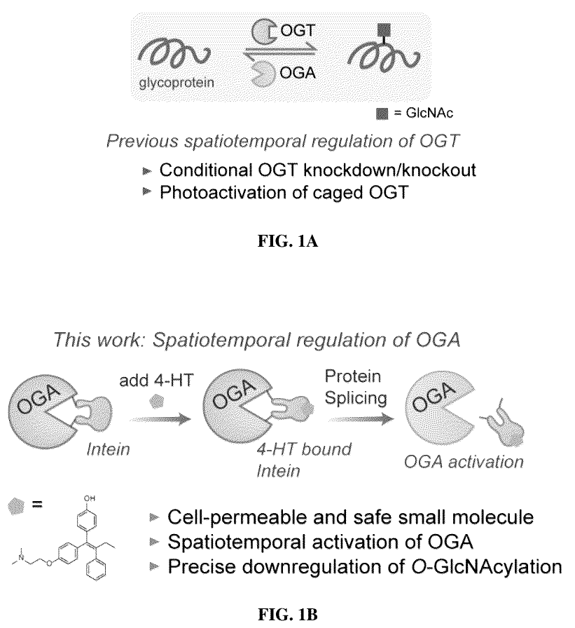

### DEFINITIONS

The term “glycosyl hydrolase” (also referred to as a “glycoside hydrolase” or “glycosidase”), as used herein, refers to a class of enzymes capable of catalyzing the hydrolysis of glycosidic bonds in complex sugars (i.e., polysaccharides comprising more than one carbohydrate monomer). Some glycosyl hydrolases, for example, O-GlcNAcase (OGA), catalyze the removal of sugar moieties from post-translationally modified proteins. Glycosyl hydrolases can be from any species, can include any variant, and can be in any form. Exemplary glycosyl hydrolases include, but are not limited to, α-amylase, β-amylase, glucan 1,4-α-glucosidase, cellulase, endo-1,3(4)-β-glucanase, inulinase, endo-1,4-β-xylanase, oligo-1,6-glucosidase, dextranase, chitinase, polygalacturonase, lysozyme, exo-α-sialidase, α-glucosidase, β-glucosidase, α-galactosidase, β-galactosidase, α-mannosidase, β-mannosidase, β-fructofuranosidase, α,α-trehalase, β-glucuronidase, endo-1,3-β-xylanase, amylo-1,6-glucosidase, hyaluronoglucosaminidase, hyaluronoglucuronidase, xylan 1,4-β-xylosidase, β-D-fucosidase, glucan endo-1,3-β-D-glucosidase, α-L-rhamnosidase, pullulanase, GDP-glucosidase, β-L-rhamnosidase, fucoidanase, glucosylceramidase, galactosylceramidase, galactosylgalactosylglucosylceramidase, sucrose α-glucosidase, α-N-acetylgalactosaminidase, α-N-acetylglucosaminidase, α-L-fucosidase, β-L-N-acetylhexosaminidase, β-N-acetylgalactosaminidase, cyclomaltodextrinase, non-reducing end α-L-arabinofuranosidase, glucuronosyl-disulfoglucosamine glucuronidase, isopullulanase, glucan 1,3-β-glucosidase, glucan endo-1,3-α-glucosidase, glucan 1,4-α-maltotetraohydrolase, mycodextranase, glycosylceramidase, 1,2-α-L-fucosidase, 2,6-β-fructan 6-levanbiohydrolase, levanase, quercitrinase, galacturan 1,4-α-galacturonidase, isoamylase, glucan 1,6-α-glucosidase, glucan endo-1,2-β-glucosidase, xylan 1,3-β-xylosidase, licheninase, glucan 1,4-β-glucosidase, glucan endo-1,6-β-glucosidase, L-iduronidase, mannan 1,2-(1,3)-α-mannosidase, mannan endo-1,4-β-mannosidase, fructan β-fructosidase, β-agarase, exo-poly-α-galacturonosidase, k-carrageenase, glucan 1,3-α-glucosidase, 6-phospho-β-galactosidase, 6-phospho-β-glucosidase, capsular-polysaccharide endo-1,3-α-galactosidase, non-reducing end β-L-arabinopyranosidase, arabinogalactan endo-β-1,4-galactanase, cellulose 1,4-β-cellobiosidase (non-reducing end), peptidoglycan β-N-acetylmuramidase, α,α-phosphotrehalase, glucan 1,6-α-isomaltosidase, dextran 1,6-α-isomaltotriosidase, mannosyl-glycoprotein endo-β-N-acetylglucosaminidase, endo-α-N-acetylgalactosaminidase, glucan 1,4-α-maltohexaosidase, arabinan endo-1,5-α-L-arabinanase, mannan 1,4-mannobiosidase, mannan endo-1,6-α-mannosidase, blood-group-substance endo-1,4-β-galactosidase, keratan-sulfate endo-1,4-β-galactosidase, steryl-β-glucosidase, strictosidine β-glucosidase, mannosyl-oligosaccharide glucosidase, protein-glucosylgalactosylhydroxylysine glucosidase, lactase, endogalactosaminidase, 1,3-α-L-fucosidase, 2-deoxyglucosidase, mannosyl-oligosaccharide 1,2-α-mannosidase, mannosyl-oligosaccharide 1,3-1,6-α-mannosidase, branched-dextran exo-1,2-α-glucosidase, glucan 1,4-α-maltotriohydrolase, amygdalin β-glucosidase, prunasin β-glucosidase, vicianin β-glucosidase, oligoxyloglucan β-glycosidase, polymannuronate hydrolase, maltose-6′-phosphate glucosidase, endoglycosylceramidase, 3-deoxy-2-octulosonidase, raucaffricine β-glucosidase, coniferin β-glucosidase, 1,6-α-L-fucosidase, glycyrrhizinate β-glucuronidase, endo-α-sialidase, glycoprotein endo-α-1,2-mannosidase, xylan α-1,2-glucuronosidase, chitosanase, glucan 1,4-α-maltohydrolase, difructose-anhydride synthase, neopullulanase, glucuronoarabinoxylan endo-1,4-β-xylanase, mannan exo-1,2-1,6-α-mannosidase, α-glucuronidase, lacto-N-biosidase, 4-α-D-{(1→4)-α-D-glucano} trehalose trehalohydrolase, limit dextrinase, poly(ADP-ribose)glycohydrolase, 3-deoxyoctulosonase, galactan 1,3-β-galactosidase, β-galactofuranosidase, thioglucosidase, β-primeverosidase, oligoxyloglucan reducing-end-specific cellobiohydrolase, xyloglucan-specific endo-β-1,4-glucanase, mannosylglycoprotein endo-β-mannosidase, fructan β-(2,1)-fructosidase, fructan β-(2,6)-fructosidase, xyloglucan-specific exo-β-1,4-glucanase, oligosaccharide reducing-end xylanase, 1-carrageenase, α-agarase, α-neoagaro-oligosaccharide hydrolase, β-apiosyl-β-glucosidase, λ-carrageenase, 1,6-α-D-mannosidase, galactan endo-1,6-β-galactosidase, exo-1,4-β-D-glucosaminidase, heparanase, baicalin-β-D-glucuronidase, hesperidin 6-O-α-L-rhamnosyl-β-D-glucosidase, protein O-GlcNAcase, mannosylglycerate hydrolase, rhamnogalacturonan hydrolase, unsaturated rhamnogalacturonyl hydrolase, rhamnogalacturonan galacturonohydrolase, rhamnogalacturonan rhamnohydrolase, β-D-glucopyranosyl abscisate β-glucosidase, cellulose 1,4-β-cellobiosidase (reducing end), α-D-xyloside xylohydrolase, β-porphyranase, gellan tetrasaccharide unsaturated glucuronyl hydrolase, unsaturated chondroitin disaccharide hydrolase, galactan endo-β-1,3-galactanase, 4-hydroxy-7-methoxy-3-oxo-3,4-dihydro-2H-1,4-benzoxazin-2-yl glucoside β-D-glucosidase, UDP-N-acetylglucosamine 2-epimerase (hydrolysing), UDP-N,N′-diacetylbacillosamine 2-epimerase (hydrolysing), non-reducing end-L-arabinofuranosidase, protodioscin 26-O-β-D-glucosidase, (Ara-f)3-Hyp β-L-arabinobiosidase, avenacosidase, dioscin glycosidase (diosgenin-forming), dioscin glycosidase (3-O-β-D-Glc-diosgenin-forming), ginsenosidase type III, ginsenoside Rb1β-glucosidase, ginsenosidase type I, ginsenosidase type IV, 20-O-multi-glycoside ginsenosidase, limit dextrin α-1,6-maltotetraose-hydrolase, β-1,2-mannosidase, α-mannan endo-1,2-α-mannanase, sulfoquinovosidase, exo-chitinase (non-reducing end), exo-chitinase (reducing end), endo-chitodextinase, carboxymethylcellulase, 1,3-α-isomaltosidase, isomaltose glucohydrolase, oleuropein β-glucosidase, and mannosyl-oligosaccharide α-1,3-glucosidase. In some embodiments the glycosyl hydrolase is selected from the group consisting of purine nucleosidase, inosine nucleosidase, uridine nucleosidase, AMP nucleosidase, NAD⁺ glycohydrolase, ADP-ribosyl cyclase/cyclic ADP-ribose hydrolase, adenosine nucleosidase, ribosylpyrimidine nucleosidase, adenosylhomocysteine nucleosidase, pyrimidine-5′-nucleotide nucleosidase, β-aspartyl-N-acetylglucosaminidase, inosinate nucleosidase, 1-methyladenosine nucleosidase, NMN nucleosidase, DNA-deoxyinosine glycosylase, methylthioadenosine nucleosidase, deoxyribodipyrimidine endonucleosidase, ADP-ribosylarginine hydrolase, DNA-3-methyladenine glycosylase I, DNA-3-methyladenine glycosylase II, rRNA N-glycosylase, DNA-formamidopyrimidine glycosylase, ADP-ribosyl-[dinitrogen reductase] hydrolase, N-methyl nucleosidase, futalosine hydrolase, uracil-DNA glycosylase, double-stranded uracil-DNA glycosylase, thymine-DNA glycosylase, aminodeoxyfutalosine nucleosidase, and adenine glycosylase. In certain embodiments, the glycosyl hydrolase is O-GlcNAcase (OGA).

The term “split OGA,” as used herein, refers to a glycosyl hydrolase that has been split into two separate pieces. Split glycosyl hydrolases are described, for example, in PCT publication WO 2022/076329 and Ge, Y. et al., “Target protein deglycosylation in living cells by a nanobody-fused split O-GlcNAcase.” Nat. Chem. Biol. 2021, 17, 593, each of which is incorporated herein by reference. A split OGA may comprise a first piece comprising a catalytic domain and a second piece comprising a stalk domain. In some embodiments, the catalytic domain is a truncated catalytic domain. In some embodiments, the stalk domain is a truncated stalk domain. In certain embodiments, the catalytic domain is a truncated catalytic domain, and the stalk domain is a truncated stalk domain.

The term “mini OGA,” as used herein, refers to an OGA variant comprising a truncation of the C-terminal HAT domain and comprising the structure NH₂-[catalytic domain]-[first portion of stalk domain]-[linker]-[second portion of stalk domain]-COOH. The “HAT domain” refers to a histone acetyltransferase domain. Histone acetyltransferases are enzyme that transfer an acetyl group from acetyl-CoA to conserved lysine amino acid residues on histone proteins. OGA enzymes comprise a HAT domain and display histone acetyltransferase activity in vitro.

The term “intein,” as used herein, refers to an amino acid sequence that is capable of excising itself from a protein and rejoining the remaining protein segments (the exteins) via a peptide bond in a process termed protein splicing. Inteins are analogous to the introns found in mRNA. Many naturally occurring and engineered inteins and hybrid proteins comprising such inteins are known to those of skill in the art, and the mechanism of protein splicing has been the subject of extensive research. As a result, methods for the generation of hybrid proteins from naturally occurring and engineered inteins are well known to the skilled artisan. Sec Gross, Belfort, Derbyshire, Stoddard, and Wood (Eds.) Homing Endonucleases and Inteins Springer Verlag Heidelberg, 2005; ISBN 9783540251064; the contents of which are incorporated herein by reference for disclosure of inteins and methods of generating hybrid proteins comprising natural or engineered inteins. As will be apparent to those of skill in the art, an intein may catalyze protein splicing in a variety of extein contexts. Accordingly, an intein can be introduced into virtually any target protein sequence, including any glycosyl hydrolase (e.g., OGA), to create a desired hybrid protein.

The term “ligand-dependent intein,” as used herein, refers to an intein that comprises a ligand-binding domain. Typically, the ligand-binding domain is inserted into the amino acid sequence of the intein, resulting in the structure intein (N)-ligand-binding domain-intein (C). Typically, ligand-dependent inteins exhibit no or only minimal protein splicing activity in the absence of a cognate ligand, and a marked increase of protein splicing activity in the presence of the ligand. In some embodiments, the ligand-dependent intein does not exhibit observable splicing activity in the absence of its ligand but does exhibit splicing activity in the presence of the ligand. In some embodiments, the ligand-dependent intein exhibits an observable protein splicing activity in the absence of the ligand, and a protein splicing activity in the presence of an appropriate ligand that is at least 2 times, at least 5 times, at least 10 times, at least 50 times, at least 100 times, at least 150 times, at least 200 times, at least 250 times, at least 500 times, at least 1000 times, at least 1500 times, at least 2000 times, at least 2500 times, at least 5000 times, at least 10000 times, at least 20000 times, at least 25000 times, at least 50000 times, at least 100000 times, at least 500000 times, or at least 1000000 times greater than the activity observed in the absence of the ligand. In some embodiments, the increase in activity is dose dependent over at least 1 order of magnitude, at least 2 orders of magnitude, at least 3 orders of magnitude, at least 4 orders of magnitude, or at least 5 orders of magnitude, allowing for fine-tuning of intein activity by adjusting the concentration of the ligand. Suitable ligand-dependent inteins are known in the art and include those provided below and those described in published U.S. Patent Application Publication No. 2014/0065711 A1; Mootz et al., “Protein splicing triggered by a small molecule.” J. Am. Chem. Soc. 2002, 124, 9044-9045; Mootz et al., “Conditional protein splicing: a new tool to control protein structure and function in vitro and in vivo.” J. Am. Chem. Soc. 2003, 125, 10561-10569; Buskirk et al., Proc. Natl. Acad. Sci. USA. 2004, 101, 10505-10510); Skretas & Wood, “Regulation of protein activity with small-molecule-controlled inteins.” Protein Sci. 2005, 14, 523-532; Schwartz et al., “Post-translational enzyme activation in an animal via optimized conditional protein splicing.” Nat. Chem. Biol. 2007, 3, 50-54; and Peck et al., Chem. Biol. 2011, 18(5), 619-630; the contents of each of which are incorporated herein by reference.

2-4 intein:
(SEQ ID NO: 2)
CLAEGTRIFDPVTGTTHRIEDVVDGRKPIHVVAAAKDGTLLARPV
VSWFDQGTRDVIGLRIAGGAIVWATPDHKVLTEYGWRAAGELRKG
DRVAGPGGSGNSLALSLTADQMVSALLDAEPPILYSEYDPTSPFS
EASMMGLLTNLADRELVHMINWAKRVPGFVDLTLHDQAHLLECAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEELRYSVIREVLPTRRARTFDLEVEELHTLVA
EGVVVHNC
3-2 intein:
(SEQ ID NO: 3)
CLAEGTRIFDPVTGTTHRIEDVVDGRKPIHVVAVAKDGTLLARPV
VSWFDQGTRDVIGLRIAGGAIVWATPDHKVLTEYGWRAAGELRKG
DRVAGPGGSGNSLALSLTADQMVSALLDAEPPILYSEYDPTSPFS
EASMMGLLTNLADRELVHMINWAKRVPGFVDLTLHDQAHLLECAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYTNVVPLYDLLLEMLDAHRLHAGGSGASRVQAFA
DALDDKFLHDMLAEELRYSVIREVLPTRRARTFDLEVEELHTLVA
EGVVVHNC
30R3-1 intein:
(SEQ ID NO: 4)
CLAEGTRIFDPVTGTTHRIEDVVDGRKPIHVVAAAKDGTLLARPV
VSWFDQGTRDVIGLRIAGGATVWATPDHKVLTEYGWRAAGELRKG
DRVAGPGGSGNSLALSLTADQMVSALLDAEPPIPYSEYDPTSPFS
EASMMGLLTNLADRELVHMINWAKRVPGFVDLTLHDQAHLLECA
WLEILMIGLVWRSMEHPGKLLFAPNLLLDRNQGKCVEGMVEIFDM
LLATSSRFRMMNLQGEEFVCLKSIILLNSGVYTFLSSTLKSLEEK
DHIHRALDKITDTLIHLMAKAGLTLQQQHQRLAQLLLILSHIRHM
SNKGMEHLYSMKYKNVVPLYDLLLEMLDAHRLHAGGSGASRVQAF
ADALDDKFLHDMLAEGLRYSVIREVLPTRRARTFDLEVEELHTLV
AEGVVVHNC
30R3-2 intein:
(SEQ ID NO: 5)
CLAEGTRIFDPVTGTTHRIEDVVDGRKPIHVVAAAKDGTLLARPV
VSWFDQGTRDVIGLRIAGGATVWATPDHKVLTEYGWRAAGELRKG
DRVAGPGGSGNSLALSLTADQMVSALLDAEPPILYSEYDPTSPFS
EASMMGLLTNLADRELVHMINWAKRVPGFVDLTLHDQAHLLECAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEELRYSVIREVLPTRRARTFDLEVEELHTLVA
EGVVVHNC
30R3-3 intein:
(SEQ ID NO: 6)
CLAEGTRIFDPVTGTTHRIEDVVDGRKPIHVVAAAKDGTLLARPV
VSWFDQGTRDVIGLRIAGGATVWATPDHKVLTEYGWRAAGELRKG
DRVAGPGGSGNSLALSLTADQMVSALLDAEPPIPYSEYDPTSPFS
EASMMGLLTNLADRELVHMINWAKRVPGFVDLTLHDQAHLLECAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEELRYSVIREVLPTRRARTFDLEVEELHTLVA
EGVVVHNC
37R3-1 intein:
(SEQ ID NO: 7)
CLAEGTRIFDPVTGTTHRIEDVVDGRKPIHVVAAAKDGTLLARPV
VSWFDQGTRDVIGLRIAGGATVWATPDHKVLTEYGWRAAGELRKG
DRVAGPGGSGNSLALSLTADQMVSALLDAEPPILYSEYNPTSPFS
EASMMGLLTNLADRELVHMINWAKRVPGFVDLTLHDQAHLLERAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEGLRYSVIREVLPTRRARTFDLEVEELHTLVA
EGVVVHNC
37R3-2 intein:
(SEQ ID NO: 8)
CLAEGTRIFDPVTGTTHRIEDVVDGRKPIHVVAAAKDGTLLARPV
VSWFDQGTRDVIGLRIAGGAIVWATPDHKVLTEYGWRAAGELRKG
DRVAGPGGSGNSLALSLTADQMVSALLDAEPPILYSEYDPTSPFS
EASMMGLLTNLADRELVHMINWAKRVPGFVDLTLHDQAHLLERAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEGLRYSVIREVLPTRRARTFDLEVEELHTLVA
EGVVVHNC
37R3-3 intein:
(SEQ ID NO: 9)
CLAEGTRIFDPVTGTTHRIEDVVDGRKPIHVVAVAKDGTLLARPV
VSWFDQGTRDVIGLRIAGGATVWATPDHKVLTEYGWRAAGELRKG
DRVAGPGGSGNSLALSLTADQMVSALLDAEPPILYSEYDPTSPFS
EASMMGLLTNLADRELVHMINWAKRVPGFVDLTLHDQAHLLERAW
LEILMIGLVWRSMEHPGKLLFAPNLLLDRNQGKCVEGMVEIFDML
LATSSRFRMMNLQGEEFVCLKSIILLNSGVYTFLSSTLKSLEEKD
HIHRALDKITDTLIHLMAKAGLTLQQQHQRLAQLLLILSHIRHMS
NKGMEHLYSMKYKNVVPLYDLLLEMLDAHRLHAGGSGASRVQAFA
DALDDKFLHDMLAEELRYSVIREVLPTRRARTFDLEVEELHTLVA
EGVVVHNC

The terms “glycan.” “sugar.” “carbohydrate.” or “saccharide.” are used interchangeably herein and refer to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. An exemplary monosaccharide is O-linked N-acetylglucosamine (O-GlcNAc). Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, respectively. If the monosaccharide contains an aldehyde, it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position cither above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, trehalose, and cellobiose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein. In some embodiments, the glycan is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, and derivatives thereof (e.g., N-acetylglucosamine, N-acetylgalactosamine, etc.).

The term “glycosylation,” as used herein, is the reaction in which a glycosyl donor is attached to a functional group of a glycosyl acceptor. In some embodiments, glycosylation may refer to an enzymatic process that attaches glycans to proteins. In some embodiments, glycosylation may refer to an enzymatic process that attaches glycans to other glycans already attached to a protein. In some embodiments, glycosylation is the transfer of saccharide moieties to other molecules. In some embodiments, glycosylation refers to the modification of amino acids, such as serine and threonine, through their hydroxyl groups on proteins.

The term “glycosidic bond,” as used herein, refers to a type of covalent bond that joins a carbohydrate to another group.

The term “linker,” as used herein, refers to a bond (e.g., a covalent bond), a chemical group, or a molecule linking two molecules or moieties, e.g., two domains of a mini OGA, such as a first portion and a second portion of the OGA stalk domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a linker joins a first portion and a second portion of a stalk domain of an OGA. In some embodiments, a linker comprises one or more repeats of the sequence GS. In certain embodiments, a linker comprises the sequence GSGSGSGSGSGSGSG (SEQ ID NO: 1).

The term “nuclear export sequence” or “NES” refers to an amino acid sequence that promotes transport of a protein out of the cell nucleus to the cytoplasm, for example, through the nuclear pore complex by nuclear transport. Nuclear export sequences are known in the art and would be apparent to the skilled artisan. For example, NES sequences are described in Xu, D. et al. Sequence and structural analyses of nuclear export signals in the NESdb database. Mol Biol. Cell. 2012, 23(18) 3677-3693, the contents of which are incorporated herein by reference for its disclosure of exemplary nuclear export sequences.

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences.

As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. In certain embodiments, an antibody is an antibody fragment. However, in some embodiments, an antibody is a Fab fragment, a F(ab′)2 fragment, a Fv fragment, or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known in the art. With respect to the heavy chain, in some embodiments, the heavy chain of an antibody described herein can be an alpha (α), delta (Δ), epsilon (ε), gamma (γ), or mu (μ) heavy chain. In some embodiments, the heavy chain of an antibody described herein comprises a human alpha (α), delta (Δ), epsilon (ε), gamma (γ), or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780. In some embodiments, the VH domain comprises an amino acid sequence that is at least 70%, at least 75%, at least 80%, at least 85%, at least 90%, at least 95%, at least 98%, or at least 99% identical to any of the variable chain constant regions. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecules includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger et al., Proceedings of the National Academy of Sciences 1993, 90, 6444; Poljak et al., Structure 1994, 2, 1121). In some embodiments, an antibody fragment is a nanobody.

A “nanobody,” as used herein, refers to a small protein recognition domain. A nanobody is the smallest antigen binding fragment or single variable domain derived from naturally occurring heavy chain antibody. Nanobodies are known to the person skilled in the art. They are derived from heavy chain only antibodies, seen in camelids (Hamers-Casterman et al. 1993; Desmyter et al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). A single variable domain heavy chain antibody may be referred to herein as a nanobody or a VHH antibody.

The terms “nucleic acid,” “polynucleotide,” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides, are linear molecules in which adjacent nucleotides are linked to each other via a phosphodiester linkage. In some embodiments, a “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single- and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or may include non-naturally occurring nucleotides or nucleosides. Furthermore, the terms “nucleic acid,” “DNA,” “RNA,” and/or similar terms include nucleic acid analogs, e.g., analogs having bonds other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, a polynucleotide encodes any of the glycosyl hydrolases provided herein.

The term “vector” refers to a polynucleotide comprising one or more recombinant polynucleotides of the present invention, e.g., those encoding a glycosyl hydrolase provided herein. Vectors include, but are not limited to, plasmids, viral vectors, cosmids, artificial chromosomes, and phagemids. Vectors are able to replicate in a host cell and are further characterized by one or more endonuclease restriction sites at which the vector may be cut and into which a desired nucleic acid sequence may be inserted. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase, or luciferase), and genes that visibly affect the phenotype of transformed or transfected cells, hosts, colonies, or plaques. Any vector suitable for the transformation of a host cell, (e.g., E. coli, mammalian cells such as CHO cell, insect cells, etc.) are embraced by the present invention, for example vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing gRNAs and/or proteins (e.g., those provided herein), transforming cells, and expressing/purifying recombinant proteins are well known in the art, and are provided by, for example, Green and Sambrook, Molecular Cloning: A Laboratory Manual (4th ed., Cold Spring Harbor Laboratory Press, Cold Spring Harbor, N.Y. (2012)).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may also be a “split protein.” A split protein, as used herein, refers to a protein that has been engineered to be expressed as two separate pieces. Together, the separate pieces may comprise the full-length protein, or they may comprise only a portion of the full-length protein.

The term “sample” may be used to generally refer to an amount or portion of something (e.g., a protein). A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. The “sample” may be any biological sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments a sample comprises cells, tissue, or cellular material (e.g., material derived from cells, such as a cell lysate, or fraction thereof). A sample of a cell line comprises a limited number of cells of that cell line. In some embodiments, a sample may be obtained from an individual who has been diagnosed with or is suspected of having a disease.

The term “pharmaceutical composition,” as used herein, refers to a composition that can be administered to a subject in the context of treatment of a disease or disorder. In some embodiments, a pharmaceutical composition comprises an active ingredient, e.g., any of the glycosyl hydrolases provided herein, such as OGA, and a pharmaceutically acceptable excipient. In some embodiments, the pharmaceutical composition comprises a ligand.

The terms “treatment,” “treat,” and “treating,” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “subject,” as used herein, refers to an individual organism. In some embodiments, the subject is a human. In some embodiments, the subject is a non-human mammal. In some embodiments, the subject is a non-human primate. In some embodiments, the subject is a rodent or a mouse. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The terms “condition,” “disease,” and “disorder” are used interchangeably.

The term “neurological disease” refers to any disease of the nervous system, including diseases that involve the central nervous system (brain, brainstem, and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorca; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing cyes-dancing fect syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; carly infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyncuritiformis; herpes zoster oticus; herpes zoster; Hirayama syndrome; HIV-associated dementia and neuropathy (sec also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelac; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's discase; paramyotonia congenita; parancoplastic diseases; paroxysmal attacks; Parry Romberg syndrome; Pelizacus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion discases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorca; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “cancer” refers to a group of diseases defined by the uncontrollable proliferation of abnormal cells. Examples of cancers include, but are not limited to, adenocarcinoma; anal cancer; appendix cancer; bladder cancer; breast cancer; brain cancer; cervical cancer; colorectal cancer; connective tissue cancer; esophageal cancer; ocular cancer; gall bladder cancer; gastric cancer; germ cell cancer; head and neck cancer; throat cancer; kidney cancer; liver cancer; lung cancer; muscle cancer; leukemia; bone cancer; ovarian cancer; pancreatic cancer; prostate cancer; and thyroid cancer.

The term “diabetes” refers to diabetes mellitus, which is a group of metabolic disorders defined by prolonged periods of high blood sugar levels. Diabetes may be type 1 diabetes, characterized by the failure of the pancreas to produce enough insulin. Diabetes may also be type 2 diabetes, characterized by the failure of the cells of the body to respond properly to the insulin produced by the pancreas. In some embodiments, diabetes is gestational diabetes.

The terms “effective amount” and “therapeutically effective amount” include an amount effective, at dosages and for periods of time necessary, to achieve a desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one at which any toxic or detrimental effects (e.g., side effects) of the inhibitor compound are outweighed by the therapeutically beneficial effects.

### DETAILED DESCRIPTION OF CERTAIN EMBODIMENTS

The aspects described herein are not limited to specific embodiments, methods, systems, or configurations, and as such can, of course, vary. The terminology used herein is for the purpose of describing particular aspects only and, unless specifically defined herein, is not intended to be limiting.

The present disclosure provides glycosyl hydrolases comprising an intein. Such glycosyl hydrolases allow for the spatial and temporal control of enzymatic activity, for example, by inhibiting the activity of the glycosyl hydrolase until the intein has been excised (e.g., upon being contacted with a ligand, such as a small molecule, in embodiments where the intein is a ligand-dependent intein). The present disclosure also provides pharmaceutical compositions comprising the glycosyl hydrolases disclosed herein, as well as polynucleotides, vectors, cells, systems, and kits. Methods of using the intein-containing glycosyl hydrolases are also provided herein. For example, the present disclosure provides methods of deglycosylating a target protein using the glycosyl hydrolases provided herein. Methods of treating a glycosylation-associated disease in a subject, as well as methods of sensitizing a cell to a desirable therapeutic outcome, are also provided herein.

### Glycosyl Hydrolases

In one aspect, the present disclosure provides glycosyl hydrolases comprising an intein. The intein may be inserted, for example, at a position within the glycosyl hydrolase. In some embodiments, the activity of the glycosyl hydrolase is disrupted by the intein and may be restored upon excision of the intein. Thus, for example, the glycosyl hydrolase may be unable to catalyze the cleavage of a glycosidic bond (e.g., to remove a sugar moiety attached to a post-translationally modified protein), until the intein has been excised from the glycosyl hydrolase. In some embodiments, the site of intein insertion is chosen such that the activity of the OGA is disrupted by the intein and the activity of the OGA is restored upon excision of the intein. In some embodiments, the intein is excised from the glycosyl hydrolase spontaneously. In some embodiments, the intein is excised from the glycosyl hydrolase upon a particular event, such as binding of a ligand (e.g., in embodiments in which the intein is a ligand-dependent intein).

Any glycosyl hydrolase may be used in the present invention, i.e., an intein may be inserted into any glycosyl hydrolase know in the art, or any glycosyl hydrolase that is discovered or characterized in the future. Numerous glycosyl hydrolases are known in the art, including several provided in the “Definitions” section above, and a person of ordinary skill in the art would be capable of determining additional glycosyl hydrolases suitable for use in the present invention. In some embodiments, a glycosyl hydrolase is an O-GlcNAcase (OGA). In some embodiments, a glycosyl hydrolase is an OGA variant, e.g., a variant comprising one or more amino acid truncations and/or other modifications relative to a wild-type OGA.

In some embodiments, a glycosyl hydrolase comprises a split OGA. Split OGAs are described, for example, in PCT publication WO 2022/076329 and Ge, Y. et al., “Target protein deglycosylation in living cells by a nanobody-fused split O-GlcNAcase.” Nat. Chem. Biol. 2021, 17, 593, each of which is incorporated herein by reference. A split OGA may comprise a first piece comprising a catalytic domain and a second piece comprising a stalk domain. In some embodiments, the catalytic domain comprises a truncated catalytic domain. In some embodiments, the stalk domain comprises a truncated stalk domain. In certain embodiments, the catalytic domain comprises a truncated catalytic domain, and the stalk domain comprises a truncated stalk domain.

In some embodiments, a glycosyl hydrolase comprises mini OGA. Mini OGA comprises a truncation of the C-terminal HAT domain relative to wild type OGA. Mini OGA also comprises a peptide linker that is inserted within the stalk domain of a wild type OGA. For example, the mini OGA may comprise the structure NH₂-[catalytic domain]-[first portion of stalk domain]-[linker]-[second portion of stalk domain]-COOH. The linker joining the first portion and the second portion of the stalk domain may be any linker known in the art or provided herein. In some embodiments, the linker comprises one or more amino acids. In some embodiments, the linker is a peptide linker. In some embodiments, the linker comprises one or more repeats of the sequence GS. In certain embodiments, the linker inserted between the first portion and the second portion of the stalk domain, for example, comprises the sequence

(SEQ ID NO: 1)
GSGSGSGSGSGSGSG.

In certain embodiments, a glycosyl hydrolase is an OGA. In some embodiments, the glycosyl hydrolase comprises the amino acid sequence of SEQ ID NO: 107, provided below. In some embodiments, a glycosyl hydrolase comprises an amino acid sequence at least 80%, at least 85%, at least 90%, at least 95%, or at least 99% identical to the amino acid sequence of SEQ ID NO: 107.

Any intein described herein or known in the art may be used in the glycosyl hydrolases provided herein. In some embodiments, the intein is a ligand-dependent intein. A ligand-dependent intein, for example, may be excised from the glycosyl hydrolase upon ligand binding. Ligand-dependent inteins that recognize various types of ligands are known in the art, and additional ligand-dependent inteins may be engineered by a person of ordinary skill in the art. In some embodiments, the ligand is a small molecule, a peptide, a protein, an amino acid, a polynucleotide, or a nucleic acid. In certain embodiments, the ligand is a small molecule (e.g., a cell permeable and/or nontoxic small molecule). In certain embodiments, the ligand is 4-hydroxytamoxifen (4HT). The intein may be inserted at any position within the glycosyl hydrolase. In some embodiments, the intein is inserted at a position that leads to little or no activity when inserted and in which activity is restored activity when the intein is excised.

In some embodiments, upon excision, the intein leaves a cysteine residue. Thus, if the intein is inserted such that it replaces a cysteine, the glycosyl hydrolase, upon intein excision, will be unmodified as compared to the original protein. If the intein replaces any other amino acid, the glycosyl hydrolase, upon intein excision, will contain a cysteine in place of the amino acid that was replaced. In some embodiments, the intein does not replace an amino acid residue in a glycosyl hydrolase, but is inserted into the glycosyl hydrolase (e.g., in addition to the amino acid residues of the glycosyl hydrolase). In such embodiments, upon excision, the protein will comprise an additional cysteine residue. While the presence of an additional cysteine residue (or the substitution of a residue for a cysteine upon excision) is unlikely to affect the function of the glycosyl hydrolase, in some embodiments where the intein does not replace a cysteine, the intein replaces an alanine, serine, or threonine amino acid, as these residues are similar in size and/or polarity to cysteine.

In some embodiments, the intein is inserted at or replaces a cysteine within the glycosyl hydrolase. In some embodiments, the intein is inserted at or replaces a cysteine selected from the group consisting of C62, C166, C181, C220, C316, C596, C631, and C663 in SEQ ID NO: 107. In certain embodiments, the intein is inserted at or replaces amino acid position C181 in SEQ ID NO: 107.

The intein that is inserted into the protein can be any ligand-dependent intein, e.g., those described herein. For example, in some embodiments, the intein that is inserted into the protein comprises, in part or in whole, the amino acid sequence of any one of SEQ ID NOs: 2-9, or an amino acid sequence that is at least 80%, at least 85%, at least 90%, at least 95%, or at least 99% identical to the amino acid sequence of any one of SEQ ID NO: 2-9.

In certain embodiments, the glycosyl hydrolase comprising the intein comprises the amino acid sequence of SEQ ID NO: 108, or an amino acid sequence at least 80%, at least 85%, at least 90%, at least 95%, or at least 99% identical to the amino acid sequence of SEQ ID NO: 108.

In certain embodiments, the glycosyl hydrolase comprising the intein+D174N comprises the amino acid sequence of SEQ ID NO: 109, or an amino acid sequence at least 80%, at least 85%, at least 90%, at least 95%, or at least 99% identical to the amino acid sequence of SEQ ID NO: 109.

In certain embodiments, the glycosyl hydrolase comprising the intein and an NLS comprises the amino acid sequence of SEQ ID NO: 110, or an amino acid sequence at least 80%, at least 85%, at least 90%, at least 95%, or at least 99% identical to the amino acid sequence of SEQ ID NO: 110.

In certain embodiments, the glycosyl hydrolase comprising the intein and an NES comprises the amino acid sequence of SEQ ID NO: 111, or an amino acid sequence at least 80%, at least 85%, at least 90%, at least 95%, or at least 99% identical to the amino acid sequence of SEQ ID NO: 111.

It will be appreciated by those of skill in the art that other ligand-dependent inteins are also suitable and useful in connection with the glycosyl hydrolases and methods provided herein. For example, some aspects of this invention provide glycosyl hydrolases comprising ligand-dependent inteins that comprise a ligand-binding domain of a hormone-binding protein, e.g., of an androgen receptor, an estrogen receptor, an ecdysone receptor, a glucocorticoid receptor, a mineralocorticoid receptor, a progesterone receptor, a retinoic acid receptor, or a thyroid hormone receptor protein. Ligand-binding domains of hormone-binding receptors, inducible fusion proteins comprising such ligand-binding domains, and methods for the generation of such fusion proteins are known to those of skill in the art (see, e.g., Becker, D., Hollenberg, S., and Ricciardi, R. (1989). “Fusion of adenovirus E1A to the glucocorticoid receptor by high-resolution deletion cloning creates a hormonally inducible viral transactivator.” Mol. Cell. Biol. 9, 3878-3887; Bochmelt, G., Walker, A., Kabrun, N., Mellitzer, G., Boug, H., Zenke, M., and Enrictto, P. J. (1992). “Hormone-regulated v-rel estrogen receptor fusion protein: reversible induction of cell transformation and cellular gene expression.” EMBO J 11, 4641-4652; Braselmann, S., Graninger, P., and Busslinger, M. (1993). “A selective transcriptional induction system for mammalian cells based on Gal4-estrogen receptor fusion proteins.” Proc Natl Acad Sci USA 90, 1657-1661; Furga G, Busslinger M (1992). “Identification of Fos target genes by the use of selective induction systems.” J. Cell Sci. Suppl 16,97-109; Christopherson, K. S., Mark, M. R., Bajaj, V., and Godowski, P. J. (1992). “Ecdysteroid-dependent regulation of genes in mammalian cells by a Drosophila ecdysone receptor and chimeric transactivators.” Proc Natl Acad Sci USA 89, 6314-8; Eilers, M., Picard, D., Yamamoto, K., and Bishop, J. (1989). “Chimacras of Myc oncoprotein and steroid receptors cause hormone-dependent transformation of cells.” Nature 340, 66-68; Fankhauser, C. P., Briand, P. A., and Picard, D. (1994). “The hormone binding domain of the mineralocorticoid receptor can regulate heterologous activities in cis.” Biochem Biophys Res Commun 200, 195-201; Godowski, P. J., Picard, D., and Yamamoto, K. R. (1988). “Signal transduction and transcriptional regulation by glucocorticoid receptor-LexA fusion proteins.” Science 241, 812-816; Kellendonk, C., Tronche, F., Monaghan, A., Angrand, P., Stewart, F., and Schütz, G. (1996). “Regulation of Cre recombinase activity by the synthetic steroid RU486”. Nuc. Acids Res. 24, 1404-1411; Lec, J. W., Moore, D. D., and Heyman, R. A. (1994). “A chimeric thyroid hormone receptor constitutively bound to DNA requires retinoid X receptor for hormone-dependent transcriptional activation in yeast.” Mol Endocrinol 8, 1245-1252; No, D., Yao, T. P., and Evans, R. M. (1996). “Ecdysone-inducible gene expression in mammalian cells and transgenic mice.” Proc Natl Acad Sci USA 93, 3346-3351; and Smith, D., Mason, C., Jones, E., and Old, R. (1994). “Expression of a dominant negative retinoic acid receptor g in Xenopus embryos leads to partial resistance to retinoic acid.” Roux's Arch. Dev. Biol. 203, 254-265; all of which are incorporated herein by reference in their entirety). Additional ligand-binding domains useful for the generation of ligand-dependent inteins as provided herein will be apparent to those of skill in the art, and the invention is not limited in this respect.

Additional exemplary inteins, ligand-binding domains, and ligands suitable for use in the glycosyl hydrolases disclosed herein are described in International Patent Application, PCT/US2012/028435, entitled “Small Molecule-Dependent Inteins and Uses Thereof,” filed Mar. 9, 2012, and published as WO 2012/125445 on Sep. 20, 2012, the entire contents of which are incorporated herein by reference. Other suitable inteins, ligand-binding domains, and ligands will be apparent to the skilled artisan based on this disclosure.

The glycosyl hydrolases provided herein may also be further modified in order to target them to particular subcellular locations, or to particular proteins of interest. In some embodiments, the glycosyl hydrolases are fused to a nuclear localization sequence (NLS). The NLS may facilitate targeting of the glycosyl hydrolase to the nucleus of a cell and/or selective spatial deglycosylation in the nucleus. Exemplary NLSs include, for example, those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which is incorporated herein by reference. Examples of NLSs that may be used in conjunction with the glycosyl hydrolases provided herein include, without limitation, the sequences MAPKKKRKVGIHRGVP (SEQ ID NO: 10), PKKKRKV (SEQ ID NO: 11), MKRTADGSEFESPKKKRKV (SEQ ID NO: 12), MDSLLMNRRKFLYQFKNVRWAKGRRETYLC (SEQ ID NO: 13), AVKRPAATKKAGQAKKKKLD (SEQ ID NO: 14), MSRRRKANPTKLSENAKKLAKEVEN (SEQ ID NO: 15), PAAKRVKLD (SEQ ID NO: 16), KLKIKRPVK (SEQ ID NO: 17), VSRKRPRP (SEQ ID NO: 18), EGAPPAKRAR (SEQ ID NO: 19), PPQPKKKPLDGE (SEQ ID NO: 20), SGGSKRTADGSEFEPKKKRKV (SEQ ID NO: 21), and KRTADGSEFESPKKKRKV (SEQ ID NO: 22).

In some embodiments, the glycosyl hydrolase is fused to a nuclear export sequence (NES). The NES may facilitate targeting of the glycosyl hydrolase to the cytoplasm of a cell and/or selective spatial deglycosylation in the cytoplasm of a cell. Exemplary NESs include, for example, those described in Xu, D. et al. “Sequence and structural analyses of nuclear export signals in the NESdb database.” Mol. Biol. Cell. 2012, 23(18), 3677-3693; Fung, H. Y. J. et al. “Structural determinants of nuclear export signal orientation in binding to exportin CRM1.” eLife. 2015, 4:c10034; and Kosugi, S. et al. “Nuclear Export Signal Consensus Sequences Defined Using a Localization-based Yeast Selection System.” Traffic. 2008, 9(12), 2053-2062, each of which is incorporated herein by reference. Examples of NESs that may be used in conjunction with the glycosyl hydrolases disclosed herein include, without limitation, the sequences: MEELSQALASSFSV (SEQ ID NO: 23), PLQLPPLERLTL (SEQ ID NO: 24), NELALKLAGLDI (SEQ ID NO: 25), ERFEMFRELNEALEL (SEQ ID NO: 26), DHAEKVAEKLEALSV (SEQ ID NO: 27), QLVEELLKIICAFQL (SEQ ID NO: 28), TNLEALQKKLEELEL (SEQ ID NO: 29), DVKEEMTSALATMRV (SEQ ID NO: 30), STNGSLAAEFRHLQL (SEQ ID NO: 31), PSVQELTEQIHRLLM (SEQ ID NO: 32), MNFKELKDFLKELNI (SEQ ID NO: 33), ENFEILMKLKESLEL (SEQ ID NO: 34), FETVYELTKMCTIR (SEQ ID NO:35), SGKASSSLGLQDFDL (SEQ ID NO:36), PKYSDIDVDGLCSEL (SEQ ID NO: 37), VDLACTPTDVRDVDI (SEQ ID NO: 38), YGEKTTQRDLTELEI (SEQ ID NO: 39), RRIYDITNVLEGIGL (SEQ ID NO: 40), AKIIPYSGLLLVITV (SEQ ID NO: 41), LRSEEVHWLHVDMGV (SEQ ID NO: 42), LQSEEVHWLHLDMGV (SEQ ID NO: 43), LQVRKYSLDLASLIL (SEQ ID NO: 44), AGVEAIIRILQQLLF (SEQ ID NO: 45), TGVEALIRILQQLLF (SEQ ID NO: 46), IVLNQLCVRFFGLDL (SEQ ID NO:47), SLGGFEITPPVVLRL, EAIQDLCLAVEEVSL (SEQ ID NO: 49), DELLQVLRMMVGVNI (SEQ ID NO: 50), SVMLAVQEGIDLLTF (SEQ ID NO: 51), LSSHFQELSI (SEQ ID NO: 52), QSTHVDIRTLEDLLM (SEQ ID NO: 53), ESSAEDLRTLQQLFL (SEQ ID NO: 54), EFSLPTHHTVRLIRV (SEQ ID NO: 55), MSSGYYLGEILRLAL (SEQ ID NO: 56), DTVLDILRDFFELRL (SEQ ID NO: 57), NSVNEILSEFYYVRL (SEQ ID NO: 58), CAFLSVKKQFEELTL (SEQ ID NO: 59), ISPEHVIQALESLGF (SEQ ID NO: 60), AHWMRQLVSFQKLKL (SEQ ID NO: 61), ATRELDELMASLSDF (SEQ ID NO: 62), YQNIELITFINALKL (SEQ ID NO: 63), FNATAVVRHMRKLQL (SEQ ID NO: 64), SGIFGLVTNLEELEV (SEQ ID NO: 65), EESYTLNSDLARLGV (SEQ ID NO: 66), EESYDLTSHLARLGV (SEQ ID NO: 67), GIQQAHAEQLANMRI (SEQ ID NO: 68), DVKEEMTSALATMRV (SEQ IS NO: 30), AAEPVILDLRDLFQL (SEQ ID NO: 69), MEGCVSNLMV (SEQ ID NO: 70), EGCVSNLMV (SEQ ID NO: 71), DMDFLRNLFSQTLSL (SEQ ID NO: 72), EQLLEIVHDLENLSL (SEQ ID NO: 73), NVMKYFTDLFDYLPL (SEQ ID NO: 74), KVYPIILRLGSNLSL (SEQ ID NO: 75), YAGFSLPHAILRIDL (SEQ ID NO: 76), EIVRDIKEKLCYVAL (SEQ ID NO: 77), EAINKLESNLRELQI (SEQ ID NO: 78), EAINKLENNLRELQI (SEQ ID NO: 79), SDQKQEQLLLKKMYL (SEQ ID NO: 80), KQVLWDRTFSLFQQL (SEQ ID NO: 81), AQLQNLTKRIDSLPL (SEQ ID NO: 82), NDENEHQLSLRTVSL (SEQ ID NO: 83), ISFTEFVKVLEKVDV (SEQ ID NO: 84), MESAITLWQFLLQL (SEQ ID NO: 85), VPKELMQQIENFEKI (SEQ ID NO: 86), QARFILEKIDGKIII (SEQ ID NO: 87), QVKFIKMIIEKELTV (SEQ ID NO: 88), NHRMKNLREISQLGI (SEQ ID NO: 89), NHRVKKLNEISKLGI (SEQ ID NO: 90), TEKHLQKYLRQDLRL (SEQ ID NO: 91), RQERKRPLLDLHIEL (SEQ ID NO: 92), ANMRIQDLKVSLKPL (SEQ ID NO: 93), ATMRVDYEQIKIKKI (SEQ ID NO: 94), LQGEEFVCLKSIILL (SEQ ID NO: 95), THYGQKAILFLPLPV (SEQ ID NO: 96), PSAHEITGLADSLQL (SEQ ID NO: 97), VRLHDVLHSDKKLTL (SEQ ID NO: 98), LINRNGELKLANFGL (SEQ DI NO: 99), and LEPLKKLECLKSLDL (SEQ ID NO: 100).

In some embodiments, the glycosyl hydrolases provided herein are fused to a targeting molecule. In certain embodiments, the targeting molecule facilitates targeting of the glycosyl hydrolase to a particular protein target. In certain embodiments, the targeting molecule facilitates targeting of the glycosyl hydrolase to a particular cell type. In some embodiments, the targeting molecule is an antibody, or a fragment thereof (e.g., an antibody that recognizes a particular target protein, or a cell surface protein on a particular cell type). In certain embodiments, the targeting molecule is a nanobody (e.g., a nanobody that recognizes a particular target protein, or a cell surface protein on a particular cell type). In certain embodiments, the targeting molecule is an antigen binding fragment.

### Pharmaceutical Compositions, Polynucleotides, Vectors, Cells, and Kits

In other aspects, the present disclosure provides pharmaceutical compositions comprising any of the glycosyl hydrolases disclosed herein and a pharmaceutically acceptable excipient. The term “pharmaceutical composition”, as used herein, refers to a composition formulated for pharmaceutical use. In some embodiments, the pharmaceutical composition further comprises a pharmaceutically acceptable excipient. In some embodiments, the pharmaceutical composition comprises additional agents (e.g., for specific delivery, increasing half-life, or other therapeutic compounds).

As used here, the term “pharmaceutically-acceptable excipient” or “pharmaceutically-acceptable carrier” means a pharmaceutically-acceptable material, composition, or vehicle, such as a liquid or solid filler, diluent, excipient, carrier, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue, or portion of the body). A pharmaceutically acceptable excipient or carrier is “acceptable” in the sense of being compatible with the other ingredients of the formulation and not injurious to the tissue of the subject (e.g., physiologically compatible, sterile, physiologic pH, etc.). Some examples of materials that can serve as pharmaceutically-acceptable excipients include: (1) sugars, such as lactose, glucose, and sucrose; (2) starches, such as corn starch and potato starch; (3) cellulose, and its derivatives, such as sodium carboxymethyl cellulose, methylcellulose, ethyl cellulose, microcrystalline cellulose, and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; (22) bulking agents, such as polypeptides and amino acids; (23) serum component, such as serum albumin, HDL, and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. Terms such as “excipient, “carrier, “pharmaceutically acceptable carrier, or the like are used interchangeably herein.

In some embodiments, a pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, a pharmaceutical composition described herein is administered locally to a diseased site (e.g., a tumor site). In some embodiments, a pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, a pharmaceutical composition described herein is delivered in a controlled release system. In one embodiment, a pump may be used (sec, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. Sec, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105. Other controlled release systems are discussed, for example, in Langer, supra.

In some embodiments, a pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to case pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a pharmaceutical composition is to be administered by infusion, it can be dispensed with an infusion bottle containing sterile pharmaceutical grade water or saline. Where a pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's solution, or Hank's solution. In addition, a pharmaceutical composition can be in solid forms and re-dissolved or suspended immediately prior to use. Lyophilized forms are also contemplated.

A pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Active ingredients can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-diolcoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged, for example, as a unit dose. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier or vehicle.

Further, a pharmaceutical composition can be provided as a pharmaceutical kit comprising (a) a container containing an active ingredient of the invention in lyophilized form and (b) a second container containing a pharmaceutically acceptable diluent (e.g., sterile water) for injection. The pharmaceutically acceptable diluent can be used for reconstitution or dilution of the lyophilized active ingredient of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a glycosyl hydrolase of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

In other aspects, the present disclosure provides polynucleotides encoding any of the glycosyl hydrolases disclosed herein.

In other aspects, the present disclosure provides vectors comprising any of the polynucleotides encoding any of the glycosyl hydrolases disclosed herein.

In other aspects, the present disclosure provides cells comprising any of the glycosyl hydrolases, polynucleotides, or vectors disclosed herein.

In other aspects, the present disclosure provides kits. In some embodiments, the kits comprise any of the glycosyl hydrolases provided herein. In some embodiments, the kits comprise any of the polynucleotides encoding glycosyl hydrolases provided herein. In some embodiments, the kits comprise any of the vectors comprising polynucleotides encoding glycosyl hydrolases provided herein. In some embodiments, the kits comprise a ligand specific for an intein.

Any of the kits described herein may include one or more containers housing components for performing the methods described herein, and optionally instructions for uses. Any of the kits described herein may further comprise components needed for performing the methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. As used herein, “promoted” includes all methods of doing business including methods of education, scientific inquiry, academic research, and any advertising or other promotional activity including written, oral and electronic communication of any form, associated with the disclosure. Additionally, the kits may include other components depending on the specific application, as described herein.

The kits may contain any one or more of the components described herein in one or more containers. The kits may have a variety of forms, such as a blister pouch, a shrink-wrapped pouch, a vacuum scalable pouch, a scalable thermoformed tray, or a similar pouch or tray form, with the accessories loosely packed within the pouch, one or more tubes, containers, a box, or a bag. The kits may also include other components, depending on the specific application, for example, containers, cell media, salts, buffers, reagents, etc.

### Methods of Using Glycosyl Hydrolases

The present disclosure provides methods for removing a glycan from, or deglycosylating, a target protein using any of the glycosyl hydrolases provided herein, as well as uses thereof for studying the effects of glycosylation on protein function in one or more cells. For example, the glycosyl hydrolases can be OGA. Also provided are methods of treating a glycosylation-associated disease in a subject. Also provided are methods of using a glycosyl hydrolase, for example, OGA, in the treatment of treating a glycosylation-associated disease in a subject. In addition, methods are provided of reducing the drug resistance in a cell by modulating the glycosylation state of one or more proteins in the cell related to drug resistance.

In one aspect, the present disclosure provides methods of deglycosylating a target protein. In some embodiments, the methods comprise (i) contacting a target protein containing a sugar moiety with any of the glycosyl hydrolases provided herein, and (ii) contacting the glycosyl hydrolase with a ligand, thereby excising the intein from the glycosyl hydrolase and restoring its activity. Any target protein that has been post-translationally modified with one or more target moieties may be deglycosylated using the methods provided herein. In some embodiments, the sugar moiety is removed from the target protein upon restoration of the activity of the glycosyl hydrolase. In certain embodiments, the sugar moiety is an O-linked N-acetyl glucosamine. In certain embodiments, the O-linked N-acetyl glucosamine is removed from a serine or threonine residue of the target protein. In some embodiments, the method is performed in a cell (e.g. a cancer cell). In some embodiments, the cell is in a subject. In certain embodiments, the subject is a human.

In another aspect, the present disclosure provides methods of studying the effects of glycosylation on protein function in one or more cells using any of the glycosyl hydrolases provided herein.

In another aspect, the present disclosure provides methods of treating a glycosylation-associated disease in a subject. In some embodiments, the methods comprise (i) administering to the subject a therapeutically effective amount of any of the glycosyl hydrolases provided herein, and (ii) contacting the glycosyl hydrolase with a ligand, thereby excising the intein from the glycosyl hydrolase and restoring its activity. In some embodiments, the disease is a neurodegenerative disease. In certain embodiments, the neurodegenerative disease is selected from the group consisting of Parkinson's disease, Huntington's disease, Alzheimer's disease, dementia, and multiple system atrophy. In some embodiments, the neurodegenerative disease is Parkinson's disease. In some embodiments, the neurodegenerative disease is Huntington's disease. In some embodiments, the disease is proliferative disease. In some embodiments, the disease is cancer. In some embodiments, the disease is metabolic disease. In some embodiments, the disease is diabetes.

In another aspect, the methods provided herein are used for reducing drug resistance in a cell by modulating the glycosylation state of one or more proteins in the cell using any of the glycosyl hydrolases provided herein. In some aspects, the methods provided herein are used for sensitizing a cell to a desirable therapeutic outcome by modulating the glycosylation state of one or more proteins in the cell using any of the glycosyl hydrolases provided herein. In some embodiments, the cell is a cancer cell.

### EXAMPLES

The function and advantage of these and other embodiments of the present invention will be more fully understood from the Examples below. The following Examples are intended to illustrate the benefits of the present invention and to describe particular embodiments but are not intended to exemplify the full scope of the invention. Accordingly, it will be understood that the Examples are not meant to limit the scope of the invention.

### Example 1: Small Molecule-Activated O-GlcNAcase for Spatiotemporal Removal of O-GlcNAc in Live Cells

### Design, Engineering, and Optimization of OGA-Intein Fusions.

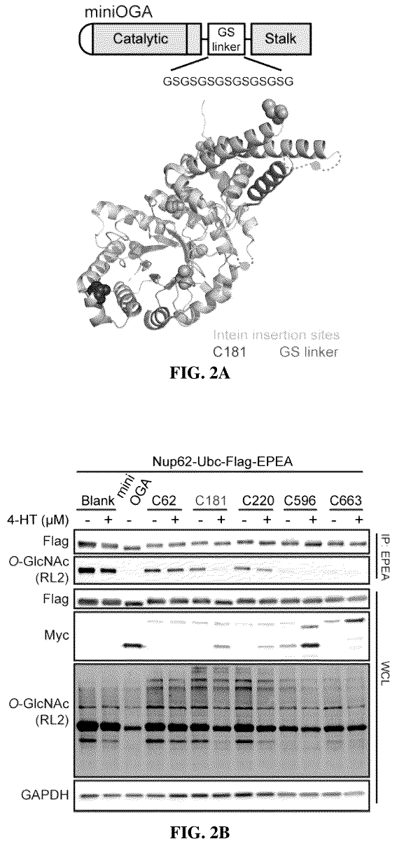

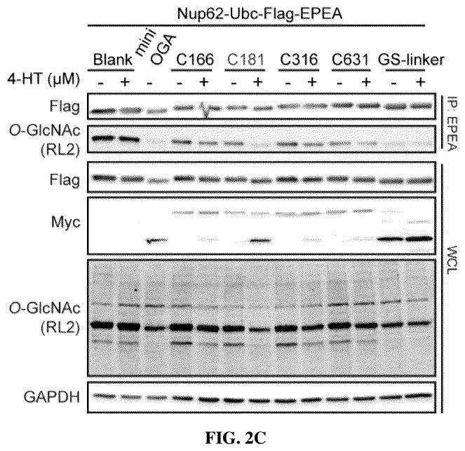

OGA is a multi-domain hydrolase responsible for O-GlcNAc removal in mammalian cells. Based on the reported crystal structures of OGA (27-29), the essential domains of the long splice variant of OGA were identified, and it was confirmed that the HAT domain is not required for its deglycosidase activity in cells (13). In addition, replacement of the disordered region between the catalytic domain and the stalk domain with a glycine-serine flexible linker (GS-linker) maintained its activity in live cells in the absence of the C-terminal HAT domain (13, 28). This variant, termed miniOGA, was selected as the initial template for engineering with an intein due to its smaller size and simpler structure (FIG. 2A). A screen was set up to determine the potential sites for intein incorporation, aiming to obtain an OGA-intein fusion that was inactive until splicing occurs after adding 4-HT. Inserting this evolved 4-HT-responsive intein into a site that abolishes miniOGA activity until protein splicing has taken place could result in a conditionally active OGA for O-GlcNAc removal under the control of 4-HT. As intein splicing would leave behind a single cysteine residue (22), and to minimize alterations on OGA protein sequence, cysteine residues across different structural domains and the GS-linker at miniOGA were selected as the candidate insertion sites, thus generating OGA-intein fusion constructs for nine positions in total (C62, C166, C181, C220, C316, C596, C631, C663, and the GS-linker) (FIG. 2A). The nine OGA-intein variants were transiently expressed in HEK293T cells together with a model glycoprotein Nup62. Cells were treated with or without 1 μM 4-HT for 24 hours after transfection. OGA-intein variants were then detected by the Myc tag to verify the expected splicing, and their enzymatic activities were evaluated by detection of O-GlcNAc on the substrate Nup62 following immunoprecipitation.

The intein insertion site was important for affecting miniOGA's activity before and after adding 4-HT. It was determined that the OGA-intein(C181) variant exhibited little activity in the absence of 4-HT and high deglycosylation activity in the presence of 4-HT after yielding the active spliced product, miniOGA (FIGS. 2B, 2C). By contrast, miniOGA bearing the intein at C596 or in the GS-linker spontaneously spliced even in the absence of 4-HT. The variant with the intein at C663 remained active and was unable to release the intein on 4-HT treatment, implying that the intein at the C663 site distal to the catalytic domain can neither affect OGA's activity nor respond to 4-HT. Apart from these three constructs, intein insertion at the other six selected Cys sites all resulted in a loss of OGA's activity. Except for the C181 candidate, other variants generated little or undetectable spliced product after incubation with 4-HT (FIGS. 2B, 2C). Without wishing to be bound by this theory, it is possible that these variants have difficulties in initiating splicing upon the binding of 4-HT or re-folding properly for stable spliced product. Accordingly, the activatable OGA-intein(C181) variant was investigated in subsequent experiments for small molecule-triggered spatiotemporal deglycosylation.

### 4-HT Triggered OGA-Intein Activation is Dose- and Time-Dependent.

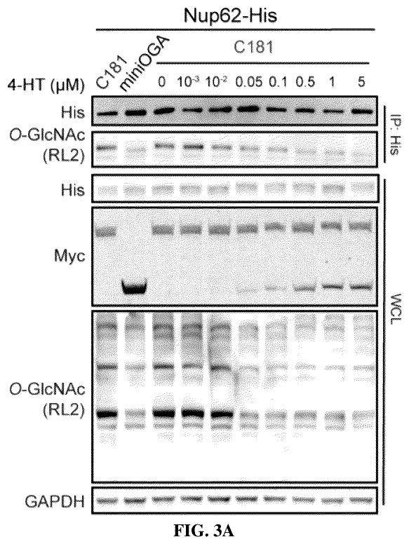

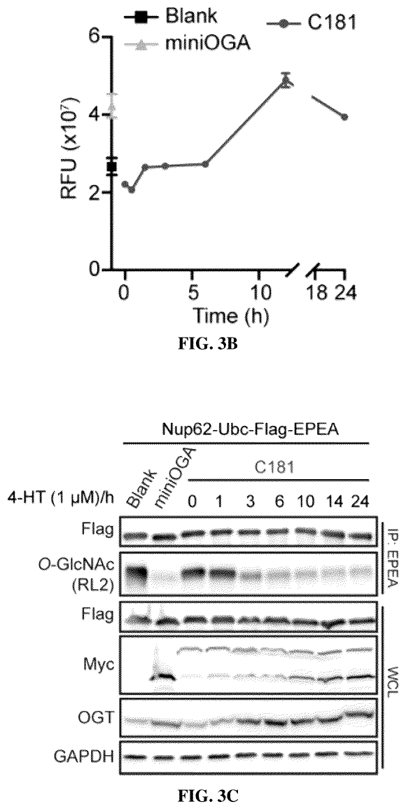

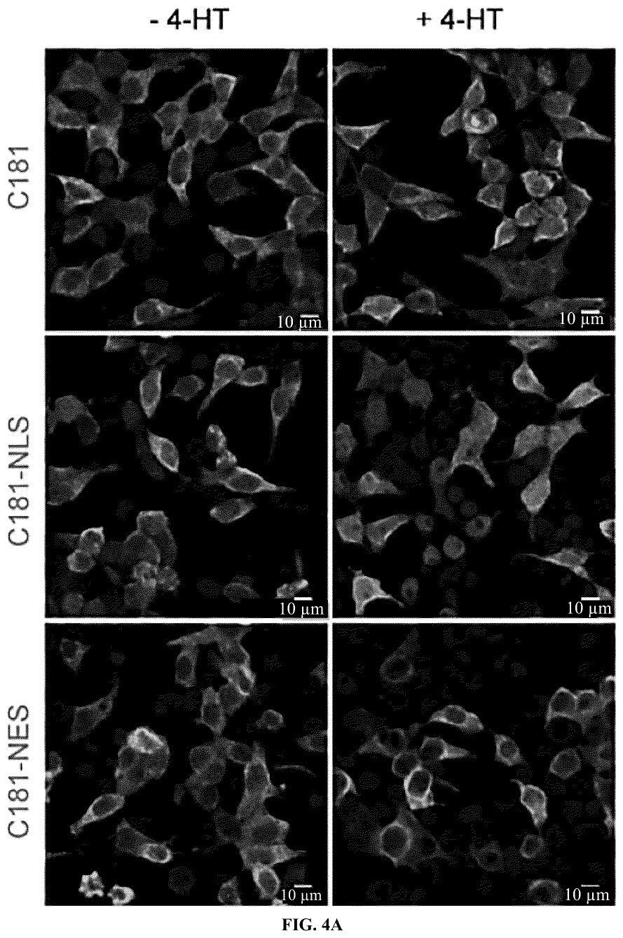

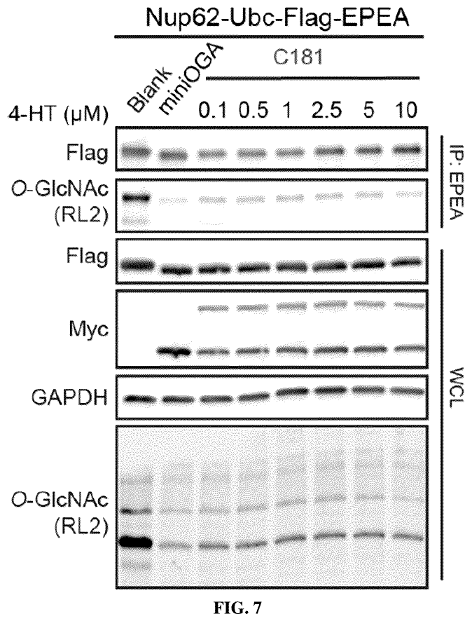

Next, the activation efficacy under different conditions was characterized. 4-HT-triggered OGA activation, and the corresponding deglycosylation on Nup62, was tested over concentrations ranging from 1 nM to 5 μM (FIG. 3A). A dose-dependent reduction of O-GlcNAc on Nup62 and an increase of spliced OGA-intein fusion as a function of 4-HT concentration from 1 nM to 5 μM after 15 hours was observed. The spliced product was detectable with an induction of 50 nM 4-HT, while considerable reduction of O-GlcNAc on Nup62 occurs with 100 nM 4-HT treatment. After extending the incubation time to 24 hours, different concentrations of 4-HT resulted in the splicing of C181 reaching a plateau, and the cellular hypo-O-GlcNAcylation state persisted through 24 hours (FIG. 7). It was possible that the incubation time of 4-HT could be important for the process of OGA-intein activation and deglycosylation.

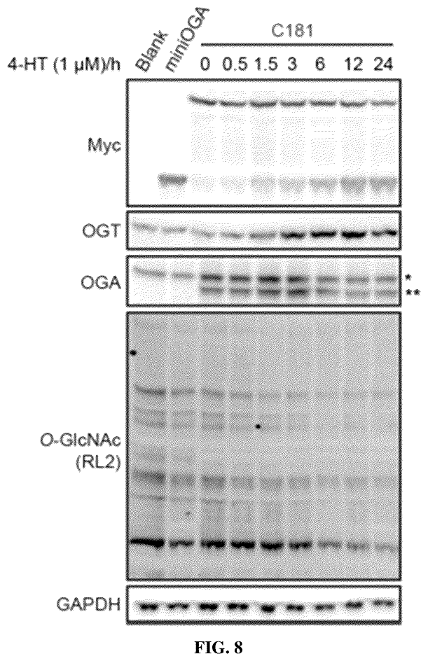

Therefore, it was evaluated whether 4-HT induced OGA-intein activation was a time-dependent process. An in vitro OGA activity assay (30) was used to measure the overall hexoaminidase activity from cell samples treated with 4-HT over a series of time points (FIGS. 3B and 8). Both the spliced product (FIG. 8) and the hydrolase activity (FIG. 3B) were elevated as a function of 4-HT incubation time. It was noted that OGA activity mildly decreased after activation for 24 hours. As the in vitro OGA activity assay reads the combined contributions from both exogenously expressed OGA-intein and endogenous OGA, it was possible that the cellular response to maintain O-GlcNAc homeostasis may offset the overall effects of OGA-intein activation after longer treatment, such as through regulation of OGT and OGA levels by detained intron splicing (6) and intron retention (5). Therefore, it was next assessed when the endogenous O-GlcNAc machinery responded to the perturbations on O-GlcNAc levels by 4-HT treatment.

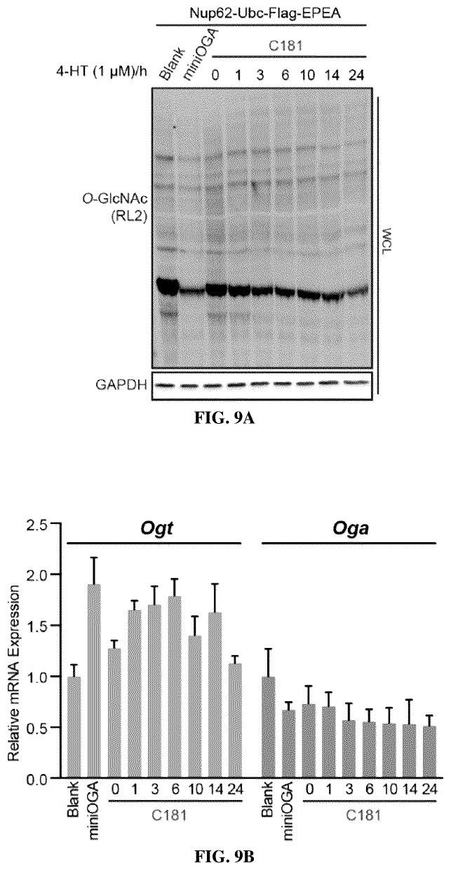

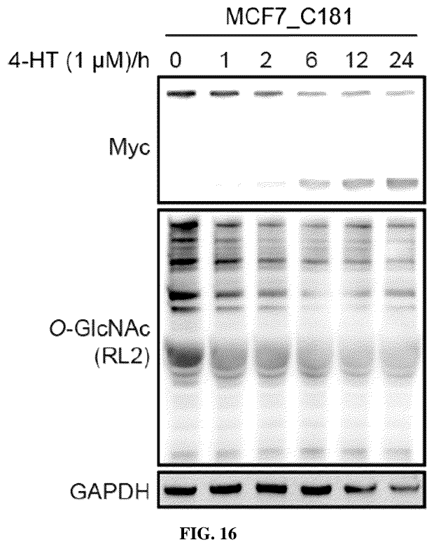

A time-course activation experiment was performed using 1 μM 4-HT when co-expressed with OGA-intein(C181) with the model substrate Nup62. Similar to the results of the in vitro OGA activity assay, a gradual accumulation of active spliced product was observed together with increased incubation time starting from 1 hour. Similarly, significant deglycosylation on Nup62 occurred when cells were treated with 1 μM 4-HT for 3 hours, which lagged slightly behind OGA-intein activation. At the same time point, endogenous OGT protein levels also exhibited an obvious compensatory increase (FIGS. 3C and 8), and a minor decrease occurs on endogenous OGA proteins (FIG. 8). Since the transcripts typically responded faster than protein levels, mRNA levels of endogenous OGT and OGA were recorded together with the time-dependent OGA-intein activation (FIGS. 9A-9B). OGT mRNA levels increased as early as 1 hour after activation, while OGA mRNA level decreased in a time-dependent manner that corresponded to OGA-intein activation and thereby decreased O-GlcNAcylation, though the overall change is subtle. Similar to the in vitro OGA activity assay, OGT mRNA recovered back to basal levels after 24-hour activation, potentially due to the large accumulation of OGT protein. This suggests that cells responded to the OGA-intein activation primarily through upregulating both protein and mRNA levels of OGT within a short response time window, which is analogous to the time-dependent upregulation of these species after OGT inhibition (3). Activation in MCF-7 cells stably expressing OGA-intein(C181) similarly confirmed the time-dependent deglycosylation effects (FIG. 16). Taken together, it was demonstrated that 4-HT-triggered OGA-intein activation is dose- and time-dependent while inducing regulatory feedback to maintain cellular O-GlcNAc homeostasis.

### Spatial Deglycosylation by Subcellular Localized OGA-Intein(C181).

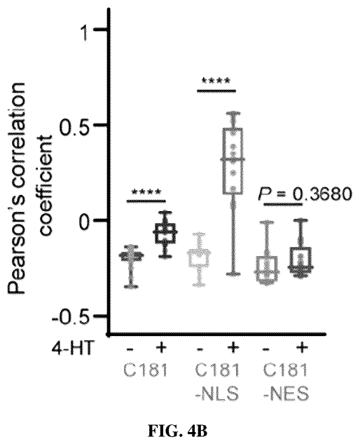

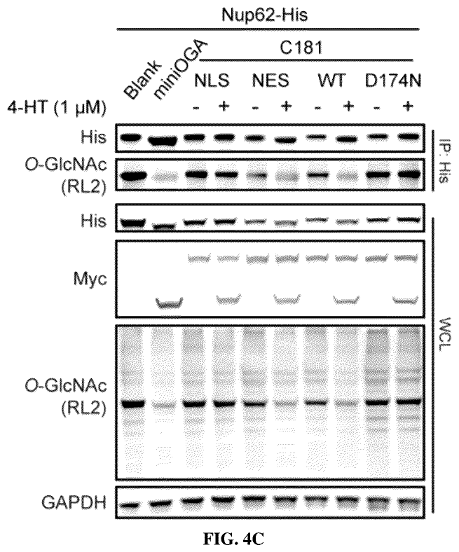

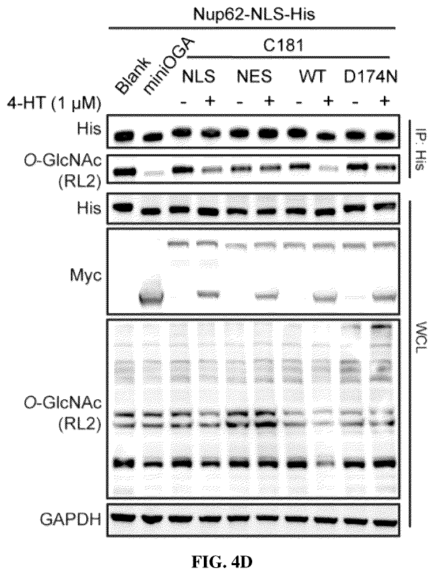

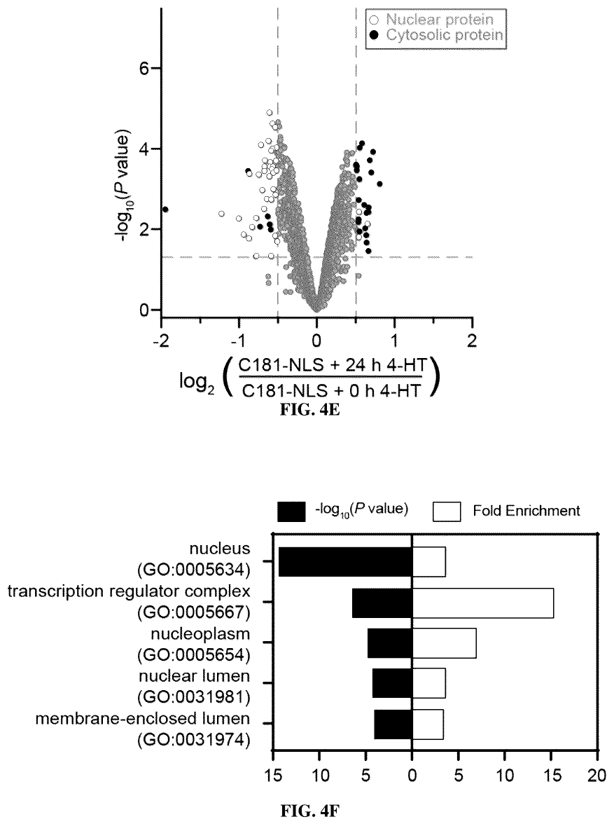

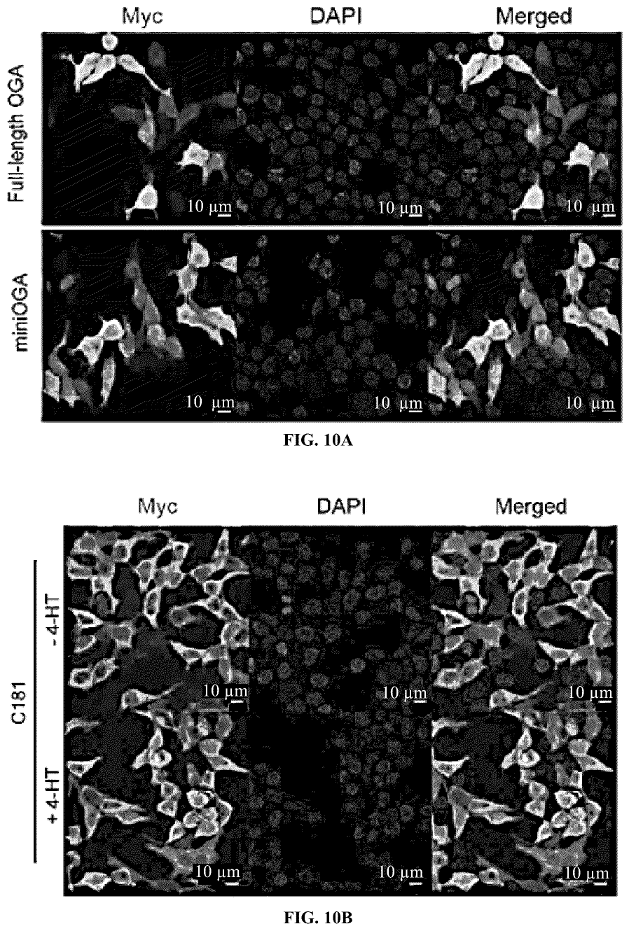

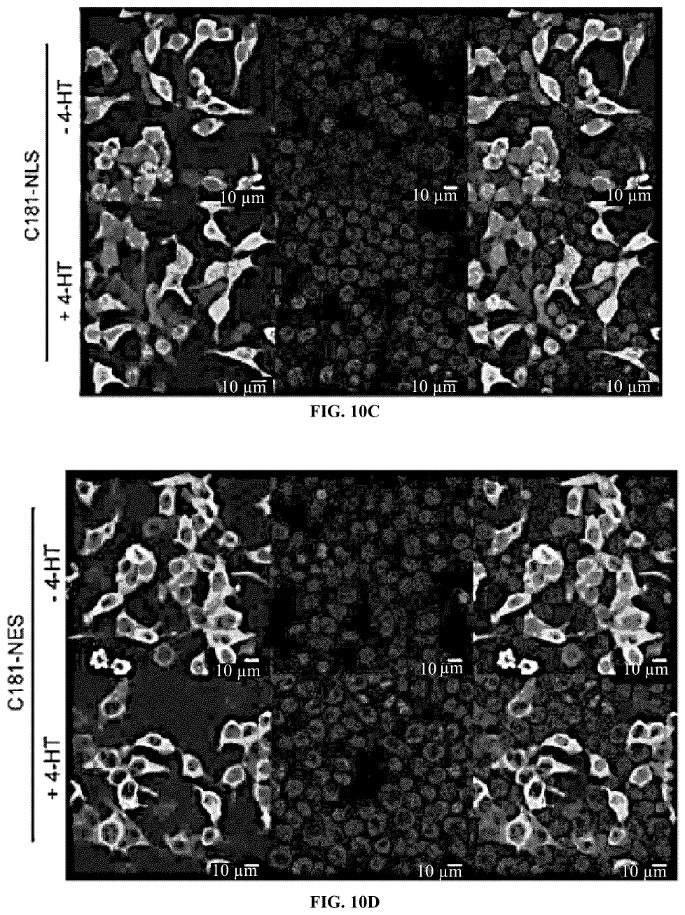

The O-GlcNAc modification is responsive to environmental changes (1, 31), and also possesses distinct compartment-specific dynamics in the cell (2, 32). Correspondingly, OGA is a nucleocytoplasmic enzyme (33), whereas the long-spliced OGT primarily localizes in the nucleus (34). Therefore, the 4-HT-triggered OGA-intein activation strategy was extended for controllable spatial specific deglycosylation. The distribution of OGA-intein(C181) with or without the treatment of 4-HT was assessed. OGA-intein(C181) is mainly localized in the cytoplasm before activation and was partially transported to the nucleus after activation, consistent with the distribution of mini-OGA (FIG. 4A and FIG. 10A). The OGA-intein(C181) was appended to a nuclear-localization sequence (NLS) (35) or a nuclear-export sequence (NES) (36) for nuclear and cytosolic targeting, respectively. Both fusions exhibited a primarily cytosolic distribution before activation. After activation by 4-HT, the spliced OGA-intein(C181)-NLS (C181-NLS) partially translocated into the nucleus, whereas OGA-intein(C181)-NES (C181-NES) remained in the cytoplasm (FIG. 4A and FIGS. 10A-10D). (C181)-NLS may not pass through the nuclear membrane due to the large protein size before activation but can translocate to the nucleus on 4-HT induction to a smaller spliced product.

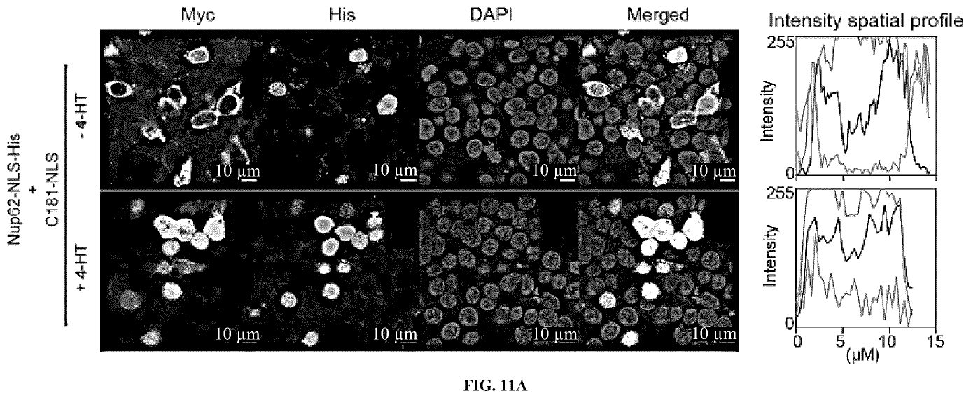

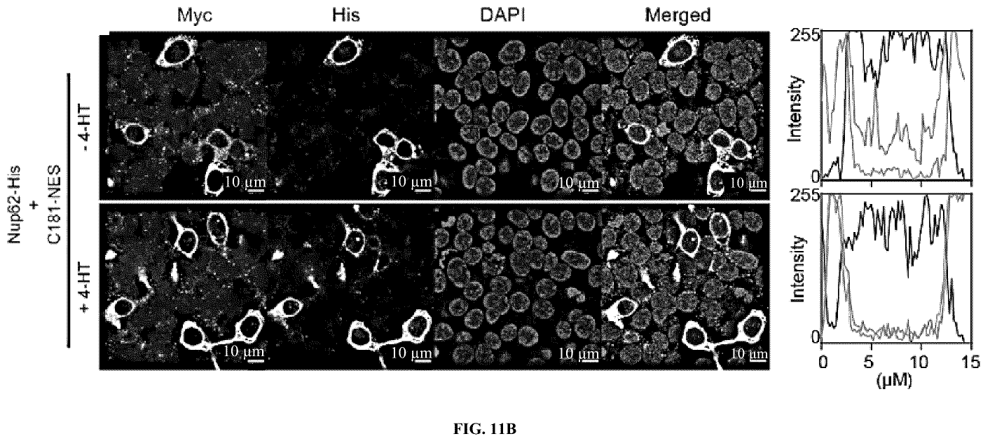

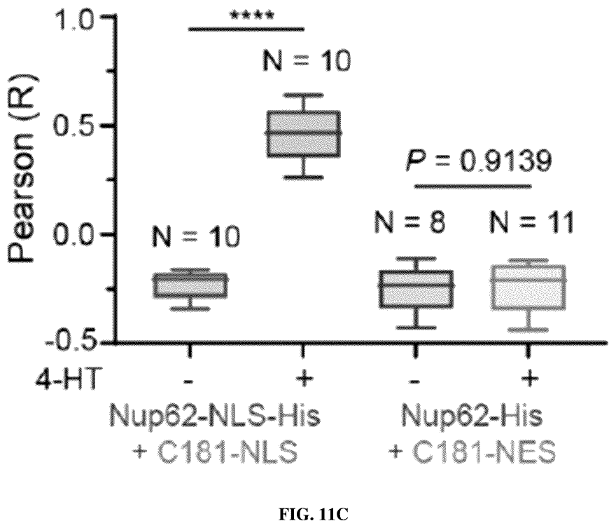

Next, it was assessed whether these OGA-intein(C181) variants enable selective spatial deglycosylation in live cells. As O-GlcNAc affects the subcellular localization of many proteins (37), Nup62 was used as a model cytoplasmic substrate, which was previously primarily localized in the cytoplasm when transiently overexpressed in HEK293T cells independent of the O-GlcNAcylation state (13) (FIGS. 11A, 11B). Nup62 was co-expressed with three OGA-intein(C181) variants (C181-WT, C181-NLS, and C181-NES) in HEK293T cells, and Nup62 was enriched for O-GlcNAc measurement. After 4-HT-triggered activation, only the cytoplasmic C181-WT and C181-NES, but not the nuclear C181-NLS, removed O-GlcNAc from Nup62 (FIG. 4C). Next, Nup62 was localized to the nucleus by attachment to an NLS tag (Nup62-NLS), and a similar coexpression and 4-HT-triggered activation was performed with OGA-intein(C181) variants. Localization of Nup62-NLS in the nucleus allowed 4-HT-induced deglycosylation only by the nuclear C181-WT and C181-NLS, but not the cytoplasmic C181-NES (FIG. 4D). The fluorescent intensity of OGA-intein variants also show a strong correlation to the localization of Nup62, indicating the desired subcellular distribution (FIGS. 11A-C).

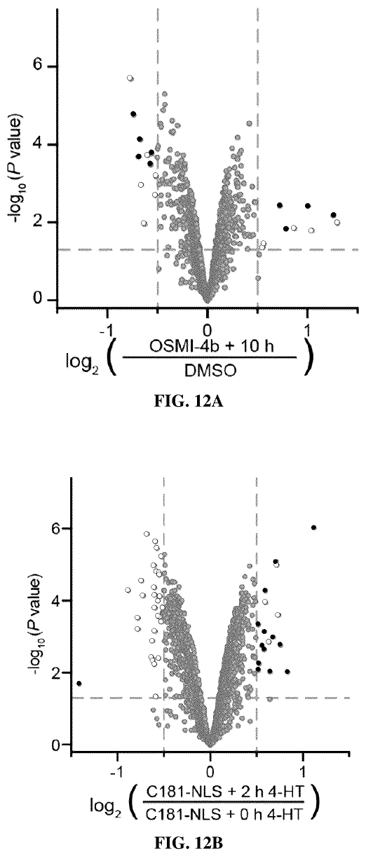

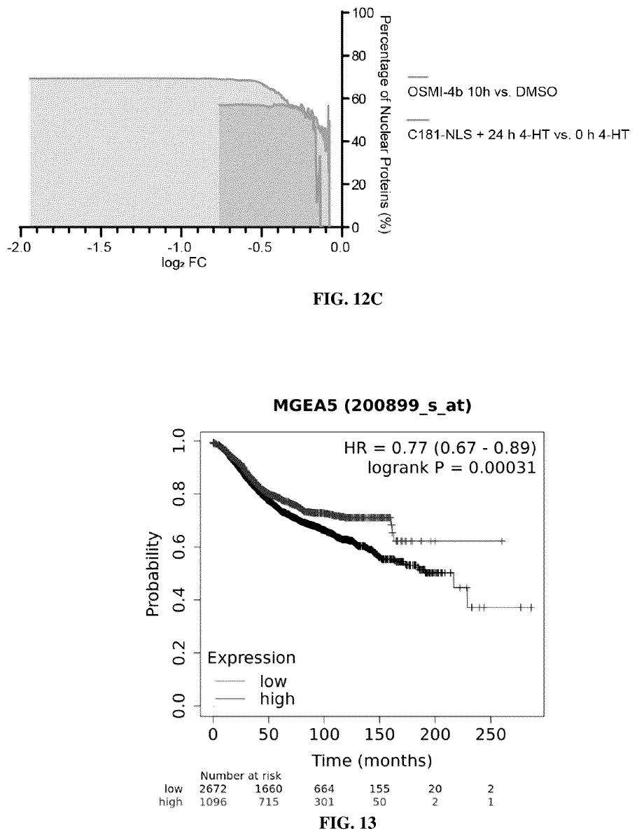

To further assess the spatial deglycosylation mediated by these subcellularly localized OGA-intein variants, quantitative proteomics was performed following enrichment for O-GlcNAc (38) on cell lysates after expression of C181-NLS with 4-HT treatment for 2 hours and 24 hours, respectively, or the OGT inhibitor OSMI-4b (3) for comparison (FIG. 4E and FIGS. 12A, 12B, 12C). Treatment with OSMI-4b for 10 hours was relatively indiscriminate and led to mild reduction of O-GlcNAc on both nuclear and cytoplasmic proteins (FIG. 12A). In contrast, treatment with 4-HT for 2 hours was sufficient to specifically reduce O-GlcNAc on nuclear O-GlcNAc proteins, and these differences became more pronounced after 24 hours (FIG. 4E, FIGS. 12B and 12C). Gene Ontology analysis also revealed that proteins that were significantly deglycosylated upon 4-HT-triggered OGA-intein activation were overrepresented in the nucleus and nuclear relevant complex (FIG. 4F), in agreement with the evaluation of Nup62 after expression of OGA-intein(C181) in nuclear or cytoplasmic compartments. These observations demonstrated that localization of OGA-intein(C181) to a specific subcellular compartment enabled selective spatial deglycosylation without perturbing glycoproteins in other compartments, thus providing spatial control on O-Glc-NAcylation inside cells.

### 4-HT and OGA Activation Synergistically Decrease the Viability of Breast Cancer Cells.

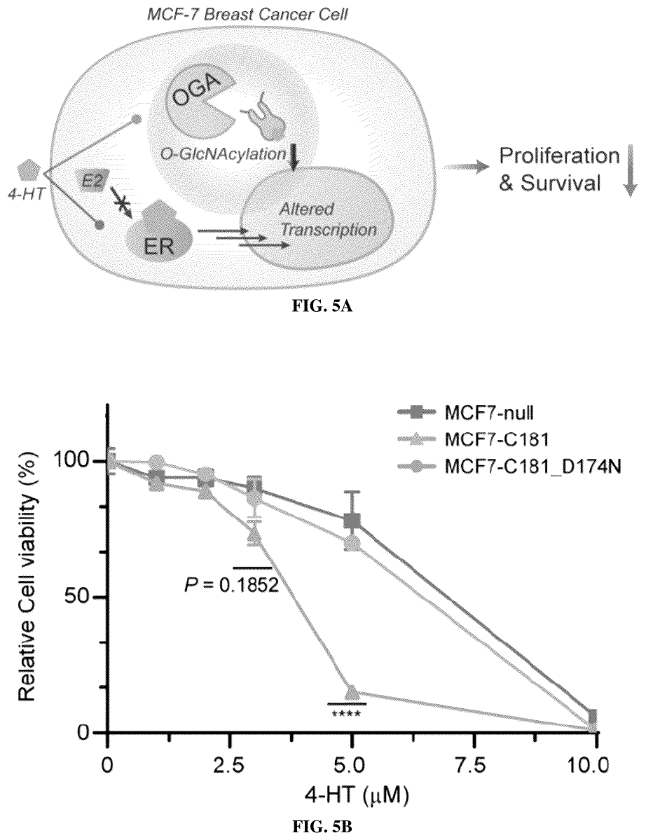

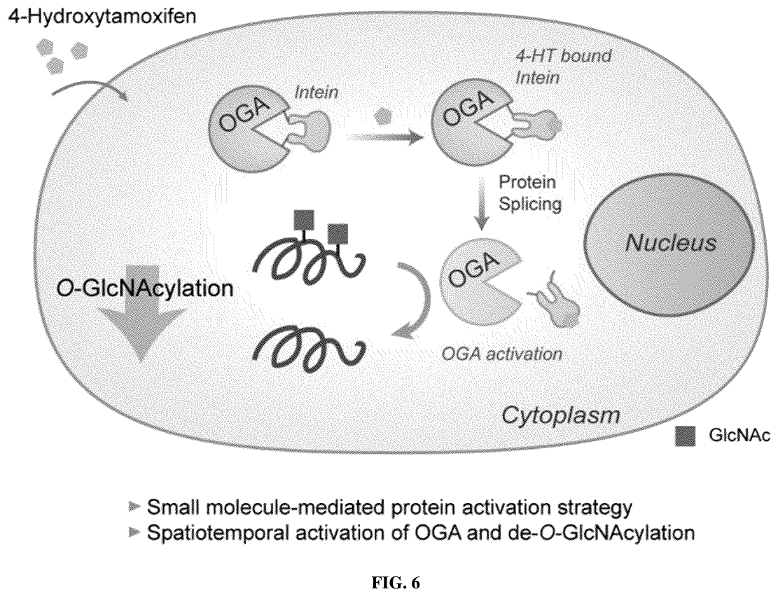

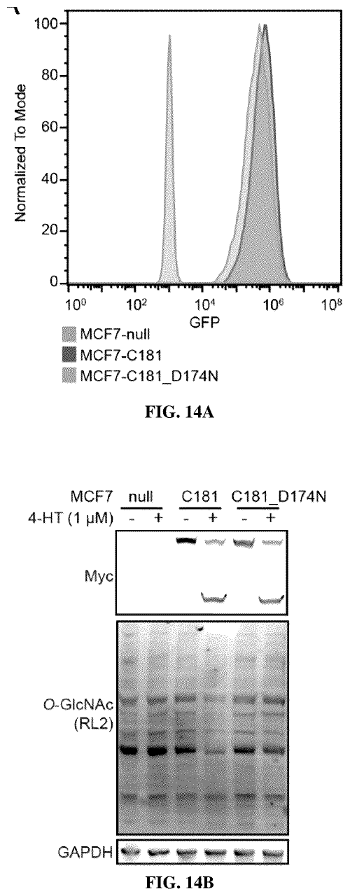

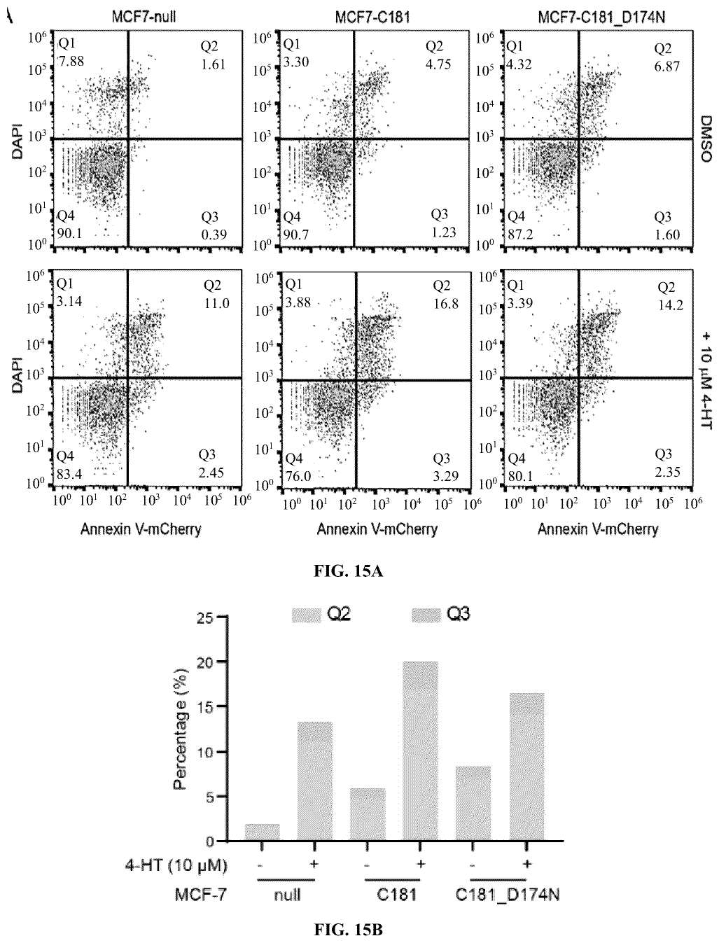

In addition to serving as an activator for intein splicing, 4-HT is also known as the active metabolite of tamoxifen, an FDA-approved drug for treating breast cancer patients. Breast cancer cells also have elevated OGT expression (39) and decreased levels of OGA (40, 41), resulting in elevated O-GlcNAcylation in the proteome. OGT is usually required for tumor growth and metastasis (39), and low OGA expression is correlated with poor survival in breast cancer patients (FIG. 13). The ER positive breast cancer-derived MCF-7 cell line is sensitive to tamoxifen and 4-HT, resulting in suppression of cell proliferation, yet drug resistance occurs after long-term treatment (42). Previous studies also show evidence that upregulation of O-GlcNAcylation protects MCF-7 cells from tamoxifen-induced cell death (43). To determine if reduction of O-GlcNAc sensitized MCF-7 cells to 4-HT treatment, it was assessed whether 4-HT induced inhibition of breast cancer cell proliferation and OGA-intein activation worked synergistically to accelerate cell death (FIG. 5A). Two MCF-7 cell lines were generated that stably expressed active OGA-intein(C181) and its catalytically dead mutant (D174N), respectively. A reduced global O-GlcNAcylation level was only observed after adding 4-HT to MCF-7 with active OGA-inteinC181 (MCF7-C181) instead of MCF-7 with the inactive mutant (MCF7-C181_D174N, FIG. 14A). Otherwise, the two OGA variants expressed similarly and successfully underwent splicing upon the addition of 4-HT (FIG. 14B). Next, these two cell lines were treated with 4-HT at increasing concentrations, and cell viability was measured after 48 hours. Almost complete cell survival inhibition was achieved when 5 μM 4-HT was applied to MCF7-C181 cells, whereas higher concentration of 4-HT was required for suppressing the survival of regular MCF-7 cells and MCF7-C181_D174N cells, indicating that deglycosylation of MCF-7 sensitized cells to 4-HT treatment (FIG. 5B). Given that 4-HT promoted MCF-7 cell death through apoptosis (43), the apoptosis of these cells under 4-HT treatment was further analyzed through flow cytometry analyses following Annexin V and DAPI staining. Indeed, 4-HT promoted apoptosis in all three MCF-7 cell lines according to considerably increased percentage of Annexin V positive cells after 4-HT incubation. Compared to 4-HT-induced apoptosis on MCF7-C181_D174N generating 6.09% apoptotic cells, 4-HT-treated MCF7-C181 cells displayed 6.84% apoptotic cells, suggesting that apoptosis may not be the primary inhibitory mechanism exacerbated by 4-HT induced O-GlcNAc reduction (FIGS. 15A-B). These data indicate that 4-HT may serve as a dual-functional modulator. Further, removal of O-Glc-NAc in MCF-7 cells sensitized cells to 4-HT treatment, which may mitigate the risk of acquiring drug resistance or enhance the therapeutic window in additional cell types.

A small molecule-triggered OGA activation strategy in live cells for controllable spatiotemporal removal of O-GlcNAc was developed. By integration of an evolved intein that splices in response to 4-HT to OGA, a set of incorporation sites was screened for the highest activation-to-background ratio, and an optimal OGA-intein fusion, OGA-intein(C181), was obtained. Activation of OGA-intein(C181) and removal of O-GlcNAc allowed precise regulation in a dose- and time-dependent fashion. In addition, localization of OGA-intein(C181) to different subcellular compartments enabled spatial control over deglycosylation, which was validated on both a specific substrate (Nup62) and the broader glycoprotcome. Finally, it was demonstrated that 4-HT served as a dual-functional modulator in MCF-7 cells stably expressing OGA-intein(C181) by antagonizing ER and activating OGA-intein(C181), which accelerated cell death and implied that modulating O-GlcNAc may sensitize cells to desirable therapeutic outcomes. In combination with the recently reported OGT activation method16, these tools could facilitate complementary profiling and functional studies of O-GlcNAcylated proteins under a desired condition.

The O-GlcNAc cycling enzymes, OGT and OGA, dynamically govern the changes of O-GlcNAcylation, responding to environmental cues. In contrast to the wide application of chemical and genetic inhibition of OGT and OGA, strategies for modulation of O-GlcNAc with spatial and temporal resolution are still underexplored, limiting the study of O-Glc-NAc functions. The controllable O-GlcNAc modulation approach holds the potential to investigate the dynamics of O-GlcNAc on different substrates and locations in the cell by offering an initial time point to track the corresponding feedback and recovery of O-GlcNAcylation. The rapid cellular response to activation of the OGA-intein, with an increase of OGT at both transcription and translation levels was observed, which could also provide insights into the maintenance of O-GlcNAc homeostasis within cells. Although the inactive OGA-intein fusion was usually overexpressed in cells described above, the active spliced product can be precisely produced by the addition of 4-HT, inducing substantial deglycosylation with a small protein amount (FIG. 3). Due to the modularity of this design, this activation approach can be further equipped with targeting modules like nanobodies for higher substrate selectivity (44). These tools could facilitate complementary profiling and functional studies of O-GlcNAcylated proteins under a desired condition, in combination with the recently reported OGT activation method (16). However, this activation strategy is irreversible, leading to persistent OGA activation after adding 4-HT. The activation additionally takes nearly 3 hours to achieve substantial O-GlcNAc removal, yet the internal responses for O-GlcNAc homeostasis usually occur on the timescale of minutes (5). Future OGT or OGA could be engineered with reversible ON and OFF switches as well, or could be engineered to have faster activation kinetics (45). For example, tools like bioorthogonal cleavage chemistry (15) and ligand-controlled protein conformational switch (46) could be utilized.

4-HT has passed phase 2 clinical trials and is thus safe and applicable to animal models. It has also been widely implemented for generation of inducible gene knock-out systems on both cultured cells and animal models (47), such as the inducible OGT knockout MEF cell line (17), indicating its limited side effects in vivo. Therefore, compared to photo-activation strategies (16), the 4-HT triggered activation of OGA-intein(C181) may be compatible with more complex in vivo settings. OGA-intein(C181) can therefore be used for high spatial and temporal removal of O-GlcNAc in desired cell types or tissues. Triggering OGA activation for reduced O-GlcNAcylation can have potential therapeutic benefits by synergizing with other treatments (48). For example, reduction of O-GlcNAc through inhibiting OGT was recently reported to promote ferroptosis in U2OS cancer cells (49). Recent advances in mRNA delivery (50) provide the opportunity to implement this selectively activatable OGA for assisting O-GlcNAc modulation in vivo. This work regarding the modulation of O-GlcNAc and its spatiotemporal relationship to cellular processes, and the connection of O-GlcNAc to biological function, could eventually lead to new therapeutic targets in the future.

### Methods

Cell culture and transfection. HEK293T and MCF-7 cells were cultured in Dulbecco's Modified Eagle Medium (DMEM) supplemented with penicillin (50 μg/mL) and streptomycin (50 μg/mL) along with 10% (v/v) FBS. Transfections of all plasmids in this study were performed using TransIT-PRO® (Mirus Bio, MIR5740) according to the manufacturer's instructions.

Plasmids and subcloning. The 4-HT-dependent evolved intein fragment was amplified from pKMD106e-intein-Cas9(S219) (Addgene #64190) and inserted into indicated sites on miniOGA plasmid. NLS (nuclear localization signal) or NES (nuclear export signal) were added in the C-terminus of Nup62 or OGA-intein variants to generate subcellular localized constructs. OGA-intein(C181) sequence and its inactive form were subcloned into lentiCas9-EGFP vector by replacing Cas9 (Addgene #63592) for the generation of stable cell lines. Unless otherwise noted, other constructs used in this study were from previous studies: PCT publication WO 2022/076329 and (13) Ge, Y. et al., Target protein deglycosylation in living cells by a nanobody-fused split O-GlcNAcase. Nat. Chem. Biol. 2021, 17, 593, each of which is incorporated herein by reference.

myc-OGA(GS-544)
(SEQ ID NO: 107)
MEQKLISEEDLAIAMVQKESQATLEERESELSSNPAASAGASLEP
PAAPAPGEDNPAGAGGAAVAGAAGGARRFLCGVVEGFYGRPWVME
QRKELFRRLQKWELNTYLYAPKDDYKHRMFWREMYSVEEAEQLMT
LISAAREYEIEFIYAISPGLDITFSNPKEVSTLKRKLDQVSQFGC
RSFALLFDDIDHNMCAADKEVESSFAHAQVSITNEIYQYLGEPET
FLFCPTEYCGTFCYPNVSQSPYLRTVGEKLLPGIEVLWTGPKVVS
KEIPVESIEEVSKIIKRAPVIWDNIHANDYDQKRLFLGPYKGRST
ELIPRLKGVLTNPNCEFEANYVAIHTLATWYKSNMNGVRKDVVMT
DSEDSTVSIQIKLENEGSDEDIETDVLYSPQMALKLALTEWLQEF
GVPHQYSSRGSGSGSGSGSGSGSGEKPLYTAEPVTLEDLQLLADL
FYLPYEHGPKGAQMLREFQWLRANSSVVSVNCKGKDSEKIEEWRS
RAAKFEEMCGLVMGMFTRLSNCANRTILYDMYSYVWDIKSIMSMV
KSFVQWLGCRSHSSAQFLIGDQEPWAFRGGLAGEFQRLLPIDGAN
DLFFQPP*
myc-OGA(1-180)-C-intein-OGA(182, GS-544)
(SEQ ID NO: 108)
MEQKLISEEDLAIAMVQKESQATLEERESELSSNPAASAGASLEP
PAAPAPGEDNPAGAGGAAVAGAAGGARRFLCGVVEGFYGRPWVME
QRKELFRRLQKWELNTYLYAPKDDYKHRMFWREMYSVEEAEQLMT
LISAAREYEIEFIYAISPGLDITFSNPKEVSTLKRKLDQVSQFGC
RSFALLFDDIDHNMCLAEGTRIFDPVTGTTHRIEDVVDGRKPIHV
VAAAKDGTLLARPVVSWFDQGTRDVIGLRIAGGAIVWATPDHKVL
TEYGWRAAGELRKGDRVAGPGGSGNSLALSLTADQMVSALLDAEP
PILYSEYDPTSPFSEASMMGLLTNLADRELVHMINWAKRVPGFV
DLTLHDQAHLLERAWLEILMIGLVWRSMEHPGKLLFAPNLLLDR
NQGKCVEGMVEIFDMLLATSSRFRMMNLQGEEFVCLKSIILLNSG
VYTFLSSTLKSLEEKDHIHRALDKITDTLIHLMAKAGLTLQQQHQ
RLAQLLLILSHIRHMSNKGMEHLYSMKYKNVVPLYDLLLEMLDAH
RLHAGGSGASRVQAFADALDDKFLHDMLAEGLRYSVIREVLPTRR
ARTFDLEVEELHTLVAEGVVVHNCAADKEVESSFAHAQVSITNEI
YQYLGEPETFLFCPTEYCGTFCYPNVSQSPYLRTVGEKLLPGIEV
LWTGPKVVSKEIPVESIEEVSKIIKRAPVIWDNIHANDYDQKRLF
LGPYKGRSTELIPRLKGVLTNPNCEFEANYVAIHTLATWYKSNMN
GVRKDVVMTDSEDSTVSIQIKLENEGSDEDIETDVLYSPQMALKL
ALTEWLQEFGVPHQYSSRGSGSGSGSGSGSGSGEKPLYTAEPVTL
EDLQLLADLFYLPYEHGPKGAQMLREFQWLRANSSVVSVNCKGKD
SEKIEEWRSRAAKFEEMCGLVMGMFTRLSNCANRTILYDMYSYVW
DIKSIMSMVKSFVQWLGCRSHSSAQFLIGDQEPWAFRGGLAGEFQ
RLLPIDGANDLFFQPP*
myc-OGA(1-180)-C-intein-OGA(182, GS-544) + D174N
(SEQ ID NO: 109)
MEQKLISEEDLAIAMVQKESQATLEERESELSSNPAASAGASLEP
PAAPAPGEDNPAGAGGAAVAGAAGGARRFLCGVVEGFYGRPWVME
QRKELFRRLQKWELNTYLYAPKDDYKHRMFWREMYSVEEAEQLMT
LISAAREYEIEFIYAISPGLDITESNPKEVSTLKRKLDQVSQFGC
RSFALLFNDIDHNMCLAEGTRIFDPVTGTTHRIEDVVDGRKPIHV
VAAAKDGTLLARPVVSWFDQGTRDVIGLRIAGGAIVWATPDHKVL
TEYGWRAAGELRKGDRVAGPGGSGNSLALSLTADQMVSALLDAEP
PILYSEYDPTSPFSEASMMGLLTNLADRELVHMINWAKRVPGFVD
LTLHDQAHLLERAWLEILMIGLVWRSMEHPGKLLFAPNLLLDRN
QGKCVEGMVEIFDMLLATSSRFRMMNLQGEEFVCLKSIILLNSGV
YTFLSSTLKSLEEKDHIHRALDKITDTLIHLMAKAGLTLQQQHQR
LAQLLLILSHIRHMSNKGMEHLYSMKYKNVVPLYDLLLEMLDAHR
LHAGGSGASRVQAFADALDDKFLHDMLAEGLRYSVIREVLPTRRA
RTFDLEVEELHTLVAEGVVVHNCAADKEVESSFAHAQVSITNEIY
QYLGEPETFLFCPTEYCGTFCYPNVSQSPYLRTVGEKLLPGIEVL
WTGPKVVSKEIPVESIEEVSKIIKRAPVIWDNIHANDYDQKRLFL
GPYKGRSTELIPRLKGVLTNPNCEFEANYVAIHTLATWYKSNMNG
VRKDVVMTDSEDSTVSIQIKLENEGSDEDIETDVLYSPQMALKLA
LTEWLQEFGVPHQYSSRGSGSGSGSGSGSGSGEKPLYTAEPVTLE
DLQLLADLFYLPYEHGPKGAQMLREFQWLRANSSVVSVNCKGKDS
EKIEEWRSRAAKFEEMCGLVMGMFTRLSNCANRTILYDMYSYVWD
IKSIMSMVKSFVQWLGCRSHSSAQFLIGDQEPWAFRGGLAGEFQR
LLPIDGANDLFFQPP*
myc-OGA(1-180)-C-intein-OGA(182, GS-544)-NLS
(SEQ ID NO: 110)
MEQKLISEEDLAIAMVQKESQATLEERESELSSNPAASAGASLEP
PAAPAPGEDNPAGAGGAAVAGAAGGARRFLCGVVEGFYGRPWVME
QRKELFRRLQKWELNTYLYAPKDDYKHRMFWREMYSVEEAEQLMT
LISAAREYEIEFIYAISPGLDITFSNPKEVSTLKRKLDQVSQFGC
RSFALLFDDIDHNMCLAEGTRIFDPVTGTTHRIEDVVDGRKPIHV
VAAAKDGTLLARPVVSWFDQGTRDVIGLRIAGGAIVWATPDHKVL
TEYGWRAAGELRKGDRVAGPGGSGNSLALSLTADQMVSALLDAEP
PILYSEYDPTSPFSEASMMGLLTNLADRELVHMINWAKRVPGFVD
LTLHDQAHLLERAWLEILMIGLVWRSMEHPGKLLFAPNLLLDRN
QGKCVEGMVEIFDMLLATSSRFRMMNLQGEEFVCLKSIILLNSGV
YTFLSSTLKSLEEKDHIHRALDKITDTLIHLMAKAGLTLQQQHQR
LAQLLLILSHIRHMSNKGMEHLYSMKYKNVVPLYDLLLEMLDAHR
LHAGGSGASRVQAFADALDDKFLHDMLAEGLRYSVIREVLPTRRA
RTFDLEVEELHTLVAEGVVVHNCAADKEVESSFAHAQVSITNEIY
QYLGEPETFLFCPTEYCGTFCYPNVSQSPYLRTVGEKLLPGIEVL
WTGPKVVSKEIPVESIEEVSKIIKRAPVIWDNIHANDYDQKRLFL
GPYKGRSTELIPRLKGVLTNPNCEFEANYVAIHTLATWYKSNMNG
VRKDVVMTDSEDSTVSIQIKLENEGSDEDIETDVLYSPQMALKLA
LTEWLQEFGVPHQYSSRGSGSGSGSGSGSGSGEKPLYTAEPVTLE
DLQLLADLFYLPYEHGPKGAQMLREFQWLRANSSVVSVNCKGKDS
EKIEEWRSRAAKFEEMCGLVMGMFTRLSNCANRTILYDMYSYVWD
IKSIMSMVKSFVQWLGCRSHSSAQFLIGDQEPWAFRGGLAGEFQR
LLPIDGANDLFFQPPKRPAATKKAGQAKKKK*
myc-OGA(1-180)-C-intein-OGA(182, GS-544)-NES
(SEQ ID NO: 111)
MEQKLISEEDLAIAMVQKESQATLEERESELSSNPAASAGASLEP
PAAPAPGEDNPAGAGGAAVAGAAGGARRFLCGVVEGFYGRPWVME
QRKELFRRLQKWELNTYLYAPKDDYKHRMFWREMYSVEEAEQLMT
LISAAREYEIEFIYAISPGLDITESNPKEVSTLKRKLDQVSQFGC
RSFALLFDDIDHNMCLAEGTRIFDPVTGTTHRIEDVVDGRKPIHV
VAAAKDGTLLARPVVSWFDQGTRDVIGLRIAGGAIVWATPDHKVL
TEYGWRAAGELRKGDRVAGPGGSGNSLALSLTADQMVSALLDAEP
PILYSEYDPTSPFSEASMMGLLTNLADRELVHMINWAKRVPGFVD
LTLHDQAHLLERAWLEILMIGLVWRSMEHPGKLLFAPNLLLDRNQ
GKCVEGMVEIFDMLLATSSRFRMMNLQGEEFVCLKSIILLNSGVY
TFLSSTLKSLEEKDHIHRALDKITDTLIHLMAKAGLTLQQQHQRL
AQLLLILSHIRHMSNKGMEHLYSMKYKNVVPLYDLLLEMLDAHRL
HAGGSGASRVQAFADALDDKFLHDMLAEGLRYSVIREVLPTRRAR
TFDLEVEELHTLVAEGVVVHNCAADKEVESSFAHAQVSITNEIYQ
YLGEPETFLFCPTEYCGTFCYPNVSQSPYLRTVGEKLLPGIEVLW
TGPKVVSKEIPVESIEEVSKIIKRAPVIWDNIHANDYDQKRLFLG
PYKGRSTELIPRLKGVLTNPNCEFEANYVAIHTLATWYKSNMNGV
RKDVVMTDSEDSTVSIQIKLENEGSDEDIETDVLYSPQMALKLAL
TEWLQEFGVPHQYSSRGSGSGSGSGSGSGSGEKPLYTAEPVTLED
LQLLADLFYLPYEHGPKGAQMLREFQWLRANSSVVSVNCKGKDSE
KIEEWRSRAAKFEEMCGLVMGMFTRLSNCANRTILYDMYSYVWDI
KSIMSMVKSFVQWLGCRSHSSAQFLIGDQEPWAFRGGLAGEFQRL
LPIDGANDLFFQPPLALKLAGLDIN*

TABLE 1
Sequences

| SEQ | | |
|---|---|---|
| ID | Descrip- | |
| NO: | tion | Sequence |
| 112 | ORF_myc- | MASMQKLISEEDLLMAMEARIRSTV |
| | hOGA | QKESQATLEERESELSSNPAASAGA |
| | | SLEPPAAPAPGEDNPAGAGGAAVAG |
| | | AAGGARRFLCGVVEGFYGRPWVMEQ |
| | | RKELFRRLQKWELNTYLYAPKDDYK |
| | | HRMFWREMYSVEEAEQLMTLISAAR |
| | | EYEIEFIYAISPGLDITFSNPKEVS |
| | | TLKRKLDQVSQFGCRSFALLFDDID |
| | | HNMCAADKEVFSSFAHAQVSITNEI |
| | | YQYLGEPETFLFCPTEYCGTFCYPN |
| | | VSQSPYLRTVGEKLLPGIEVLWTGP |
| | | KVVSKEIPVESIEEVSKIIKRAPVI |
| | | WDNIHANDYDQKRLFLGPYKGRSTE |
| | | LIPRLKGVLTNPNCEFEANYVAIHT |
| | | LATWYKSNMNGVRKDVVMTDSEDST |
| | | VSIQIKLENEGSDEDIETDVLYSPQ |
| | | MALKLALTEWLQEFGVPHQYSSRQV |
| | | AHSGAKASVVDGTPLVAAPSLNATT |
| | | VVTTVYQEPIMSQGAALSGEPTTLT |
| | | KEEEKKQPDEEPMDMVVEKQEETDH |
| | | KNDNQILSEIVEAKMAEELKPMDTD |
| | | KESIAESKSPEMSMQEDCISDIAPM |
| | | QTDEQTNKEQFVPGPNEKPLYTAEP |
| | | VTLEDLQLLADLFYLPYEHGPKGAQ |
| | | MLREFQWLRANSSVVSVNCKGKDSE |
| | | KIEEWRSRAAKFEEMCGLVMGMFTR |
| | | LSNCANRTILYDMYSYVWDIKSIMS |
| | | MVKSFVQWLGCRSHSSAQFLIGDQE |
| | | PWAFRGGLAGEFQRLLPIDGANDLF |
| | | FQPPPLTPTSKVYTIRPYFPKDEAS |
| | | VYKICREMYDDGVGLPFQSQPDLIG |
| | | DKLVGGLLSLSLDYCFVLEDEDGIC |
| | | GYALGTVDVTPFIKKCKISWIPFMQ |
| | | EKYTKPNGDKELSEAEKIMLSFHEE |
| | | QEVLPETFLANFPSLIKMDIHKKVT |
| | | DPSVAKSMMACLLSSLKANGSRGAF |
| | | CEVRPDDKRILEFYSKLGCFEIAKM |
| | | EGFPKDVVILGRSL* |
| 113 | ORF_myc- | ATGGCATCAATGCAGAAGCTGATCT |
| | hOGA | CAGAGGAGGACCTGCTTATGGCCAT |
| | | GGAGGCCCGAATTCGGTCGACCGTG |
| | | CAGAAGGAGAGTCAAGCGACGTTGG |
| | | AGGAGCGGGAGAGCGAGCTCAGCTC |
| | | CAACCCTGCCGCCTCTGCGGGGGCA |
| | | TCGCTGGAGCCGCCGGCAGCTCCGG |
| | | CACCCGGAGAAGACAACCCCGCCGG |
| | | GGCTGGGGGAGCGGCGGTGGCCGGG |
| | | GCTGCAGGAGGGGCTCGGCGGTTCC |
| | | TCTGCGGTGTGGTGGAAGGATTTTA |
| | | TGGAAGACCTTGGGTTATGGAACAG |
| | | AGAAAAGAACTCTTTAGAAGGCTCC |
| | | AGAAATGGGAATTAAATACATACTT |
| | | GTATGCCCCAAAAGATGACTACAAA |
| | | CATAGGATGTTTTGGCGAGAGATGT |
| | | ATTCAGTGGAGGAAGCTGAGCAACT |
| | | TATGACTCTCATCTCTGCTGCACGA |
| | | GAATATGAGATAGAGTTCATCTATG |
| | | CGATCTCACCTGGATTGGATATCAC |
| | | TTTTTCTAACCCCAAGGAAGTATCC |
| | | ACATTGAAACGTAAATTGGACCAGG |
| | | TTTCTCAGTTTGGGTGCAGATCATT |
| | | TGCTTTGCTTTTTGATGATATAGAC |
| | | CATAATATGTGTGCAGCAGACAAAG |
| | | AGGTATTCAGTTCTTTTGCTCATGC |
| | | CCAAGTCTCCATCACAAATGAAATC |
| | | TATCAGTACCTAGGAGAGCCAGAAA |
| | | CTTTCCTCTTCTGTCCCACAGAATA |
| | | CTGTGGCACTTTCTGTTATCCAAAT |
| | | GTGTCTCAGTCTCCATATTTAAGGA |
| | | CTGTGGGTGAAAAGCTTCTACCTGG |
| | | AATTGAAGTGCTTTGGACAGGTCCC |
| | | AAAGTTGTTTCTAAAGAAATTCCAG |
| | | TAGAGTCCATCGAAGAGGTTTCTAA |
| | | GATTATTAAGAGAGCTCCAGTAATC |
| | | TGGGATAACATTCATGCTAATGATT |
| | | ATGATCAGAAGAGACTGTTTCTGGG |
| | | CCCGTACAAAGGAAGATCCACAGAA |
| | | CTCATCCCACGGTTAAAAGGAGTCC |
| | | TCACTAATCCAAATTGTGAATTTGA |
| | | AGCCAACTACGTTGCTATCCACACC |
| | | CTTGCCACCTGGTACAAATCAAACA |
| | | TGAATGGAGTGAGAAAAGATGTAGT |
| | | GATGACTGACAGTGAAGATAGTACT |
| | | GTGTCCATCCAGATAAAATTAGAAA |
| | | ATGAAGGCAGTGATGAAGATATTGA |
| | | AACTGATGTACTCTATAGTCCACAG |
| | | ATGGCTCTAAAGCTAGCATTAACAG |
| | | AATGGTTGCAAGAGTTTGGTGTGCC |
| | | TCATCAATACAGCAGTAGGCAAGTT |
| | | GCACACAGTGGAGCTAAAGCAAGTG |
| | | TAGTTGATGGGACTCCTTTAGTTGC |
| | | AGCACCCTCTTTAAATGCCACAACC |
| | | GTAGTAACAACAGTTTATCAGGAGC |
| | | CCATTATGAGCCAGGGAGCAGCCTT |
| | | GAGTGGTGAGCCTACTACTCTGACC |
| | | AAGGAAGAAGAAAAGAAACAGCCTG |
| | | ATGAAGAACCCATGGACATGGTGGT |
| | | GGAAAAACAAGAAGAAACGGACCAC |
| | | AAGAATGACAATCAAATACTGAGTG |
| | | AAATTGTTGAAGCGAAAATGGCAGA |
| | | GGAATTGAAACCAATGGACACTGAT |
| | | AAAGAGAGCATAGCTGAATCAAAAT |
| | | CCCCAGAGATGTCCATGCAAGAAGA |
| | | TTGTATTAGTGACATTGCCCCCATG |
| | | CAAACTGATGAACAGACAAACAAGG |
| | | AGCAGTTTGTGCCAGGTCCAAATGA |
| | | AAAGCCTTTGTACACTGCGGAACCA |
| | | GTGACCCTGGAGGATTTGCAGTTAC |
| | | TTGCTGATCTATTCTACCTTCCTTA |
| | | CGAGCATGGACCCAAAGGAGCACAG |
| | | ATGTTACGGGAATTTCAATGGCTTC |
| | | GAGCAAATAGTAGTGTTGTCAGTGT |
| | | CAATTGCAAAGGAAAAGACTCTGAA |
| | | AAAATTGAAGAATGGCGGTCACGAG |
| | | CAGCCAAGTTTGAAGAGATGTGTGG |
| | | ACTAGTGATGGGAATGTTCACTCGG |
| | | CTCTCCAATTGTGCCAACAGGACAA |
| | | TTCTTTATGACATGTACTCCTATGT |
| | | TTGGGATATCAAGAGTATAATGTCT |
| | | ATGGTGAAGTCTTTTGTACAGTGGT |
| | | TAGGGTGTCGTAGTCATTCTTCAGC |
| | | ACAATTCTTAATTGGAGACCAAGAA |
| | | CCCTGGGCCTTTAGAGGTGGTCTAG |
| | | CAGGAGAGTTCCAGCGTTTGCTGCC |
| | | AATTGATGGGGCAAATGATCTCTTT |
| | | TTTCAGCCACCTCCACTGACTCCTA |
| | | CCTCCAAAGTTTATACTATCAGACC |
| | | TTATTTTCCTAAGGATGAGGCATCC |
| | | GTGTACAAGATTTGCAGAGAAATGT |
| | | ATGACGATGGAGTGGGTTTACCCTT |
| | | TCAAAGTCAGCCTGATCTTATTGGA |
| | | GACAAGTTAGTAGGAGGGCTGCTTT |
| | | CCCTCAGCCTGGATTACTGCTTTGT |
| | | CCTAGAAGATGAAGATGGCATATGT |
| | | GGTTATGCCTTGGGCACTGTAGATG |
| | | TGACCCCCTTTATTAAAAAATGTAA |
| | | AATTTCCTGGATCCCCTTCATGCAG |
| | | GAGAAGTATACCAAGCCAAATGGTG |
| | | ACAAGGAACTCTCTGAGGCTGAGAA |
| | | AATAATGTTGAGTTTCCATGAAGAA |
| | | CAGGAAGTACTGCCAGAAACTTTCC |
| | | TTGCTAATTTCCCTTCTCTGATAAA |
| | | GATGGACATTCACAAAAAAGTAACT |
| | | GACCCAAGTGTGGCCAAAAGCATGA |
| | | TGGCTTGCCTCCTGTCTTCACTGAA |
| | | GGCTAATGGCTCCCGGGGAGCTTTC |
| | | TGTGAAGTGAGACCAGATGATAAAA |
| | | GAATTCTGGAATTTTACAGCAAGTT |
| | | AGGATGTTTTGAAATTGCAAAAATG |
| | | GAAGGATTTCCAAAGGATGTGGTTA |
| | | TACTTGGTCGGAGCCTGTAA |
| 114 | ORF_myc- | ATGGAGCAGAAGCTGATCAGCGAGG |
| | OGA | AGGACCTGGCGATCGCAATGGTGCA |
| | (1-400) | GAAGGAGAGTCAAGCGACGTTGGAG |
| | | GAGCGGGAGAGCGAGCTCAGCTCCA |
| | | ACCCTGCCGCCTCTGCGGGGGCATC |
| | | GCTGGAGCCGCCGGCAGCTCCGGCA |
| | | CCCGGAGAAGACAACCCCGCCGGGG |
| | | CTGGGGGAGCGGCGGTGGCCGGGGC |
| | | TGCAGGAGGGGCTCGGCGGTTCCTC |
| | | TGCGGTGTGGTGGAAGGATTTTATG |
| | | GAAGACCTTGGGTTATGGAACAGAG |
| | | AAAAGAACTCTTTAGAAGGCTCCAG |
| | | AAATGGGAATTAAATACATACTTGT |
| | | ATGCCCCAAAAGATGACTACAAACA |
| | | TAGGATGTTTTGGCGAGAGATGTAT |
| | | TCAGTGGAGGAAGCTGAGCAACTTA |
| | | TGACTCTCATCTCTGCTGCACGAGA |
| | | ATATGAGATAGAGTTCATCTATGCG |
| | | ATCTCACCTGGATTGGATATCACTT |
| | | TTTCTAACCCCAAGGAAGTATCCAC |
| | | ATTGAAACGTAAATTGGACCAGGTT |
| | | TCTCAGTTTGGGTGCAGATCATTTG |
| | | CTTTGCTTTTTGATGATATAGACCA |
| | | TAATATGTGTGCAGCAGACAAAGAG |
| | | GTATTCAGTTCTTTTGCTCATGCCC |
| | | AAGTCTCCATCACAAATGAAATCTA |
| | | TCAGTACCTAGGAGAGCCAGAAACT |
| | | TTCCTCTTCTGTCCCACAGAATACT |
| | | GTGGCACTTTCTGTTATCCAAATGT |
| | | GTCTCAGTCTCCATATTTAAGGACT |
| | | GTGGGTGAAAAGCTTCTACCTGGAA |
| | | TTGAAGTGCTTTGGACAGGTCCCAA |
| | | AGTTGTTTCTAAAGAAATTCCAGTA |
| | | GAGTCCATCGAAGAGGTTTCTAAGA |
| | | TTATTAAGAGAGCTCCAGTAATCTG |
| | | GGATAACATTCATGCTAATGATTAT |
| | | GATCAGAAGAGACTGTTTCTGGGCC |
| | | CGTACAAAGGAAGATCCACAGAACT |
| | | CATCCCACGGTTAAAAGGAGTCCTC |
| | | ACTAATCCAAATTGTGAATTTGAAG |
| | | CCAACTACGTTGCTATCCACACCCT |
| | | TGCCACCTGGTACAAATCAAACATG |
| | | AATGGAGTGAGAAAAGATGTAGTGA |
| | | TGACTGACAGTGAAGATAGTACTGT |
| | | GTCCATCCAGATAAAATTAGAAAAT |
| | | GAAGGCAGTGATGAAGATATTGAAA |
| | | CTGATGTACTCTATAGTCCACAGAT |
| | | GGCTCTAAAGCTAGCATTAACAGAA |
| | | TGGTTGCAAGAGTTTGGTGTGCCTC |
| | | ATCAATACAGCAGTAGGTAA |
| 115 | ORF_myc- | MEQKLISEEDLAIAMVQKESQATLE |
| | OGA | ERESELSSNPAASAGASLEPPAAPA |
| | (1-400) | PGEDNPAGAGGAAVAGAAGGARRFL |
| | ORF_myc- | CGVVEGFYGRPWVMEQRKELFRRLQ |
| | | KWELNTYLYAPKDDYKHRMFWREMY |
| | | SVEEAEQLMTLISAAREYEIEFIYA |
| | | ISPGLDITFSNPKEVSTLKRKLDQV |
| | | SQFGCRSFALLFDDIDHNMCAADKE |
| | | VFSSFAHAQVSITNEIYQYLGEPET |
| | | FLFCPTEYCGTFCYPNVSQSPYLRT |
| | | VGEKLLPGIEVLWTGPKVVSKEIPV |
| | | ESIEEVSKIIKRAPVIWDNIHANDY |
| | | DQKRLFLGPYKGRSTELIPRLKGVL |
| | | TNPNCEFEANYVAIHTLATWYKSNM |
| | | NGVRKDVVMTDSEDSTVSIQIKLEN |
| | | EGSDEDIETDVLYSPQMALKLALTE |
| | | WLQEFGVPHQYSSR* |
| 116 | OGA | ATGGAGCAGAAGCTGATCAGCGAGG |
| | (1-400) | AGGACCTGGCGATCGCAATGGTGCA |
| | D174N | GAAGGAGAGTCAAGCGACGTTGGAG |
| | | GAGCGGGAGAGCGAGCTCAGCTCCA |
| | | ACCCTGCCGCCTCTGCGGGGGCATC |
| | | GCTGGAGCCGCCGGCAGCTCCGGCA |
| | | CCCGGAGAAGACAACCCCGCCGGGG |
| | | CTGGGGGAGCGGCGGTGGCCGGGGC |
| | | TGCAGGAGGGGCTCGGCGGTTCCTC |
| | | TGCGGTGTGGTGGAAGGATTTTATG |
| | | GAAGACCTTGGGTTATGGAACAGAG |
| | | AAAAGAACTCTTTAGAAGGCTCCAG |
| | | AAATGGGAATTAAATACATACTTGT |
| | | ATGCCCCAAAAGATGACTACAAACA |
| | | TAGGATGTTTTGGCGAGAGATGTAT |
| | | TCAGTGGAGGAAGCTGAGCAACTTA |
| | | TGACTCTCATCTCTGCTGCACGAGA |
| | | ATATGAGATAGAGTTCATCTATGCG |
| | | ATCTCACCTGGATTGGATATCACTT |
| | | TTTCTAACCCCAAGGAAGTATCCAC |
| | | ATTGAAACGTAAATTGGACCAGGTT |
| | | TCTCAGTTTGGGTGCAGATCATTTG |
| | | CTTTGCTTTTTAATGATATAGACCA |
| | | TAATATGTGTGCAGCAGACAAAGAG |
| | | GTATTCAGTTCTTTTGCTCATGCCC |
| | | AAGTCTCCATCACAAATGAAATCTA |
| | | TCAGTACCTAGGAGAGCCAGAAACT |
| | | TTCCTCTTCTGTCCCACAGAATACT |
| | | GTGGCACTTTCTGTTATCCAAATGT |
| | | GTCTCAGTCTCCATATTTAAGGACT |
| | | GTGGGTGAAAAGCTTCTACCTGGAA |
| | | TTGAAGTGCTTTGGACAGGTCCCAA |
| | | AGTTGTTTCTAAAGAAATTCCAGTA |
| | | GAGTCCATCGAAGAGGTTTCTAAGA |
| | | TTATTAAGAGAGCTCCAGTAATCTG |
| | | GGATAACATTCATGCTAATGATTAT |
| | | GATCAGAAGAGACTGTTTCTGGGCC |
| | | CGTACAAAGGAAGATCCACAGAACT |
| | | CATCCCACGGTTAAAAGGAGTCCTC |
| | | ACTAATCCAAATTGTGAATTTGAAG |
| | | CCAACTACGTTGCTATCCACACCCT |
| | | TGCCACCTGGTACAAATCAAACATG |
| | | AATGGAGTGAGAAAAGATGTAGTGA |
| | | TGACTGACAGTGAAGATAGTACTGT |
| | | GTCCATCCAGATAAAATTAGAAAAT |
| | | GAAGGCAGTGATGAAGATATTGAAA |
| | | CTGATGTACTCTATAGTCCACAGAT |
| | | GGCTCTAAAGCTAGCATTAACAGAA |
| | | TGGTTGCAAGAGTTTGGTGTGCCTC |
| | | ATCAATACAGCAGTAGGTAA |
| 117 | ORF_myc- | MEQKLISEEDLAIAMVQKESQATLE |
| | (1-400) | ERESELSSNPAASAGASLEPPAAPA |
| | D174N | PGEDNPAGAGGAAVAGAAGGARRFL |
| | | CGVVEGFYGRPWVMEQRKELFRRLQ |
| | | KWELNTYLYAPKDDYKHRMFWREMY |
| | | SVEEAEQLMTLISAAREYEIEFIYA |
| | | ISPGLDITESNPKEVSTLKRKLDQV |
| | | SQFGCRSFALLFNDIDHNMCAADKE |
| | | VFSSFAHAQVSITNEIYQYLGEPET |
| | | FLFCPTEYCGTFCYPNVSQSPYLRT |
| | | VGEKLLPGIEVLWTGPKVVSKEIPV |
| | | ESIEEVSKIIKRAPVIWDNIHANDY |
| | | DQKRLFLGPYKGRSTELIPRLKGVL |
| | | TNPNCEFEANYVAIHTLATWYKSNM |
| | | NGVRKDVVMTDSEDSTVSIQIKLEN |
| | | EGSDEDIETDVLYSPQMALKLALTE |
| | | WLQEFGVPHQYSSR* |
| 118 | ORF_HA- | ATGGCATACCCATACGATGTTCCAG |
| | OGA | ATTACGCTGCGATCGCAGAAAAGCC |
| | (544-706) | TTTGTACACTGCGGAACCAGTGACC |
| | | CTGGAGGATTTGCAGTTACTTGCTG |
| | | ATCTATTCTACCTTCCTTACGAGCA |
| | | TGGACCCAAAGGAGCACAGATGTTA |
| | | CGGGAATTTCAATGGCTTCGAGCAA |
| | | ATAGTAGTGTTGTCAGTGTCAATTG |
| | | CAAAGGAAAAGACTCTGAAAAAATT |
| | | GAAGAATGGCGGTCACGAGCAGCCA |
| | | AGTTTGAAGAGATGTGTGGACTAGT |
| | | GATGGGAATGTTCACTCGGCTCTCC |
| | | AATTGTGCCAACAGGACAATTCTTT |
| | | ATGACATGTACTCCTATGTTTGGGA |
| | | TATCAAGAGTATAATGTCTATGGTG |
| | | AAGTCTTTTGTACAGTGGTTAGGGT |
| | | GTCGTAGTCATTCTTCAGCACAATT |
| | | CTTAATTGGAGACCAAGAACCCTGG |
| | | GCCTTTAGAGGTGGTCTAGCAGGAG |
| | | AGTTCCAGCGTTTGCTGCCAATTGA |
| | | TGGGGCAAATGATCTCTTTTTTCAG |
| | | CCACCTTAA |
| 119 | ORF_HA- | MAYPYDVPDYAAIAEKPLYTAEPVT |
| | OGA | LEDLQLLADLFYLPYEHGPKGAQML |
| | (544-706) | REFQWLRANSSVVSVNCKGKDSEKI |
| | | EEWRSRAAKFEEMCGLVMGMFTRLS |
| | | NCANRTILYDMYSYVWDIKSIMSMV |
| | | KSFVQWLGCRSHSSAQFLIGDQEPW |
| | | AFRGGLAGEFQRLLPIDGANDLFFQ |
| | | PP* |
| 120 | ORF_HA- | ATGGCATACCCATACGATGTTCCAG |
| | nGFP- | ATTACGCTGCGATCGCACAGGTGCA |
| | (EAAAK)4- | GCTGGTGGAGTCTGGAGGAGCTCTG |
| | OGA | GTGCAGCCTGGAGGAAGCCTGCGCC |
| | (544-706) | TGAGCTGTGCAGCTAGCGGATTTCC |
| | | TGTGAACCGCTACAGCATGCGCTGG |
| | | TACCGCCAGGCTCCTGGTAAAGAGC |
| | | GCGAGTGGGTGGCTGGAATGAGCAG |
| | | CGCTGGAGATCGCAGCAGCTACGAG |
| | | GACAGCGTGAAAGGACGCTTTACAA |
| | | TCAGCCGCGATGATGCTCGCAACAC |
| | | AGTGTACCTGCAGATGAACTCTCTG |
| | | AAACCTGAGGACACTGCTGTGTACT |
| | | ACTGTAACGTGAACGTGGGTTTCGA |
| | | GTACTGGGGACAGGGAACACAGGTG |
| | | ACAGTGAGCTCTGGCGCGCCAGAGG |
| | | CAGCTGCAAAGGAGGCAGCTGCAAA |
| | | GGAGGCAGCTGCAAAGGAGGCAGCT |
| | | GCAAAGTTAATTAAGGAAAAGCCTT |
| | | TGTACACTGCGGAACCAGTGACCCT |
| | | GGAGGATTTGCAGTTACTTGCTGAT |
| | | CTATTCTACCTTCCTTACGAGCATG |
| | | GACCCAAAGGAGCACAGATGTTACG |
| | | GGAATTTCAATGGCTTCGAGCAAAT |
| | | AGTAGTGTTGTCAGTGTCAATTGCA |
| | | AAGGAAAAGACTCTAAAAAAATTGA |
| | | AGAATGGCGGTCACGAGCAGCCAAG |
| | | TTTGAAGAGATGTGTGGACTAGTGA |
| | | TGGGAATGTTCACTCGGCTCTCCAA |
| | | TTGTGCCAACAGGACAATTCTTTAT |
| | | GACATGTACTCCTATGTTTGGGATA |
| | | TCAAGAGTATAATGTCTATGGTGAA |
| | | GTCTTTTGTACAGTGGTTAGGGTGT |
| | | CGTAGTCATTCTTCAGCACAATTCT |
| | | TAATTGGAGACCAAGAACCCTGGGC |
| | | CTTTAGAGGTGGTCTAGCAGGAGAG |
| | | TTCCAGCGTTTGCTGCCAATTGATG |
| | | GGGCAAATGATCTCTTTTTTCAGCC |
| | | ACCTTAA |
| 121 | ORF_HA- | MAYPYDVPDYAAIAQVQLVESGGAL |
| | nGFP- | VQPGGSLRLSCAASGFPVNRYSMRW |
| | (EAAAK)4- | YRQAPGKEREWVAGMSSAGDRSSYE |
| | OGA | DSVKGRFTISRDDARNTVYLQMNSL |
| | (544-706) | KPEDTAVYYCNVNVGFEYWGQGTQV |
| | | TVSSGAPEAAAKEAAAKEAAAKEAA |
| | | AKLIKEKPLYTAEPVTLEDLQLLAD |
| | | LFYLPYEHGPKGAQMLREFQWLRAN |
| | | SSVVSVNCKGKDSKKIEEWRSRAAK |
| | | FEEMCGLVMGMFTRLSNCANRTILY |
| | | DMYSYVWDIKSIMSMVKSFVQWLGC |
| | | RSHSSAQFLIGDQEPWAFRGGLAGE |
| | | FQRLLPIDGANDLFFQPP* |

TABLE 2
List of plasmids

| No. | Plasmid name | Abbreviated Names | Details |
|---|---|---|---|
| 1 | pCMV-myc-OGA | fl-OGA | Full length OGA with a |
| | | | myc tag |
| 2 | pcDNA3.1-myc-OGA(cat) | myc-OGA(cat)/N3 | |
| 3 | pcDNA3.1-myc-OGA(ΔHAT) | | |
| 4 | pcDNA3.1-myc-OGA(GS-ΔHAT) | | |
| 5 | pcDNA3.1-HA-OGA | | Full length OGA with a |
| | | | HA tag |
| 6 | pcDNA3.1-HA-nGFP-(EAAAK)4- | | A linker with four |
| | OGA | | (EAAAK (SEQ ID |
| 7 | pcDNA3.1-HA-nGFP-(EAAAK)4- | | NO: 122)) repeats is |
| | OGA(cat) | | used between nGFP |
| 8 | pcDNA3.1-HA-nGFP-(EAAAK)4- | | and indicated OGAs. |
| | OGA(ΔHAT) | | |
| 9 | pcDNA3.1-HA-nGFP-(EAAAK)4- | | |
| | OGA(GS-ΔHAT) | | |
| 10 | pcDNA3.1-myc-OGA(1-413) | N1 | myc-tag for N |
| 11 | pcDNA3.1-myc-OGA(1-400) | N2 | fragments and HA-tag |
| 12 | pcDNA3.1-myc-OGA(1-400)D174N | N2(D174N) | for C fragments |
| 13 | pcDNA3.1-HA-OGA(414-916) | C1 | |
| 14 | pcDNA3.1-HA-OGA(414-706) | C2 | |
| 15 | pcDNA3.1-HA-OGA(544-706) | C3 | |
| 16 | pcDNA3.1-HA-OGA(554-706) | C4 | |
| 17 | pcDNA3.1-myc-nGFP-OGA(cat) | nGFP-OGA(cat)/N3 | A linker with four |
| | | | (EAAAK (SEQ ID |
| 18 | pcDNA3.1-myc-nGFP-(EAAAK)4- | nGFP-N2 | NO: 122)) repeats is |
| | OGA(1-400) | | used between nGFP |
| 19 | pcDNA3.1-myc-nGFP-(EAAAK)4- | nGFP-N2(D174N) | and indicated OGAs; |
| | OGA(1-400)D174N | | myc-tag for N |
| 20 | pcDNA3.1-myc-nGFP-(EAAAK)4- | nGFP-OGA(GS- | fragments and HA-tag |
| | OGA(GS-ΔHAT) | ΔHAT) | for C fragments |
| 21 | pcDNA3.1-HA-nGFP-(EAAAK)4- | nGFP-C3 | |
| | OGA(544-706) | | |
| 22 | pcDNA3.1-HA-nGFP-(EAAAK)4- | nGFP-C4 | |
| | OGA(554-706) | | |
| 23 | pcDNA3.1-Nup62-GFP-Flag | Nup62-GFP-Flag | |
| 24 | pcDNA3.1-GFP-Flag-Nup62-EPEA | GFP-Nup62 | GFP and Flag tag at N |
| 25 | pcDNA3.1-GFP-Flag-Sp1-EPEA | GFP-Sp1 | terminus; EPEA tag at |
| 26 | pcDNA3.1-GFP-Flag-JunB-EPEA | GFP-JunB | C terminus |
| 27 | pcDNA3.1-GFP-Flag-c-Jun-EPEA | GFP-c-Jun | |
| 28 | pcDNA3.1-HA-nUbc-(EAAAK)4- | nUbc-C3 | nUbc-fused C3 |
| | OGA(544-706) | | fragment |
| 29 | pcDNA3.1-HA-nEPEA-(EAAAK)4- | nEPEA-C3 | nEPEA-fused C3 |
| | OGA(544-706) | | fragment |
| 30 | pcDNA3.1-HA-nBC2-(EAAAK)4- | nBC2-C3 | nBC2-fused C3 |
| | OGA(544-706) | | fragment |
| 31 | pcDNA3.1-Nup62-Ubc-Flag-EPEA | Nup62-Ubc-EPEA | Ubc, Flag and EPEA |
| 32 | pcDNA3.1-c-Fos-Ubc-Flag-EPEA | c-Fos-Ubc | tag at C terminus |
| 33 | pcDNA3.1-BC2-Nup62-Flag-EPEA | BC2-Nup62-EPEA | BC2 tag at N terminus; |
| | | | Flag and EPEA at C |
| | | | terminus |
| 34 | 3xAP1pGL3 | | AP-1 responsive |
| | | | luciferase reporter |
| 35 | pCMV3-OGT-His | OGT-His | His tag at C terminus |

Generation of MCF-7 stable cell lines. Replication-deficient lentivirus was produced by transient transfection of 0.75 μg psPAX2 (Addgene #12260), 0.25 μg pMD2.G (Addgene #12259), and 1 μg OGA-intein(C181) into HEK293T cells seeded in a 6-well plate. Viral supernatants were collected after 48 hours and passed through a 0.45 μm filter. Dilutions of the filtered supernatant into fresh medium containing 10 μg/mL polybrene were added to infect MCF-7 cells. 48 hours post infection, cells were collected and resuspended with fresh medium. EGFP-positive cells were sorted using CytoFLEX SRT (Beckman Coulter, USA).

Antibodies and reagents. Antibodies including anti-His (12698), anti-Myc (2276), anti-HA (3724), anti-GAPDH (5174), and anti-OGT (24083) were purchased from Cell Signaling Technology. Anti-O-GlcNAc (RL2) (ab2739) antibody was purchased from Abcam. Horseradish peroxidase (HRP)-conjugated secondary antibodies were purchased from Rockland Immunochemicals. IRDye secondary antibodies were purchased from LI-COR Biosciences. AlexaFluor 488 anti-rabbit IgG (A11008), AlexaFluor 568 anti-mouse IgG (A11004), and NucBlue Fixed Cell Stain ReadyProbes reagent (R37606) were purchased from Invitrogen. Antibody-conjugated beads for immunoprecipitation were anti-EPEA CaptureSelect™ C-tag affinity matrix (Thermo Scientific, 191307005) and His-Tag Dynabeads (Invitrogen, 10103D). Thiamet-G (S7213) and 4-hydroxytamoxifen (Afimoxifene, S7827) were purchased from Selleckchem.

Immunoprecipitation and Immunoblot assays. Cells with indicated treatments were harvested and washed with PBS once, then lysed with M-PER lysis buffer (Thermo Scientific, 78501) containing 1× protease inhibitor cocktail and 10 μM Thiamet-G. Equal amounts of protein determined by the BCA assay were diluted with PBS and incubated with prewashed C-tag affinity matrix for 1 h or His-Tag Dynabeads for 20 min at room temperature, respectively, according to the manufacturer's instructions. Following three times rinses with PBS, the enriched proteins were eluted with the SDS sample buffer and subjected to SDS-PAGE.

For immunoprecipitation of proteins with the EPEA tag at the C-terminus, cell lysates with equal amounts of protein were diluted with PBS and incubated with C-tag affinity matrix for 1 hour at room temperature, with end-to-end rotation. After washing three times with PBS buffer, the enriched proteins were eluted with SDS sample buffer and subjected to SDS-PAGE.

For immunoprecipitation of proteins with a His tag, cells were lysed in a buffer containing 50 mM Tris HCl (pH 8.0), 150 mM NaCl, 1% Triton X-100, 5% glycerol, 1× protease inhibitor cocktail, and 10 μM Thiamet-G. Cell lysates with equal amounts of protein were diluted with wash buffer (50 mM Tris HCl (pH 8.0), 150 mM NaCl, 0.01% Tween-20) and incubated with prewashed His-Tag Dynabeads at room temperature for 20 minutes with mixing following the manufacturer's instructions. After washing four times with wash buffer, the enriched proteins were eluted with SDS sample buffer and subjected to SDS-PAGE.

For immunoblotting analysis, proteins were transferred to a nitrocellulose membrane using an iBlot system (Invitrogen). Membranes were incubated with the blocking buffer (5% BSA in TBS-T), the primary antibodies (diluted 1:1,000), and the secondary antibodies (diluted 1:10,000) sequentially. Immunoblot images were captured using Azure Imager C600 and analyzed with Fiji ImageJ for converting all IR fluorescence western blot images to grayscale images.

In vitro OGA activity assay. OGA activity assay was performed as described previously (30). The reaction comprising of whole cell lysate, 50 mM sodium cacodylate, pH 6.4, 0.3% BSA, 100 mM N-acetyl-D-galactosamine (GalNAc) (MA04390, Carbosynth), and 1 mM 4-methylumbelliferyl (4MU)-GlcNAc (M2133, Sigma) or 4MU-GalNAc (M3029, TCI) were set up in black 96-well plates and incubated at 37° C. for 1.5 hours and quenched with glycine, pH 10.75 (150 mM final concentration). Fluorescence intensity was measured using a multi-mode microplate reader FilterMax F3 (Molecular Devices LLC, excitation, 368 nm; emission, 450 nm; sensitivity, 100). To exclude lysosomal hexoaminidase activity, 4MU-GalNAc fluorescence needs to be subtracted from 4MU-GlcNAc fluorescence.

Quantitative RT-PCR analysis. Total RNA of HEK293T cells under indicated treatments were extracted with RNeasy® Plus mini Kit (QIAGEN, 74134), and 1 μg RNA was subjected to reverse transcription using PrimeScript™ RT reagent Kit (Takara, RR037A), followed by quantitative PCR analysis using QuantiTect™ SYBR Green PCR Kit (QIAGEN, 204141) and Bio-Rad CFX96 Real-Time PCR detection system. The primers used in this study are listed below: Ogt (the forward primer, 5′-CAGGAAGGCTATTGCTGAGAGG-3′ (SEQ ID NO: 101) and the reverse primer, 5′-CGGAACTCACATATCCTACACGC-3′ (SEQ ID NO: 102)), Mega5 (Oga, the forward primer, 5′-GCAAGAGTTTGGTGTGCCTCATC-3′ (SEQ ID NO: 103) and the reverse primer, 5′-GTGCTGCAACTAAAGGAGTCCC-3′ (SEQ ID NO: 104)), Gapdh (the forward primer, 5′-GTCTCCTCTGACTTCAACAGCG-3′ (SEQ ID NO: 105) and the reverse primer, 5′-ACCACCCTGTTGCTGTAGCCAA-3′ (SEQ ID NO: 106)) as an internal control.

Cell viability assay. Cell viability was assessed by CCK-8 assay. MCF-7 cells were seeded in a 96-well plate at a density of 10,000 cells per well with 2% FBS and treated with 4-HT after 24 hours at the indicated concentrations. After 48-hour treatment, cells were incubated with 10% CCK-8 reagent (TargetMol, C0005) for 1-2 h at 37° C. The absorbance value of each well at 450 nm was detected using a multi-mode microplate reader Spark (TECAN, Absorbance: 450 nm, reference: 600 nm, sensitivity, 100).

Detection of cell apoptosis. Apoptosis analysis of MCF-7 stable cell lines under indicated 4-HT treatments was performed using Annexin V-mCherry Apoptosis Detection Kit (Beyotime, C1069M) according to the manufacturer's instructions. Briefly, MCF-7 cells stably expressing OGA-intein(C181) or its inactive form were seeded in a 6-well plate at a density of 10,000 cells per well and cultured with 2% FBS in the presence or absence of indicated concentration of 4-HT. After 24 hours, cells were collected and resuspended with binding buffer containing Annexin V-mCherry, which were incubated at room temperature for 20 minutes, followed by a 5-minute DAPI incubation before analysis. Fluorescence intensity from mCherry and DAPI channels were detected using Attune™ N×T Flow Cytometer (Invitrogen™). Data was analyzed by FlowJo v10.

Chemoenzymatic labeling of O-GlcNAcylated proteins. Purification of GalT1 (Y289L) enzyme and labeling of O-GlcNAcylated proteins with GalNAz were performed according to the procedure of Hsich-Wilson and co-workers (51). Briefly, cell samples in 15-cm dishes were harvested and washed by PBS once. Cells were lysed in 2% SDS/PBS by heating at 95° C. for 5 minutes, followed by sonication. Protein concentrations were determined and then subjected to reduction and alkylation using 25 mM DTT at 95° C. for 5 minutes and 50 mM iodoacetamide at room temperature for 1 hour, respectively. Proteins were precipitated using the methanol/chloroform mix (aqueous phase: CH3OH:CHCl₃=4:4:1) and resuspended in 1% SDS, 20 mM HEPES (pH 7.9) buffer with a concentration of 3.75 mg/mL. For 150 μg proteins, the reaction was set up as the following: H₂O (49 μL), 2.5× GalT labeling buffer (80 μL, final concentrations: 50 mM NaCl, 20 mM HEPES, 2% NP-40, pH 7.9), 100 mM MnCl₂ (11 μL), 500 M UDP-GalNAz (10 μL), 2 mg/mL GalT (Y289L) (10 μL). The reaction was gently rotated at 4° C. for at least 20 hours, and the proteins were precipitated as described above. The starting material for proteomics is 3 mg of proteins per treatment.

Quantitative chemical proteomics. A click chemistry was performed based on the procedure of Woo and co-workers (38). Proteins after GalT1 were resuspended in 1% SDS/PBS and incubated with 100 μM THPTA, 0.5 mM CuSO₄, 200 UM Biotin-Alkyne probe, and 2.5 mM fresh sodium ascorbate for click chemistry at 37° C. for 4 hours. After protein precipitation and resuspension, 400 μL prewashed streptavidin beads slurry was added into the diluted protein solutions for a 4 hour incubation at room temperature with gentle rotation. The beads were washed with 0.2% SDS/PBS, PBS, and H₂O sequentially and then subjected to trypsin digestion at 37° C. for 16 hours using 2 μM trypsin (Promega, V5111) in 500 μL of PBS containing 500 nM urea, 1 mM CaCl₂). The eluant was collected and desalted by C18 Tips following the manufacturer's instructions and resuspended in 20 μL of 50 mM TEAB buffer. For each sample, 5 μL of the corresponding amine-based TMT 16-plex reagents (Thermo Scientific, A44520; 11.9 μg/μL) was added and reacted for 1 hour at room temperature, which was quenched with 2 μL 5% hydroxylamine solution. The combined mixture was concentrated to dryness before further fractionation into six samples using a High pH Reversed-Phase Peptide Fractionation Kit (Pierce, 84868).

Mass spectrometry acquisition procedures. A Thermo Scientific EASY-nLC 1000 system was coupled to an Orbitrap Fusion™ Tribrid with a nano-electrospray ion source. Mobile phases A and B were water with 0.1% formic acid (vol/vol) in water and acetonitrile were used as mobile phases A and B, respectively. A liner gradient from 4% to 32% B within 50 minutes, followed by an increase to 50% B within 10 minutes and further to 98% B within 10 minutes and re-equilibration was conducted for peptide separation. The instrument parameters were chosen as previously described (1)

Mass spectrometry data analysis. The raw data was processed using Proteome Discoverer 2.4 (Thermo Fisher Scientific). The UniProt/SwissProt human (Homo sapiens) protein database (19 Aug. 2016, 20,156 total entries) and contaminant proteins and the Sequest HT algorithm were applied for searches with the following setting: spectra with a signal-to-noise ratio greater than 1.5; trypsin as enzyme, 2 missed cleavages; variable oxidation on methionine residues (15.995 Da); static carboxyamidomethylation of cysteine residues (57.021 Da), static TMT labeling (304.207 Da) at lysine residues and peptide N-termini; 10 ppm mass error tolerance on precursor ions, and 0.02 Da mass error on fragment ions. Data were filtered with a peptide-to-spectrum match of 1% FDR using Percolator. The TMT reporter ions were quantified using the Reporter Ions Quantifier without normalization. For the obtained proteome, further filters were set up: protein FDR confidence is high, unique peptides are greater than 2, master protein only and exclude all contaminant proteins. For P-value and fold change calculations, the data were further processed using a custom algorithm as described before (1). Cellular component analysis was performed with an online GO Enrichment Analysis tool powered by PANTHER (geneontology.org).

Immunofluorescence microscopy. Cells were seeded on either poly-L-lysine coated glass coverslips (Neuvitro Corporation, H-22-1.5-pll) placed in single wells of a 6-well plate, or 8-chamber LAB-TEKII (Invitrogen, 155409PK) for 24 hours. Cells were transfected for protein expression. Freshly prepared 4% paraformaldehyde in PBS was used for cell fixation for 20 minutes at room temperature. Cells were then wash with PBS twice and permeabilized and blocked with blocking buffer (1× PBS, 5% BSA, 0.3% Triton X-100) for 1 hour at room temperature. Cells were incubated with primary and secondary antibodies diluted with the dilution buffer (1× PBS, 1% BSA, 0.3% Triton X-100) and DAPI, sequentially, with PBS rinses three times in between. Coverslips were washed with PBS and mounted in anti-fade Diamond (Invitrogen, P36961). Images were collected on an Olympus confocal laser scanning microscope (FV3000) or Zeiss LSM 980 confocal microscopy system and exported to Fiji ImageJ for final processing and assembly. The Pearson's correlation coefficient of selected ROIs and the intensity spatial profiles were analyzed using Coloc 2 and ‘plot profile’ in Fiji ImageJ, respectively.

Statistical analysis. Statistical analyses (unpaired Student's t-tests) were performed using GraphPad Prism 8. Data were collected from at least three biological replicate experiments and presented as the mean ±s.d., ** P≤ 0.01, *** P≤ 0.001, and n.s., not significant.

Data availability. Raw data of the mass spectrometry proteomics have been deposited to the ProteomeXchange Consortium via the PRIDE (2) partner repository with the dataset identifier PXD035686.

### REFERENCES

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.
(1) Yang, X.; Qian, K. Protein O-GlcNAcylation: emerging mechanisms and functions. Nat. Rev. Mol. Cell Biol. 2017, 18 (7), 452-465.
(2) Xu, S.; Tong, M.; Suttapitugsakul, S.; Wu, R. Spatial and temporal proteomics reveals the distinct distributions and dynamics of O-GlcNAcylated proteins. Cell Rep. 2022, 39 (11), 110946.
(3) Martin, S. E. S.; Tan, Z. W.; Itkonen, H. M.; Duveau, D. Y.; Paulo, J. A.; Janetzko, J.; Boutz, P. L.; Tork, L.; Moss, F. A.; Thomas, C. J.; et al. Structure-Based Evolution of Low Nanomolar O-GlcNAc Transferase Inhibitors. J. Am. Chem. Soc. 2018, 140 (42), 13542-13545.
(4) Zhang, Z.; Tan, E. P.; VandenHull, N. J.; Peterson, K. R.; Slawson, C. O-GlcNAcase Expression is Sensitive to Changes in O-GlcNAc Homeostasis. Front Endocrinol. 2014, 5, 206.
(5) Park, S. K.; Zhou, X.; Pendleton, K. E.; Hunter, O. V.; Kohler, J. J.; O'Donnell, K. A.; Conrad, N. K. A Conserved Splicing Silencer Dynamically Regulates O-GlcNAc Transferase Intron Retention and O-GlcNAc Homeostasis. Cell Rep. 2017, 20 (5), 1088-1099.
(6) Tan, Z. W.; Fei, G.; Paulo, J. A.; Bellaousov, S.; Martin, S. E. S.; Duveau, D. Y.; Thomas, C. J.; Gygi, S. P.; Boutz, P. L.; Walker, S. O-GlcNAc regulates gene expression by controlling detained intron splicing. Nucleic Acids Res. 2020, 48 (10), 5656-5669.
(7) Yuzwa, S. A.; Vocadlo, D. J. O-GlcNAc and neurodegeneration: biochemical mechanisms and potential roles in Alzheimer's disease and beyond. Chem. Soc. Rev. 2014, 43 (19), 6839-6858.
(8) Slawson, C.; Hart, G. W. O-GlcNAc signalling: implications for cancer cell biology. Nat. Rev. Cancer 2011, 11 (9), 678-684.
(9) Ma, J.; Hart, G. W. Protein O-GlcNAcylation in diabetes and diabetic complications. Expert Rev. Proteomics 2013, 10 (4), 365-380.
(10) Ma, Z.; Vosseller, K. Cancer metabolism and elevated O-GlcNAc in oncogenic signaling. J. Biol. Chem. 2014, 289 (50), 34457-34465.
(11) Yuzwa, S. A.; Macauley, M. S.; Heinonen, J. E.; Shan, X.; Dennis, R. J.; He, Y.; Whitworth, G. E.; Stubbs, K. A.; McEachern E. J.; Davies, G. J.; et al. A potent mechanism-inspired O-GlcNAcase inhibitor that blocks phosphorylation of tau in vivo. Nat. Chem. Biol. 2008, 4 (8), 483-490.
(12) Ramirez, D. H.; Aonbangkhen, C.; Wu, H. Y.; Naftaly, J. A.; Tang, S.; O'Meara, T. R.; Woo, C. M. Engineering a Proximity-Directed O-GlcNAc Transferase for Selective Protein O-GlcNAcylation in Cells. ACS Chem. Biol. 2020, 15 (4), 1059-1066.
(13) Ge, Y.; Ramirez, D. H.; Yang, B.; D'Souza, A. K.; Aonbangkhen, C.; Wong, S.; Woo, C. M. Target protein deglycosylation in living cells by a nanobody-fused split O-GlcNAcase. Nat. Chem. Biol. 2021, 17 (5), 593-600.
(14) Gorelik, A.; Bartual, S. G.; Borodkin, V. S.; Varghese, J.; Ferenbach, A. T.; van Aalten, D. M. F. Genetic recoding to dissect the roles of site-specific protein O-GlcNAcylation. Nat. Struct. Mol. Biol. 2019, 26 (11), 1071-1077.
(15) Wang, J.; Wang, X.; Fan, X.; Chen, P. R. Unleashing the Power of Bond Cleavage Chemistry in Living Systems. ACS Cent. Sci. 2021, 7 (6), 929-943.
(16) He, J.; Fan, Z.; Tian, Y.; Yang, W.; Zhou, Y.; Zhu, Q.; Zhang, W.; Qin, W.; Yi, W. Spatiotemporal Activation of Protein O-GlcNAcylation in Living Cells. J. Am. Chem. Soc. 2022, 144 (10), 4289-4293.
(17) Kazemi, Z.; Chang, H.; Haserodt, S.; McKen, C.; Zachara, N. E. O-linked beta-N-acetylglucosamine (O-GlcNAc) regulates stress-induced heat shock protein expression in a GSK-3beta-dependent manner. J. Biol. Chem. 2010, 285 (50), 39096-39107.
(18) Levine, Z. G.; Potter, S. C.; Joiner, C. M.; Fei, G. Q.; Nabet, B.; Sonnett, M.; Zachara, N. E.; Gray, N. S.; Paulo, J. A.; Walker, S. Mammalian cell proliferation requires noncatalytic functions of O-GlcNAc transferase. Proc. Natl. Acad. Sci. U.S.A. 2021, 118 (4). DOI: 10.1073/pnas.2016778118.
(19) Schwein, P. A.; Ge, Y.; Yang, B.; D'Souza, A.; Mody, A.; Shen, D.; Woo, C. M. Writing and Erasing O-GlcNAc on Casein Kinase 2 Alpha Alters the Phosphoproteome. ACS Chem. Biol. 2022. DOI: 10.1021/acschembio.1c00987.
(20) Peck, S. H.; Chen, I.; Liu, D. R. Directed evolution of a small-molecule-triggered intein with improved splicing properties in mammalian cells. Chem. Biol. 2011, 18 (5), 619-630.
(21) Buskirk, A. R.; Ong, Y. C.; Gartner, Z. J.; Liu, D. R. Directed evolution of ligand dependence: small-molecule-activated protein splicing. Proc. Natl. Acad. Sci. U.S.A. 2004, 101 (29), 10505-10510.
(22) Topilina, N. I.; Mills, K. V. Recent advances in in vivo applications of intein-mediated protein splicing. Mob. DNA 2014, 5 (1), 5.
(23) Thompson, R. E.; Muir, T. W. Chemoenzymatic Semisynthesis of Proteins. Chem. Rev. 2020, 120 (6), 3051-3126.
(24) Davis, K. M.; Pattanayak, V.; Thompson, D. B.; Zuris, J. A.; Liu, D. R. Small molecule-triggered Cas9 protein with improved genome-editing specificity. Nat. Chem. Biol. 2015, 11 (5), 316-318.
(25) Danielian, P. S.; White, R.; Hoare, S. A.; Fawell, S. E.; Parker, M. G. Identification of residues in the estrogen receptor that confer differential sensitivity to estrogen and hydroxytamoxifen. Mol. Endocrinol. 1993, 7 (2), 232-240.
(26) Osborne, C. K. Tamoxifen in the treatment of breast cancer. N. Engl. J. Med. 1998, 339 (22), 1609-1618.
(27) Roth, C.; Chan, S.; Offen, W. A.; Hemsworth, G. R.; Willems, L. I.; King, D. T.; Varghese, V.; Britton, R.; Vocadlo, D. J.; Davies, G. J. Structural and functional insight into human O-GlcNAcase. Nat. Chem. Biol. 2017, 13 (6), 610-612.
(28) Li, B.; Li, H.; Lu, L.; Jiang, J. Structures of human O-GlcNAcase and its complexes reveal a new substrate recognition mode. Nat. Struct. Mol. Biol. 2017, 24 (4), 362-369.
(29) Elsen, N. L.; Patel, S. B.; Ford, R. E.; Hall, D. L.; Hess, F.; Kandula, H.; Kornienko, M.; Reid, J.; Selnick, H.; Shipman, J. M.; et al. Insights into activity and inhibition from the crystal structure of human O-GlcNAcase. Nat. Chem. Biol. 2017, 13 (6), 613-615.
(30) Groves, J. A.; Maduka, A. O.; O'Meally, R. N.; Cole, R. N.; Zachara, N. E. Fatty acid synthase inhibits the O-GlcNAcase during oxidative stress. J. Biol. Chem. 2017, 292 (16), 6493-6511.
(31) Taylor, R. P.; Parker, G. J.; Hazel, M. W.; Soesanto, Y.; Fuller, W.; Yazzie, M. J.; McClain, D. A. Glucose deprivation stimulates O-GlcNAc modification of proteins through up-regulation of O-linked N-acetylglucosaminyltransferase. J. Biol. Chem. 2008, 283 (10), 6050-6057.
(32) Carrillo, L. D.; Froemming, J. A.; Mahal, L. K. Targeted in vivo O-GlcNAc sensors reveal discrete compartment-specific dynamics during signal transduction. J. Biol. Chem. 2011, 286 (8), 6650-6658.
(33) Wells, L.; Gao, Y.; Mahoney, J. A.; Vosseller, K.; Chen, C.; Rosen, A.; Hart, G. W. Dynamic O-glycosylation of nuclear and cytosolic proteins: further characterization of the nucleocytoplasmic beta-N-acetylglucosaminidase, O-GlcNAcase. J. Biol. Chem. 2002, 277 (3), 1755-1761.
(34) Lubas, W. A.; Frank, D. W.; Krause, M.; Hanover, J. A. O-Linked GlcNAc transferase is a conserved nucleocytoplasmic protein containing tetratricopeptide repeats. J. Biol. Chem. 1997, 272 (14), 9316-9324.
(35)Dingwall, C.; Sharnick, S. V.; Laskey, R. A. A polypeptide domain that specifies migration of nucleoplasmin into the nucleus. Cell 1982, 30 (2), 449-458.
(36) Wen, W.; Meinkoth, J. L.; Tsien, R. Y.; Taylor, S. S. Identification of a signal for rapid export of proteins from the nucleus. Cell 1995, 82 (3), 463-473.
(37) Tan, W.; Jiang, P.; Zhang, W.; Hu, Z.; Lin, S.; Chen, L.; Li, Y.; Peng, C.; Li, Z.; Sun, A.; et al. Posttranscriptional regulation of de novo lipogenesis by glucose-induced O-GlcNAcylation. Mol. Cell 2021, 81 (9), 1890-1904 e1897.
(38) Woo, C. M.; Bertozzi, C. R. Isotope Targeted Glycoproteomics (IsoTaG) to Characterize Intact, Metabolically Labeled Glycopeptides from Complex Proteomes. Curr. Protoc. Chem. Biol. 2016, 8 (1), 59-82.
(39) Caldwell, S. A.; Jackson, S. R.; Shahriari, K. S.; Lynch, T. P.; Sethi, G.; Walker, S.; Vosseller, K.; Reginato, M. J. Nutrient sensor O-GlcNAc transferase regulates breast cancer tumorigenesis through targeting of the oncogenic transcription factor FoxM1. Oncogene 2010, 29 (19), 2831-2842.
(40) Krzeslak, A.; Forma, E.; Bernaciak, M.; Romanowicz, H.; Brys, M. Gene expression of O-GlcNAc cycling enzymes in human breast cancers. Clin. Exp. Med. 2012, 12 (1), 61-65.
(41) Ferrer, C. M.; Lynch, T. P.; Sodi, V. L.; Falcone, J. N.; Schwab, L. P.; Peacock, D. L.; Vocadlo, D. J.; Seagroves, T. N.; Reginato, M. J. O-GlcNAcylation regulates cancer metabolism and survival stress signaling via regulation of the HIF-1 pathway. Mol. Cell 2014, 54 (5), 820-831.
(42) Mills, J. N.; Rutkovsky, A. C.; Giordano, A. Mechanisms of resistance in estrogen receptor positive breast cancer: overcoming resistance to tamoxifen/aromatase inhibitors. Curr. Opin. Pharmacol. 2018, 41, 59-65.
(43) Kanwal, S.; Fardini, Y.; Pagesy, P.; N′Tumba-Byn, T.; Pierre-Eugene, C.; Masson, E.; Hampe, C.; Issad, T. O-GlcNAcylation-inducing treatments inhibit estrogen receptor alpha expression and confer resistance to 4—OH-tamoxifen in human breast cancer-derived MCF-7 cells. PloS One 2013, 8 (7), e69150. DOI: 10.1371/journal.pone.0069150.
(44) Ramirez, D. H.; Ge, Y.; Woo, C. M. O-GlcNAc Engineering on a Target Protein in Cells with Nanobody-OGT and Nanobody-splitOGA. Curr. Protoc. 2021, 1 (5), e117. DOI: 10.1002/cpz1.117.
(45) Zorn, J. A.; Wells, J. A. Turning enzymes ON with small molecules. Nat. Chem. Biol. 2010, 6 (3), 179-188.
(46) Dagliyan, O.; Shirvanyants, D.; Karginov, A. V.; Ding, F.; Fee, L.; Chandrasekaran, S. N.; Freisinger, C. M.; Smolen, G. A.; Huttenlocher, A.; Hahn, K. M.; et al. Rational design of a ligand-controlled protein conformational switch. Proc. Natl. Acad. Sci. U.S.A. 2013, 110 (17), 6800-6804.
(47) Feil, S.; Valtcheva, N.; Feil, R. Inducible Cre mice. Methods Mol. Biol. 2009, 530, 343-363.
(48) El Mjiyad, N.; Caro-Maldonado, A.; Ramirez-Peinado, S.; Munoz-Pinedo, C. Sugar-free approaches to cancer cell killing. Oncogene 2011, 30 (3), 253-264.
(49) Yu, F.; Zhang, Q.; Liu, H.; Liu, J.; Yang, S.; Luo, X.; Liu, W.; Zheng, H.; Liu, Q.; Cui, Y.; et al. Dynamic O-GlcNAcylation coordinates ferritinophagy and mitophagy to activate ferroptosis. Cell Discov. 2022, 8 (1), 40.
(50) Paunovska, K.; Loughrey, D.; Dahlman, J. E. Drug delivery systems for RNA therapeutics. Nat. Rev. Genet. 2022, 23 (5), 265-280.
(51) Thompson, J. W.; Griffin, M. E.; Hsich-Wilson, L. C. Methods for the Detection, Study, and Dynamic Profiling of O-GlcNAc Glycosylation. Methods Enzymol. 2018, 598, 101-135. DOI: 10.1016/bs.mic.2017.06.009.
(52) Perez-Riverol, Y.; Csordas, A.; Bai, J.; Bernal-Llinares, M.; Hewapathirana, S.; Kundu, D. J.; Inuganti, A.; Griss, J.; Mayer, G.; Eisenacher, M.; et al. The PRIDE database and related tools and resources in 2019: improving support for quantification data. Nucleic Acids Res. 2019, 47 (D1), D442-D450. DOI: 10.1093/nar/gky 1106.
(53) Gyorffy, B. Survival analysis across the entire transcriptome identifies biomarkers with the highest prognostic power in breast cancer. Comput. Struct. Biotechnol. J. 2021, 19, 4101-4109. DOI: 10.1016/j.csbj.2021.07.014.

### Equivalents and Scope

Those skilled in the art will recognize or be able to ascertain using no more than routine experimentation many equivalents to the specific embodiments of the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity, those embodiments have not been specifically set forth in haec verba herein. Thus, for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A glycosyl hydrolase comprising an intein.

2. The glycosyl hydrolase of claim 1, wherein the activity of the glycosyl hydrolase is disrupted by the intein.

3. The glycosyl hydrolase of claim 1, wherein the activity of the glycosyl hydrolase is restored upon excision of the intein.

4. The glycosyl hydrolase of claim 1, wherein the glycosyl hydrolase is an O-GlcNAcase (OGA).

5-9. (canceled)

10. The glycosyl hydrolase of claim 1, wherein the glycosyl hydrolase comprises the amino acid sequence of SEQ ID NO: 107, or an amino acid sequence at least 80%, at least 85%, at least 90%, at least 95%, or at least 99% identical to the amino acid sequence of SEQ ID NO: 107.

11. The glycosyl hydrolase of claim 1, wherein the intein is a ligand-dependent intein.

12. The glycosyl hydrolase of claim 11, wherein the ligand-dependent intein is excised from the glycosyl hydrolase upon binding of a ligand.

13-17. (canceled)

18. The glycosyl hydrolase of claim 1, wherein the intein is inserted at or replaces a cysteine within the glycosyl hydrolase.

19-21. (canceled)

22. The glycosyl hydrolase of claim 1, wherein the glycosyl hydrolase is fused to a nuclear localization sequence (NLS).

23. (canceled)

24. The glycosyl hydrolase of claim 1, wherein the glycosyl hydrolase is fused to a nuclear export sequence (NES).

25. (canceled)

26. The glycosyl hydrolase of claim 1, wherein the glycosyl hydrolase is fused to a targeting molecule.

27-32. (canceled)

33. A pharmaceutical composition comprising the glycosyl hydrolase of claim 1 and a pharmaceutically acceptable excipient.

34. A polynucleotide encoding the glycosyl hydrolase of claim 1.

35. A vector comprising the polynucleotide of claim 34.

36. A cell comprising the glycosyl hydrolase of claim 1.

37. (canceled)

38. A method of deglycosylating a target protein, the method comprising:
(i) contacting a target protein containing a sugar moiety with a glycosyl hydrolase of claim 1; and
(ii) contacting the glycosyl hydrolase with a ligand, thereby excising the intein from the glycosyl hydrolase and restoring its activity;
wherein the sugar moiety is removed from the target protein upon restoration of the activity of the glycosyl hydrolase.

39-43. (canceled)

44. A method of studying the effects of glycosylation on protein function in a cell, the method comprising:
(i) administering to the cell the glycosyl hydrolase of claim 1; and
(ii) contacting the glycosyl hydrolase with a ligand, thereby excising the intein from the glycosyl hydrolase and restoring its activity.

45. A method of treating a glycosylation-associated disease in a subject, the method comprising:
(i) administering to the subject a therapeutically effective amount of a glycosyl hydrolase of claim 1; and
(ii) contacting the glycosyl hydrolase with a ligand, thereby excising the intein from the glycosyl hydrolase and restoring its activity.

46. A method of reducing drug resistance in a cell, the method comprising:
(i) administering to the cell the glycosyl hydrolase of claim 1; and
(ii) contacting the glycosyl hydrolase with a ligand, thereby excising the intein from the glycosyl hydrolase, wherein glycosyl hydrolase modulates the glycosylation state of one or more proteins in the cell thereby reducing the drug resistance.

47. A method of sensitizing a cell to a desirable therapeutic outcome, the method comprising:
(i) administering to the cell the glycosyl hydrolase of claim 1; and
(ii) contacting the glycosyl hydrolase with a ligand, thereby excising the intein from the glycosyl hydrolase, wherein glycosyl hydrolase modulates the glycosylation state of one or more proteins in the cell thereby sensitizing a cell to a desirable therapeutic outcome.

48. (canceled)


SEQUENCE LISTING (122 sequences)

SEQ ID NO: 1 - protein, 15 aa, synthetic construct
GSGSGSGSGSGSGSG

SEQ ID NO: 2 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 3 - protein, 413 aa, synthetic construct
CLAEGTRIFDPVTGTTHRIEDVVDGRKPIHVVAVAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYTNVVPLYDLLLEMLDAHRLHAGGSGASRVQAFADALDDKFLHDMLAEELRYSVIREVLPTRRARTFDLEVEELHTLVAEGVVVHNC

SEQ ID NO: 4 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 5 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 6 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 7 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYNPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 8 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 9 - protein, 413 aa, synthetic construct
CLAEGTRIFDPVTGTTHRIEDVVDGRKPIHVVAVAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 10 - protein, 16 aa, synthetic construct
MAPKKKRKVGIHRGVP

SEQ ID NO: 11 - protein, 7 aa, synthetic construct
PKKKRKV

SEQ ID NO: 12 - protein, 19 aa, synthetic construct
MKRTADGSEFESPKKKRKV

SEQ ID NO: 13 - protein, 30 aa, synthetic construct
MDSLLMNRRKFLYQFKNVRWAKGRRETYLC

SEQ ID NO: 14 - protein, 20 aa, synthetic construct
AVKRPAATKKAGQAKKKKLD

SEQ ID NO: 15 - protein, 25 aa, synthetic construct
MSRRRKANPTKLSENAKKLAKEVEN

SEQ ID NO: 16 - protein, 9 aa, synthetic construct
PAAKRVKLD

SEQ ID NO: 17 - protein, 9 aa, synthetic construct
KLKIKRPVK

SEQ ID NO: 18 - protein, 8 aa, synthetic construct
VSRKRPRP

SEQ ID NO: 19 - protein, 10 aa, synthetic construct
EGAPPAKRAR

SEQ ID NO: 20 - protein, 12 aa, synthetic construct
PPQPKKKPLDGE

SEQ ID NO: 21 - protein, 21 aa, synthetic construct
SGGSKRTADGSEFEPKKKRKV

SEQ ID NO: 22 - protein, 18 aa, synthetic construct
KRTADGSEFESPKKKRKV

SEQ ID NO: 23 - protein, 14 aa, synthetic construct
MEELSQALASSFSV

SEQ ID NO: 24 - protein, 12 aa, synthetic construct
PLQLPPLERLTL

SEQ ID NO: 25 - protein, 12 aa, synthetic construct
NELALKLAGLDI

SEQ ID NO: 26 - protein, 15 aa, synthetic construct
ERFEMFRELNEALEL

SEQ ID NO: 27 - protein, 15 aa, synthetic construct
DHAEKVAEKLEALSV

SEQ ID NO: 28 - protein, 15 aa, synthetic construct
QLVEELLKIICAFQL

SEQ ID NO: 29 - protein, 15 aa, synthetic construct
TNLEALQKKLEELEL

SEQ ID NO: 30 - protein, 15 aa, synthetic construct
DVKEEMTSALATMRV

SEQ ID NO: 31 - protein, 15 aa, synthetic construct
STNGSLAAEFRHLQL

SEQ ID NO: 32 - protein, 15 aa, synthetic construct
PSVQELTEQIHRLLM

SEQ ID NO: 33 - protein, 15 aa, synthetic construct
MNFKELKDFLKELNI

SEQ ID NO: 34 - protein, 15 aa, synthetic construct
ENFEILMKLKESLEL

SEQ ID NO: 35 - protein, 14 aa, synthetic construct
FETVYELTKMCTIR

SEQ ID NO: 36 - protein, 15 aa, synthetic construct
SGKASSSLGLQDFDL

SEQ ID NO: 37 - protein, 15 aa, synthetic construct
PKYSDIDVDGLCSEL

SEQ ID NO: 38 - protein, 15 aa, synthetic construct
VDLACTPTDVRDVDI

SEQ ID NO: 39 - protein, 15 aa, synthetic construct
YGEKTTQRDLTELEI

SEQ ID NO: 40 - protein, 15 aa, synthetic construct
RRIYDITNVLEGIGL

SEQ ID NO: 41 - protein, 15 aa, synthetic construct
AKIIPYSGLLLVITV

SEQ ID NO: 42 - protein, 15 aa, synthetic construct
LRSEEVHWLHVDMGV

SEQ ID NO: 43 - protein, 15 aa, synthetic construct
LQSEEVHWLHLDMGV

SEQ ID NO: 44 - protein, 15 aa, synthetic construct
LQVRKYSLDLASLIL

SEQ ID NO: 45 - protein, 15 aa, synthetic construct
AGVEAIIRILQQLLF

SEQ ID NO: 46 - protein, 15 aa, synthetic construct
TGVEALIRILQQLLF

SEQ ID NO: 47 - protein, 15 aa, synthetic construct
IVLNQLCVRFFGLDL

SEQ ID NO: 48 - protein, 15 aa, synthetic construct
SLGGFEITPPVVLRL

SEQ ID NO: 49 - protein, 15 aa, synthetic construct
EAIQDLCLAVEEVSL

SEQ ID NO: 50 - protein, 15 aa, synthetic construct
DELLQVLRMMVGVNI

SEQ ID NO: 51 - protein, 15 aa, synthetic construct
SVMLAVQEGIDLLTF

SEQ ID NO: 52 - protein, 10 aa, synthetic construct
LSSHFQELSI

SEQ ID NO: 53 - protein, 15 aa, synthetic construct
QSTHVDIRTLEDLLM

SEQ ID NO: 54 - protein, 15 aa, synthetic construct
ESSAEDLRTLQQLFL

SEQ ID NO: 55 - protein, 15 aa, synthetic construct
EFSLPTHHTVRLIRV

SEQ ID NO: 56 - protein, 15 aa, synthetic construct
MSSGYYLGEILRLAL

SEQ ID NO: 57 - protein, 15 aa, synthetic construct
DTVLDILRDFFELRL

SEQ ID NO: 58 - protein, 15 aa, synthetic construct
NSVNEILSEFYYVRL

SEQ ID NO: 59 - protein, 15 aa, synthetic construct
CAFLSVKKQFEELTL

SEQ ID NO: 60 - protein, 15 aa, synthetic construct
ISPEHVIQALESLGF

SEQ ID NO: 61 - protein, 15 aa, synthetic construct
AHWMRQLVSFQKLKL

SEQ ID NO: 62 - protein, 15 aa, synthetic construct
ATRELDELMASLSDF

SEQ ID NO: 63 - protein, 15 aa, synthetic construct
YQNIELITFINALKL

SEQ ID NO: 64 - protein, 15 aa, synthetic construct
FNATAVVRHMRKLQL

SEQ ID NO: 65 - protein, 15 aa, synthetic construct
SGIFGLVTNLEELEV

SEQ ID NO: 66 - protein, 15 aa, synthetic construct
EESYTLNSDLARLGV

SEQ ID NO: 67 - protein, 15 aa, synthetic construct
EESYDLTSHLARLGV

SEQ ID NO: 68 - protein, 15 aa, synthetic construct
GIQQAHAEQLANMRI

SEQ ID NO: 69 - protein, 15 aa, synthetic construct
AAEPVILDLRDLFQL

SEQ ID NO: 70 - protein, 10 aa, synthetic construct
MEGCVSNLMV

SEQ ID NO: 71 - protein, 9 aa, synthetic construct
EGCVSNLMV

SEQ ID NO: 72 - protein, 15 aa, synthetic construct
DMDFLRNLFSQTLSL

SEQ ID NO: 73 - protein, 15 aa, synthetic construct
EQLLEIVHDLENLSL

SEQ ID NO: 74 - protein, 15 aa, synthetic construct
NVMKYFTDLFDYLPL

SEQ ID NO: 75 - protein, 15 aa, synthetic construct
KVYPIILRLGSNLSL

SEQ ID NO: 76 - protein, 15 aa, synthetic construct
YAGFSLPHAILRIDL

SEQ ID NO: 77 - protein, 15 aa, synthetic construct
EIVRDIKEKLCYVAL

SEQ ID NO: 78 - protein, 15 aa, synthetic construct
EAINKLESNLRELQI

SEQ ID NO: 79 - protein, 15 aa, synthetic construct
EAINKLENNLRELQI

SEQ ID NO: 80 - protein, 15 aa, synthetic construct
SDQKQEQLLLKKMYL

SEQ ID NO: 81 - protein, 15 aa, synthetic construct
KQVLWDRTFSLFQQL

SEQ ID NO: 82 - protein, 15 aa, synthetic construct
AQLQNLTKRIDSLPL

SEQ ID NO: 83 - protein, 15 aa, synthetic construct
NDENEHQLSLRTVSL

SEQ ID NO: 84 - protein, 15 aa, synthetic construct
ISFTEFVKVLEKVDV

SEQ ID NO: 85 - protein, 14 aa, synthetic construct
MESAITLWQFLLQL

SEQ ID NO: 86 - protein, 15 aa, synthetic construct
VPKELMQQIENFEKI

SEQ ID NO: 87 - protein, 15 aa, synthetic construct
QARFILEKIDGKIII

SEQ ID NO: 88 - protein, 15 aa, synthetic construct
QVKFIKMIIEKELTV

SEQ ID NO: 89 - protein, 15 aa, synthetic construct
NHRMKNLREISQLGI

SEQ ID NO: 90 - protein, 15 aa, synthetic construct
NHRVKKLNEISKLGI

SEQ ID NO: 91 - protein, 15 aa, synthetic construct
TEKHLQKYLRQDLRL

SEQ ID NO: 92 - protein, 15 aa, synthetic construct
RQERKRPLLDLHIEL

SEQ ID NO: 93 - protein, 15 aa, synthetic construct
ANMRIQDLKVSLKPL

SEQ ID NO: 94 - protein, 15 aa, synthetic construct
ATMRVDYEQIKIKKI

SEQ ID NO: 95 - protein, 15 aa, synthetic construct
LQGEEFVCLKSIILL

SEQ ID NO: 96 - protein, 15 aa, synthetic construct
THYGQKAILFLPLPV

SEQ ID NO: 97 - protein, 15 aa, synthetic construct
PSAHEITGLADSLQL

SEQ ID NO: 98 - protein, 15 aa, synthetic construct
VRLHDVLHSDKKLTL

SEQ ID NO: 99 - protein, 15 aa, synthetic construct
LINRNGELKLANFGL

SEQ ID NO: 100 - protein, 15 aa, synthetic construct
LEPLKKLECLKSLDL

SEQ ID NO: 101 - DNA, 22 bp, synthetic construct
  source 1..22: mol_type = other DNA
caggaaggctattgctgagagg

SEQ ID NO: 102 - DNA, 23 bp, synthetic construct
  source 1..23: mol_type = other DNA
cggaactcacatatcctacacgc

SEQ ID NO: 103 - DNA, 23 bp, synthetic construct
  source 1..23: mol_type = other DNA
gcaagagtttggtgtgcctcatc

SEQ ID NO: 104 - DNA, 22 bp, synthetic construct
  source 1..22: mol_type = other DNA
gtgctgcaactaaaggagtccc

SEQ ID NO: 105 - DNA, 22 bp, synthetic construct
  source 1..22: mol_type = other DNA
gtctcctctgacttcaacagcg

SEQ ID NO: 106 - DNA, 22 bp, synthetic construct
  source 1..22: mol_type = other DNA
accaccctgttgctgtagccaa

SEQ ID NO: 107 - protein, 592 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRGSGSGSGSGSGSGSGEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 108 - protein, 1004 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRGSGSGSGSGSGSGSGEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 109 - protein, 1004 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFNDIDHNMCLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRGSGSGSGSGSGSGSGEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 110 - protein, 1020 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRGSGSGSGSGSGSGSGEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPKRPAATKKAGQAKKKK

SEQ ID NO: 111 - protein, 1015 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRGSGSGSGSGSGSGSGEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPLALKLAGLDIN

SEQ ID NO: 112 - protein, 939 aa, synthetic construct
MASMQKLISEEDLLMAMEARIRSTVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRQVAHSGAKASVVDGTPLVAAPSLNATTVVTTVYQEPIMSQGAALSGEPTTLTKEEEKKQPDEEPMDMVVEKQEETDHKNDNQILSEIVEAKMAEELKPMDTDKESIAESKSPEMSMQEDCISDIAPMQTDEQTNKEQFVPGPNEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPPLTPTSKVYTIRPYFPKDEASVYKICREMYDDGVGLPFQSQPDLIGDKLVGGLLSLSLDYCFVLEDEDGICGYALGTVDVTPFIKKCKISWIPFMQEKYTKPNGDKELSEAEKIMLSFHEEQEVLPETFLANFPSLIKMDIHKKVTDPSVAKSMMACLLSSLKANGSRGAFCEVRPDDKRILEFYSKLGCFEIAKMEGFPKDVVILGRSL

SEQ ID NO: 113 - DNA, 2820 bp, synthetic construct
  source 1..2820: mol_type = other DNA
atggcatcaatgcagaagctgatctcagaggaggacctgcttatggccatggaggcccgaattcggtcgaccgtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggcaagttgcacacagtggagctaaagcaagtgtagttgatgggactcctttagttgcagcaccctctttaaatgccacaaccgtagtaacaacagtttatcaggagcccattatgagccagggagcagccttgagtggtgagcctactactctgaccaaggaagaagaaaagaaacagcctgatgaagaacccatggacatggtggtggaaaaacaagaagaaacggaccacaagaatgacaatcaaatactgagtgaaattgttgaagcgaaaatggcagaggaattgaaaccaatggacactgataaagagagcatagctgaatcaaaatccccagagatgtccatgcaagaagattgtattagtgacattgcccccatgcaaactgatgaacagacaaacaaggagcagtttgtgccaggtccaaatgaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccacctccactgactcctacctccaaagtttatactatcagaccttattttcctaaggatgaggcatccgtgtacaagatttgcagagaaatgtatgacgatggagtgggtttaccctttcaaagtcagcctgatcttattggagacaagttagtaggagggctgctttccctcagcctggattactgctttgtcctagaagatgaagatggcatatgtggttatgccttgggcactgtagatgtgaccccctttattaaaaaatgtaaaatttcctggatccccttcatgcaggagaagtataccaagccaaatggtgacaaggaactctctgaggctgagaaaataatgttgagtttccatgaagaacaggaagtactgccagaaactttccttgctaatttcccttctctgataaagatggacattcacaaaaaagtaactgacccaagtgtggccaaaagcatgatggcttgcctcctgtcttcactgaaggctaatggctcccggggagctttctgtgaagtgagaccagatgataaaagaattctggaattttacagcaagttaggatgttttgaaattgcaaaaatggaaggatttccaaaggatgtggttatacttggtcggagcctgtaa

SEQ ID NO: 114 - DNA, 1245 bp, synthetic construct
  source 1..1245: mol_type = other DNA
atggagcagaagctgatcagcgaggaggacctggcgatcgcaatggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggtaa

SEQ ID NO: 115 - protein, 414 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSR

SEQ ID NO: 116 - DNA, 1245 bp, synthetic construct
  source 1..1245: mol_type = other DNA
atggagcagaagctgatcagcgaggaggacctggcgatcgcaatggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttaatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggtaa

SEQ ID NO: 117 - protein, 414 aa, synthetic construct
MEQKLISEEDLAIAMVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFNDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSR

SEQ ID NO: 118 - DNA, 534 bp, synthetic construct
  source 1..534: mol_type = other DNA
atggcatacccatacgatgttccagattacgctgcgatcgcagaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccaccttaa

SEQ ID NO: 119 - protein, 177 aa, synthetic construct
MAYPYDVPDYAAIAEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 120 - DNA, 957 bp, synthetic construct
  source 1..957: mol_type = other DNA
atggcatacccatacgatgttccagattacgctgcgatcgcacaggtgcagctggtggagtctggaggagctctggtgcagcctggaggaagcctgcgcctgagctgtgcagctagcggatttcctgtgaaccgctacagcatgcgctggtaccgccaggctcctggtaaagagcgcgagtgggtggctggaatgagcagcgctggagatcgcagcagctacgaggacagcgtgaaaggacgctttacaatcagccgcgatgatgctcgcaacacagtgtacctgcagatgaactctctgaaacctgaggacactgctgtgtactactgtaacgtgaacgtgggtttcgagtactggggacagggaacacaggtgacagtgagctctggcgcgccagaggcagctgcaaaggaggcagctgcaaaggaggcagctgcaaaggaggcagctgcaaagttaattaaggaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctaaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccaccttaa

SEQ ID NO: 121 - protein, 318 aa, synthetic construct
MAYPYDVPDYAAIAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPEAAAKEAAAKEAAAKEAAAKLIKEKPLYTAEPVTLEDLQLLADLFYLPYEHGPKGAQMLREFQWLRANSSVVSVNCKGKDSKKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPP

SEQ ID NO: 122 - protein, 5 aa, synthetic construct
EAAAK